(19) United States
(12) Patent Application Publication
(10) Pub. No.: US 2024/0174992 A1
(43) Pub. Date: May 30, 2024

(54) SPLIT MODIFIED DEHALOGENASE VARIANTS

(71) Applicant: Promega Corporation, Madison, WI (US)

(72) Inventors: Michael P. Killoran, Madison, WI (US); Evan Glasgow, Madison, WI (US); Lance P. Encell, Madison, WI (US); Thomas Kirkland, Madison, WI (US); Rahele Esmatpour, Madison, WI (US)

(21) Appl. No.: 18/312,117

(22) Filed: May 4, 2023

Related U.S. Application Data
(60) Provisional application No. 63/338,323, filed on May 4, 2022.

(51) Int. Cl. C12N 9/14; G01N 33/68

(52) U.S. Cl. CPC C12N 9/14; C12Y 308/01005; G01N 33/6845

(57) ABSTRACT

Provided herein are peptide and polypeptide sequences that structurally assemble to form active, modified dehalogenase structures capable of binding to a haloalkyl ligand. In particular, provided herein are split dehalogenase variants that assemble through structural complementation into active dehalogenase complexes, and systems and methods of use thereof.

### CROSS-REFERENCE TO RELATED APPLICATIONS

This application claims the benefit of U.S. Provisional Patent Application No. 63/338,323, filed on May 4, 2022, which is incorporated by reference herein.

### SEQUENCE LISTING

The instant application contains a Sequence Listing which has been submitted electronically in xml. format and is hereby incorporated by reference in its entirety. Said xml. copy created on Jan. 25, 2024, is named 39886_202_SequenceListing.xml and the file size is 3,224,550 bytes.

### MEGA TABLE

The specification includes two lengthy tables. Table 1 and Table 2 have been submitted via EFS-Web in electronic format as follows: File name: TABLE_1_SmHTs.txt, Date created: May 4, 2023, 2023, File size: 64,623Bytes; and File name: TABLE_2_LgHTs.txt, Date created: May 3, 2023, File size: 2,108,573 Bytes. The content of Table 1 and Table 2 is hereby incorporated by reference in its entirety.

### FIELD

Provided herein are peptide and polypeptide sequences that structurally assemble to form active, modified dehalogenase structures capable of binding to a haloalkyl ligand. In particular, provided herein are split dehalogenase variants that assemble through structural complementation into active dehalogenase complexes, and systems and methods of use thereof.

### BACKGROUND

The utility of self-labeling protein systems, such as HALOTAG and its chloroalkane-based ligands, has continually expanded during their lifetime as research tools. Genetic fusions to HALOTAG as a general strategy have enabled a broad range of applications including fluorescence labeling for cell biology and imaging, recombinant protein purification, biosensors and diagnostics, energy transfer technologies (BRET, FRET), and targeted protein degradation for therapeutics (PROTACs). The development of new fluorophores and fluorogenic dyes (such as the JANELIA FLUOR dyes) as chloroalkane conjugates serves as one example highlighting renewed interest in HALOTAG for fluorescence detection in cell imaging applications. The advantages of such dyes in brightness, photostability, sensitivity, and far-red spectral detection over conventional tools such as widely-used fluorescent proteins is particularly apparent in challenging or highly sensitive imaging applications in endogenous biology. As chloroalkane conjugates, they can take advantage of the self-labeling activity of HALOTAG to measure protein abundance and localization in a target-specific manner through genetic fusion. However, there is a lack of available tools capable of measuring important functional dynamics with cell imaging as well, such as protein interactions or changes in metabolite concentration, which can take advantage of these improvements in fluorescence detection. What is needed in the field are tools for controlling self-labeling activity in a dynamic way, in systems such as HALOTAG.

### SUMMARY

Provided herein are peptide and polypeptide sequences that structurally assemble to form active, modified dehalogenase structures capable of binding to a haloalkyl ligand. In particular, provided herein are split dehalogenase variants that assemble through structural complementation into active dehalogenase complexes, and systems and methods of use thereof.

In some embodiments, provided herein are compositions comprising split variants of a polypeptide comprising at least 70% sequence similarity (e.g., at least 70%, at least 75%, at least 80%, at least 85%, at least 90%, at least 95%) with SEQ ID NO: 1. In some embodiments, the split variant comprises at least 70% (e.g., at least 70%, at least 75%, at least 80%, at least 85%, at least 90%, at least 95%, 100%) sequence identity with SEQ ID NO: 1.

In some embodiments, a split variant is a binary system comprising first and second fragments. In some embodiments, the split variant comprises: (i) a first fragment of a polypeptide comprising at least 70% sequence similarity (e.g., at least 70%, at least 75%, at least 80%, at least 85%, at least 90%, at least 95%) with a first portion of SEQ ID NO: 1, and (ii) a second fragment of a polypeptide comprising at least 70% sequence similarity (e.g., at least 70%, at least 75%, at least 80%, at least 85%, at least 90%, at least 95%, 100%) with a second portion of SEQ ID NO: 1. In some embodiments, the first fragment comprises at least 70% (e.g., at least 70%, at least 75%, at least 80%, at least 85%, at least 90%, at least 95%, 100%) sequence identity with the first portion of SEQ ID NO: 1. In some embodiments, the second fragment comprises at least 70% (e.g., at least 70%, at least 75%, at least 80%, at least 85%, at least 90%, at least 95%, 100%) sequence identity with the second portion of SEQ ID NO: 1. In some embodiments, the first fragment and the second fragment collectively comprise amino acid sequence corresponding to at least 80% of the length of SEQ ID NO: 1 (e.g., at least 80%, at least 85%, at least 90%, at least 95%, 100%).

In some embodiments, the first and second fragments each comprise at least 70% (e.g., at least 70%, at least 75%, at least 80%, at least 85%, at least 90%, at least 95%, 100%) sequence identity with one of SEQ ID NOS: 2-577. In some embodiments, the first and second fragments each comprise at 100% sequence similarity with one of SEQ ID NOS: 2-577. In some embodiments, the first and second fragments each comprise at 100% sequence identity with one of SEQ ID NOS: 2-577.

In some embodiments, the first fragment comprises at least 70% (e.g., at least 70%, at least 75%, at least 80%, at least 85%, at least 90%, at least 95%, 100%) sequence similarity with one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, and 576; and the second fragment comprises at least 70% (e.g., at least 70%, at least 75%, at least 80%, at least 85%, at least 90%, at least 95%, 100%) sequence similarity with one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, and 577.

In some embodiments, the first fragment comprises at least 70% (e.g., at least 70%, at least 75%, at least 80%, at least 85%, at least 90%, at least 95%, 100%) sequence identity with the first reference sequence selected from one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, and 576.

In some embodiments, the second fragment comprises at least 70% (e.g., at least 70%, at least 75%, at least 80%, at least 85%, at least 90%, at least 95%, 100%) sequence identity with the second reference sequence selected from one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, and 577.

In some embodiments, the first and second fragments exhibit enhancement of one or more traits compared to the first and second reference sequences, wherein the traits are selected from: affinity for each other, expression, intracellular solubility, intracellular stability, and activity when combined.

In some embodiments, the split variant comprises a split (“sp”) site at a position corresponding to any position between positions 5 and 290 (e.g., positions 19-34). In some embodiments, the split variant comprises a sp site at a position corresponding to a position between positions 5 and 13 (e.g., 5, 6, 7, 8, 9, 10, 11, 12, 13, or ranges therebetween), 36 and 51 (e.g., 36, 37, 38, 39, 40, 41, 42, 43, 44, 45, 46, 47, 48, 49, 50, 51, or ranges therebetween), 63 and 72 (e.g., 63, 64, 65, 66, 67, 68, 69, 70, 71, 72, or ranges therebetween), 84 and 92 (e.g., 84, 85, 86, 87, 88, 89, 90, 91, 92, or ranges therebetween), 104 and 130 (e.g., 104, 105, 106, 107, 108, 109, 110, 111, 112, 113, 114, 115, 116, 117, 118, 119, 120, 121, 122, 123, 124, 125, 126, 127, 128, 129, 130, or ranges therebetween), 142 and 148 (e.g., 142, 143, 144, 145, 146, 147, 148, and ranges therebetween), 160 and 174 (e.g., 160, 161, 162, 163, 164, 165, 166, 167, 168, 169, 170, 171, 172, 173, 174, or ranges therebetween), 186 and 189 (e.g., 186, 187, 188, 189, or ranges therebetween), 201 and 203 (e.g., 201, 202, 203, or ranges therebetween), 221 and 229 (e.g., 221, 222, 223, 224, 225, 226, 227, 228, 229, or ranges therebetween), or 269 and 290 (e.g., 269, 270, 271, 272, 273, 274, 275, 276, 277, 278, 279, 280, 281, 282, 283, 284, 285, 286, 287, 288, 289, or 290 or ranges therebetween) of SEQ ID NO: 1.

In some embodiments, the split variant is capable of forming a covalent bond with a haloalkane substrate.

In some embodiments, the split variant comprises 100% sequence identity to SEQ ID NO: 1.

In some embodiments, the split variant comprises deletions of up to 40 amino acids (e.g., 1, 2, 3, 4, 5, 6, 7, 8, 9, 10, 11, 12, 13, 14, 15, 16, 17, 18, 19, 20, 25, 30, 35, 40, or ranges therebetween) at positions corresponding to one or more of the N-terminus of SEQ ID NO: 1, the C-terminus of SEQ ID NO: 1, and either side of the sp site. In some embodiments, the split variant comprises duplicated sequences of up to 40 amino acids (e.g., 1, 2, 3, 4, 5, 6, 7, 8, 9, 10, 11, 12, 13, 14, 15, 16, 17, 18, 19, 20, 25, 30, 35, 40, or ranges therebetween) at positions corresponding to either side of the sp site.

In some embodiments, provided herein are compositions comprising (i) a peptide having at least 70% (e.g., 70%, 75%, 80%, 85%, 90%, 95%, 100%) sequence similarity with one or more of SEQ ID NOS: 578-1187, and (ii) a polypeptide having at least 70% sequence similarity with one or more of SEQ ID NOS: 1188-3033; wherein a complex of the peptide and polypeptide is capable of forming a covalent bond with a haloalkane substrate. In some embodiments, the peptide has at least 70% (e.g., 70%, 75%, 80%, 85%, 90%, 95%, 100%) sequence identity with one of SEQ ID NOS: 578-1187. In some embodiments, the peptide has at least 70% (e.g., 70%, 75%, 80%, 85%, 90%, 95%, 100%) sequence identity with one of SEQ ID NOS: 1188-3033.

In some embodiments, provided herein are peptides having at least 70% (e.g., 70%, 75%, 80%, 85%, 90%, 95%, 100%) sequence similarity with one or more of SEQ ID NOS: 578-1187. In some embodiments, provided herein are peptides having at least 70% (e.g., 70%, 75%, 80%, 85%, 90%, 95%, 100%) sequence identity with one of SEQ ID NOS: 578-1187. In some embodiments, the peptides are capable of forming a complex (e.g., facilitated or unfacilitated) with a polypeptide of SEQ ID NO: 1188, wherein the complex is capable of forming a covalent bond with a haloalkane substrate.

In some embodiments, provided herein are peptides or polypeptides comprising at least 70% (e.g., at least 70%, at least 75%, at least 80%, at least 85%, at least 90%, at least 95%, 100%) sequence similarity with one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, and 576; wherein the peptide or polypeptide is capable of interacting with a peptide or polypeptide selected from one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, and 577 to form a modified dehalogenase complex, and wherein the is capable of forming a covalent bond with a haloalkane substrate. In some embodiments, the peptide or polypeptide comprises at least 70% (e.g., at least 70%, at least 75%, at least 80%, at least 85%, at least 90%, at least 95%, 100%) sequence identity with one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, and 576.

In some embodiments, provided herein are peptides or polypeptides comprising at least 70% (e.g., at least 70%, at least 75%, at least 80%, at least 85%, at least 90%, at least 95%, 100%) sequence similarity with one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, and 577; wherein the peptide or polypeptide is capable of interacting with a peptide or polypeptide selected from one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, and 576 to form a modified dehalogenase complex, and wherein the modified dehalogenase complex is capable of forming a covalent bond with a haloalkane substrate. In some embodiments, the peptide or polypeptide comprises at least 70% (e.g., at least 70%, at least 75%, at least 80%, at least 85%, at least 90%, at least 95%, 100%) sequence identity with one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, and 577.

In some embodiments, provided herein are peptides comprising at least 70% (e.g., at least 70%, at least 75%, at least 80%, at least 85%, at least 90%, at least 95%, 100%) sequence similarity to one of SEQ ID NOS: 578-1187; wherein the peptide is capable of interacting with a polypeptide selected from one of SEQ ID NOS: 1188-3033 to form a modified dehalogenase complex, and wherein the modified dehalogenase complex is capable of forming a covalent bond with a haloalkane substrate. In some embodiments, the peptides comprise at least 70% (e.g., at least 70%, at least 75%, at least 80%, at least 85%, at least 90%, at least 95%, 100%) sequence identity to one of SEQ ID NOS: 578-1187.

In some embodiments, provided herein are peptides comprising 100% sequence identity with SEQ ID NO: 3034 or 3035.

In some embodiments, provided herein are polypeptides comprising at least 70% (e.g., at least 70%, at least 75%, at least 80%, at least 85%, at least 90%, at least 95%, 100%) sequence similarity to one of SEQ ID NOS: 1188-3033; wherein the polypeptide is capable of interacting with a peptide selected from one of SEQ ID NOS: 578-1187, 3034, or 3035) to form a modified dehalogenase complex, and wherein the modified dehalogenase complex is capable of forming a covalent bond with a haloalkane substrate. In some embodiments, the polypeptide comprises at least 70% (e.g., at least 70%, at least 75%, at least 80%, at least 85%, at least 90%, at least 95%, 100%) sequence identity to one of SEQ ID NOS: 1188-3033.

In some embodiments, a first fragment, peptide, or polypeptide component of the sp modified dehalogenase herein is present as a fusion protein with a first peptide, polypeptide, or protein of interest. In some embodiments, the first peptide, polypeptide, or protein of interest is selected from the group consisting of an antibody, antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, oligonucleotide probe, peptide nucleic acid, DARPin, anticalin, nanobody, aptamer, affimer, a purified protein, and analyte binding domain(s) of proteins. In some embodiments, the second fragment, peptide, or polypeptide component of the sp modified dehalogenase herein is present as a fusion protein with a second peptide, polypeptide, or protein of interest. In some embodiments, the second peptide, polypeptide, or protein of interest is selected from the group consisting of an antibody, antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, oligonucleotide probe, peptide nucleic acid, DARPin, anticalin, nanobody, aptamer, affimer, a purified protein, and analyte binding domain(s) of proteins. In some embodiments, the first and second peptides, polypeptides, or proteins of interest are interaction elements capable of forming a complex with each other. In some embodiments, the first and second peptides, polypeptides, or proteins of interest are co-localization elements configured to co-localize within a cellular compartment, a cell, a tissue, or an organism. In some embodiments, the second fragment is tethered to a molecule of interest.

In some embodiments, the first and second fragment, peptide, or polypeptide component of a sp modified dehalogenase are fused to antibodies or other binding proteins in order for their proximity to be facilitated by the presence of analyte for the antibodies or other binding proteins (e.g., in a diagnostic assay).

In some embodiments, the first fragment, peptide, or polypeptide component of the sp modified dehalogenase herein and/or the second fragment, peptide, or polypeptide component of the sp modified dehalogenase herein is tethered (directly or via a linker) to a small molecule. In some embodiments, a small molecule tethered to the fragment is capable of interacting (e.g., binding) to a small molecule or other element (e.g., peptide or polypeptide (see above) tethered or fused to the other fragment.

In some embodiments, each fragment of a dehalogenase is tethered (e.g., fused, linked, etc.) to complementary interaction or dimerization elements. In some embodiments, the interaction or dimerization elements facilitate formation of the active dehalogenase complex. For example, a first fragment of dehalogenase is tethered to FRB and a second fragment of dehalogenase is tethered to FKBP. In such an embodiments, the presence of rapamycin induces dimerization of FRB and FKBP and facilitates formation of the dehalogenase complex. In some embodiments, a sp dehalogenase is used in such a system that is not capable of independent active complex formation, but does form an active complex upon facilitation.

In some embodiments, provided herein is a polynucleotide or polynucleotides encoding the split variants described herein. In some embodiments, provided herein is an expression vector or expression vectors comprising the polynucleotide or polynucleotides described herein. In some embodiments, provided herein are host cells comprising the polynucleotide or polynucleotides or the expression vector or expression vectors described herein. In some embodiments, cells are provided in which the genome has been edited to incorporate sequences encoding the split variants described herein.

A split dehalogenase complementation system offers several technical advantages over intact or circularly permuted dehalogenases. While the covalent labeling of intact dehalogenase with chloroalkane ligands can allow direct readouts of the location and concentration of a protein, a split dehalogenase directs such labeling to sites of molecular interactions (e.g., protein-protein interactions). Many critical cellular functions, including signal transduction, transcription, translation, and cargo trafficking require specific interactions between proteins, membranes, organelles, and subcellular structures. A split dehalogenase system reports on the location, timing, and frequency of these events, whereas intact dehalogenase can only report on the presence of the molecules.

In some embodiments, the split dehalogenases systems, compositions, and methods herein find use in fluorescence microscopy and/or imaging applications. For example, split modified dehalogenases allow for monitoring of functional/molecular events (e.g., protein:protein interactions) with the fluorescent ligands beyond cell culture, for example, in live animals, tissues, organoid model systems, etc. split dehalogenases find use in measuring the localization and occurrence of molecular events within subcellular structures, at cell:cell interactions or interfaces, and in deep tissues of live organisms. These uses can further be configured into high-throughput formats for screening or diagnostic applications.

The components of a split dehalogenase individually present reduced activity compared to the active complex assembled therefrom. In some embodiments, assembly of the active complex occurs with the aid of interacting partner proteins fused to each fragment. Bimolecular fluorescence complementation (BiFC) of the green fluorescent protein (GFP) and other FPs has been used by researchers for years, but these BiFC systems have several crucial shortcomings. The fluorophores take time to mature, and the proteins tend to assemble irreversibly and suffer from poor performance in hypoxic conditions. In contrast, experiments conducted during development of embodiments herein demonstrate that some split dehalogenases assemble reversibly, and when coupled with fluorescently-tagged ligands, employ an exogenously-supplied, cell-permeable fluorescent ligand that requires no maturation or oxygen. In some embodiments, provided herein are chloroalkane ligands featuring bright, stable fluorophores that outperform protein-based fluorophores in terms of signal strength (e.g., quantum yield and extinction coefficient) and temporal-spatial resolution (e.g., image resolution), making them ideal for advanced imaging applications such as super-resolution microscopy and light sheet microscopy.

In contrast to other enzymatic complementation-based reporter systems, such as split luciferase, split dehalogenase forms a permanent covalent link with the substrate, creating a durable event mark that can be observed for hours, days, or longer. Although the link with the ligand cannot form in the absence of complementation of the split dehalogenase fragments, the covalent link remains even after the dehalogenase complex disassembles. Moreover, multiple complementation events can lead to signal accumulation that does not diminish as the substrate is depleted. This is in contrast with split luciferase, whose signal diminishes over time.

The utility of split dehalogenase extends beyond fluorescence imaging. Dehalogenase can accept a wide variety of ligands, provided the ligands harbor a haloalkane functional group. The ligand's cargo may include, but is not limited to, a fluorophore, a chromophore, an analyte-sensing complex, an affinity tag (such as biotin), a signal for protein degradation or post-translational modification, a nucleic acid, a peptide, a polypeptide, a chemical inducer of dimerization, or a solid support. As such, in certain embodiments, a split dehalogenase utilizes a cellular event as the initiation signal for color development, activation of a sensor, affinity tagging, proteolysis, DNA/RNA barcoding, crosslinking, dimerization, or assembly onto a support or molecular scaffold. The ultimate functional output of the split dehalogenase is determined by the choice of ligand supplied by the user. The flexibility of the split dehalogenase systems described herein find use in a variety of methods and applications.

In some embodiments, due to the utilities of certain split modified dehalogenases with fluorescence and for the detection of protein:protein interactions, embodiments herein find use in a variety of cell sorting applications. For example:
Sorting for presence of the complemented LgHT:SmHT or “dual” tag (SmHT-HiBiT) during CRISPR cell line generation. This helps solve the problem of how to isolate clonal cell lines that have been edited with the tag without “blind” sorting, which adds significant labor and time to isolating cell lines with a tag. With a sortable tag that enables fluorescent detection, a user can immediately sort edited cells for those with the edit.
Sorting for cells that contain the complemented spHaloTag when expressed from plasmids, typically fused to other proteins.
Sorting cells for those containing (or not containing) a specific PPI. This provides for enrichment for cells containing the interacting proteins in order to enable downstream assays, diagnostics, or purification of cells (such as modified T-cells).
Sorting for cells that have undergone a facilitated molecular interaction or molecular proximity, through a stimulus such as a small molecule or hormone. A specific example is sorting for cells that have formed ternary complexes via treatment with PROTACs, molecular glues, or other “TACs”. Other examples are sorting cells for molecular interactions through BRET and sorting cells that have a difference in fluorescence signal due to target engagement (e.g., for drug screening) that is being detected by the split HaloTag.
Sorting of cells that have been infected by a virus through the viral delivery of a nucleic acid sequence encoding the split HaloTag fragments into the cell or more directly if the viral proteins infecting the cells are themselves comprised of fusions to the spHaloTag components (such as a viral coat protein).
Methods that combine cell imaging and flow cytometry or sorting to simultaneously measure morphological cell characteristics and reporter or dye localization to evaluate cell populations (e.g., diagnostics), identify or isolate rare or difficult to culture cell types, or complex phenotyping. The use of a split dehalogenase with these methods enables, for example, cell cycle analysis, apoptosis detection, immunophenotyping, detection and quantification of intracellular signaling, drug screening, microbial population analysis, and stem cell analysis, among others.

In some embodiments, provided herein are methods to detect a protein-protein interaction in a sample comprising contacting: (a) a first fusion comprising: (i) a first complementary fragment of a split variant of a polypeptide comprising at least 70% (e.g., at least 70%, at least 75%, at least 80%, at least 85%, at least 90%, at least 95%, 100%) sequence similarity with a first portion of SEQ ID NO: 1; and (ii) a first protein of interest; (b) a second fusion comprising: (i) a second complementary fragment of a split variant of a polypeptide comprising at least 70% (e.g., at least 70%, at least 75%, at least 80%, at least 85%, at least 90%, at least 95%, 100%) sequence similarity with a second portion of SEQ ID NO: 1; and (ii) a second protein of interest; and (c) a substrate comprising R-linker-A-X, wherein R is a functional group or solid support, X is a halogen, and A-X is a substrate for a dehalogenase enzyme; wherein binding of the first protein of interest to the second protein of interest results in formation of a complex between the first complementary fragment and the secondary complementary fragment that is capable for forming a covalent bond with the substrate.

In certain embodiments, provided herein are methods to detect an interaction between two proteins in a sample. Methods herein include providing a sample having a cell comprising fusions of first and second heterologous protein sequences and first and second complementary fragments of a split dehalogenase or expression vector(s) of the invention (e.g., encoding complementary fragments of a split dehalogenase), a lysate thereof, or an in vitro transcription/translation reaction comprising such components; and a hydrolase substrate (e.g., haloalkane) with at least one functional group under conditions effective to allow for association of the first and second fusion proteins. The presence, amount, or location of at least one functional group in the sample is detected.

In some embodiments, provided herein are methods to detect an interaction between two proteins in a sample, comprising: (a) expressing within the sample a first fusion comprising: (i) a first complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1; and (ii) a first protein of interest; (b) expressing within the sample a second fusion comprising: (i) a second complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1; and (ii) a second protein of interest; (c) contacting the sample with a substrate comprising R-linker-A-X, wherein R is a functional group or solid support, X is a halogen, and A-X is a substrate for a dehalogenase enzyme; and (d) detecting the presence, amount and/or location of the at least one functional group.

In another embodiment, provided herein are methods to detect a molecule of interest in a sample. The methods include providing a sample comprising a cell comprising the molecule of interest bound to a first complementary fragment of a split dehalogenase and a fusion of a second complementary fragment of a split dehalogenase and a heterologous protein (or expression vector encoding the fusion), a lysate thereof, or an in vitro transcription/translation reaction comprising such components; and a hydrolase substrate (e.g., haloalkane) with at least one functional group under conditions effective to allow the heterologous protein to interact with the molecule of interest in the sample. The presence, amount, or location of at least one functional group in the sample is detected, thereby detecting the presence, amount, or location of the molecule of interest.

In some embodiments, provided herein are methods to detect a molecule of interest in a sample, comprising: (a) contacting the sample with a first complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1 tethered to the molecule of interest; and (b) expressing within the sample or contacting the sample with a fusion comprising: (i) a second complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1; and (ii) a protein capable of binding to the molecule of interest; (c) contacting the sample with a substrate comprising R-linker-A-X, wherein R is a functional group or solid support, X is a halogen, and A-X is a substrate for a dehalogenase enzyme; and (d) detecting the presence, amount and/or location of the at least one functional group.

In some embodiments, provided herein are methods to detect the effect of an agent on the interaction of two proteins, the method comprising: (a) expressing within the sample or contacting the sample with a first fusion comprising: (i) a first complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1; and (ii) a first protein sequence; (b) expressing within the sample or contacting the sample with a fusion comprising: (i) a second complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1; and (ii) a second protein sequence capable of binding to the first protein sequence; (c) contacting the sample with a substrate comprising R-linker-A-X, wherein R is a functional group or solid support, X is a halogen, and A-X is a substrate for a dehalogenase enzyme; and (d) contacting the sample with the agent; (e) detecting the presence, amount and/or location of the at least one functional group.

In some embodiments, provided herein are methods to detect the effect of an agent on the interaction of a protein of interest and a ligand of the protein, the method comprising: (a) expressing within the sample or contacting the sample with a fusion comprising: (i) a first complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1; and (ii) the protein of interest; (b) contacting the sample with a second complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1 tethered to the ligand; (c) contacting the sample with a substrate comprising R-linker-A-X, wherein R is a functional group or solid support, X is a halogen, and A-X is a substrate for a dehalogenase enzyme; and (d) contacting the sample with the agent; (e) detecting the presence, amount and/or location of the at least one functional group.

In some embodiments, provided herein are methods of controllable target protein degradation comprising: (a) providing or expressing in a sample a first fusion comprising: (i) a first complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1; and (ii) the target protein; (b) contacting the sample with a proteolysis targeting chimera (PROTAC) of a haloalkane and a ligand capable of engaging an E3 ubiquitin ligase; (c) contacting the sample with a second complementary fragment of the split variant of a polypeptide comprising at least 70% (e.g., at least 70%, at least 75%, at least 80%, at least 85%, at least 90%, at least 95%, 100%) sequence similarity with a portion of SEQ ID NO: 1, wherein formation of the split variant complex results in binding of the haloalkane by the split variant complex, bringing the ligand capable of engaging an E3 ubiquitin ligase in proximity of the target protein, ubiquitination of the target protein, and directing the target protein for proteasome degradation. In some embodiments, the first fusion further comprises a luciferase or a first component of a bioluminescent complex and one of the complementary fragments is tethered to a fluorophore, wherein light emission from the luciferase or the bioluminescent complex is capable of exciting the fluorophore.

In some embodiments, provided herein are methods of controllable target protein modification comprising: (a) providing or expressing in a sample a first fusion comprising: (i) a first complementary fragment of a split variant of a polypeptide comprising at least 70% (e.g., at least 70%, at least 75%, at least 80%, at least 85%, at least 90%, at least 95%, 100%) sequence similarity with a portion of SEQ ID NO: 1; and (ii) the target protein; (b) contacting the sample with a chimera of a haloalkane and a ligand capable of engaging a protein-modifying enzyme; (c) contacting the sample with a second complementary fragment of the split variant of a polypeptide comprising at least 70% (e.g., at least 70%, at least 75%, at least 80%, at least 85%, at least 90%, at least 95%, 100%) sequence similarity with a portion of SEQ ID NO: 1, wherein formation of the split variant complex results in binding of the haloalkane by the split variant complex, bringing the ligand capable of engaging the protein-modifying enzyme in proximity of the target protein, and modification of the target protein. In some embodiments, the chimera is a PhosTAC, and the protein-modifying enzyme is a phosphatase.

In some embodiments of any of the methods herein, the first complementary fragment comprises at least 70% (e.g., at least 70%, at least 75%, at least 80%, at least 85%, at least 90%, at least 95%, 100%) sequence identity with the first portion of SEQ ID NO: 1 and the second complementary fragment comprises at least 70% (e.g., at least 70%, at least 75%, at least 80%, at least 85%, at least 90%, at least 95%, 100%) sequence identity with the second portion of SEQ ID NO: 1.

In some embodiments of any of the methods herein, the first portion of SEQ ID NO: 1 is selected from SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, and 576; and the second portion of SEQ ID NO: 1 is selected from SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, and 577.

In some embodiments of any of the methods herein, the first complementary fragment comprises at least 70% (e.g., at least 70%, at least 75%, at least 80%, at least 85%, at least 90%, at least 95%, 100%) sequence similarity with one of SEQ ID NOS: 578-1187 (or 100% identity to SEQ ID NOS: 3034 or 3035), and the second complementary fragment comprises at least 70% (e.g., at least 70%, at least 75%, at least 80%, at least 85%, at least 90%, at least 95%, 100%) sequence similarity with one of 1188-3033.

In some embodiments of any of the methods herein, the first complementary fragment comprises at least 70% (e.g., at least 70%, at least 75%, at least 80%, at least 85%, at least 90%, at least 95%, 100%) sequence identity with one of SEQ ID NOS: 578-1187 (or 100% identity to SEQ ID NOS: 3034 or 3035), and the second complementary fragment comprises at least 70% (e.g., at least 70%, at least 75%, at least 80%, at least 85%, at least 90%, at least 95%, 100%) sequence identity with one of 1188-3033.

In certain embodiments, cells, beads, nanoparticles, liposomes, or other structures are provided that display first and/or second complementary fragments of a split dehalogenase (e.g., spHT). In some embodiments, the cell-surface-displayed split dehalogenases find use in bacterial display, yeast display, mammalian display, phage display, etc. In some embodiments, surface-displayed split dehalogenases are free to interact with non-permeable substrates, can be used for detection of analytes in solution, or detect cell-cell interactions if both cells display the complementary split protein fragments.

Also provided herein are methods to detect an agent that alters the interaction of two proteins, which includes providing a sample having a cell comprising fusions of first and second complementary fragments of a split dehalogenase and first and second heterologous proteins (or expression vector(s) encoding the fusions), a lysate thereof, or an in vitro transcription/translation reaction comprising such components; a hydrolase substrate (e.g., haloalkane) with at least one functional group, and an agent under conditions effective to allow for association of the first and second fusion proteins. The agent is suspected of altering the interaction of the first and second heterologous proteins. The presence or amount of at least one functional group in the sample relative to a sample without the agent is detected. In some embodiments, multiple concentrations of the agents are assayed to determine the effect of the agent on the protein-protein interaction. In some embodiments, screens are provided in which a library (e.g., 10, 20, 50, 100, 200, 500, 1000, 2000, 5000, 10,000, 20,000, 100,000, or more) agents and/or heterologous protein sequences are screened using the system herein.

In another embodiment, methods are provided to detect an agent that alters the interaction of a molecule of interest and a protein. The methods include providing a sample comprising a cell comprising the molecule of interest bound to a first complementary fragment of a split dehalogenase and a fusion of a second complementary fragment of a split dehalogenase and a heterologous protein (or expression vector encoding the fusion), a lysate thereof, or an in vitro transcription/translation reaction comprising such components; a hydrolase substrate (e.g., haloalkane) with at least one functional group; and an agent suspected of altering the interaction between the heterologous amino acid sequence and a molecule of interest in the sample, under conditions effective to allow the heterologous protein to interact with the molecule of interest in the sample. The presence or amount of the functional group in the sample relative to a sample with the agent. In some embodiments, multiple concentrations of the agent are assayed to determine the effect of the agent on the protein-protein interaction. In some embodiments, screens are provided in which a library (e.g., 10, 20, 50, 100, 200, 500, 1000, 2000, 5000, 10,000, 20,000, 100,000, or more) of agents, molecules of interest, and/or heterologous protein sequences are screened using the system herein.

In some embodiments, provided herein are methods of detecting the presence of a molecule of interest. For instance, a cell is contacted with vector(s) comprising a promoter, e.g., a regulatable promoter, and a nucleic acid sequence encoding the two complementary fragments of a mutant hydrolase, at least one of which is fused to a protein which interacts with the molecule of interest. In one embodiment, a transfected cell is cultured under conditions in which the promoter induces transient expression of the fragments or regulated expression of one of the fragments and an activity associated with the labeled substrate is detected.

In some embodiments, methods are provided for expressing one or both complementary fragments of a split dehalogenase (e.g., spHT) within a cell. In some embodiments, the split dehalogenase, or a fragment thereof (or a fusion thereof), is transiently expressed by a cell. In some embodiments, a nucleic acid encoding the split dehalogenase or a fragment thereof (or a fusion thereof) is stably incorporated into a cell (or the genome thereof). In some embodiments, provided herein are cells or cell lines that encode and are capable of expressing one or both complementary fragments of a split dehalogenase (e.g., spHT) or a fusion thereof. In some embodiments, methods are provided for generating such cells, for example, by transfection of a nucleic acid vector into the cell and/or through CRISPR insertion of the split dehalogenase (e.g., spHT) construct into the genome of the cell.

Other methods described herein or that are performable with the split dehalogenases herein are within the scope of the present technology.

### BRIEF DESCRIPTION OF THE DRAWINGS

FIG. 1. Enzyme activity, thermal stability, and TEV protease-induced stability changes of circularly permuted HaloTag (“cpHT”) library variants. (A) E. coli lysates containing overexpressed cpHT proteins (position of circular permutation (“cp”) indicated along x-axis) were diluted 5-fold, then mixed 1:1 with CA-Alexa Fluor488 HaloTag ligand to 10 nM final concentration. Fluorescence polarization (FP) was monitored for 30 min, and initial velocities were calculated (ΔmP/s). Relative activity was calculated by dividing the cpHT velocities by that of lysate containing overexpressed 6×His-HaloTag7 control protein. (B) The same lysates (undiluted) were heated to 40-90° C. for 30 min, then cooled to room temperature (25° C.), and mixed 1:1 with CA-TMR to 10 nM final concentration. FP was measured after a 2 h room temperature incubation. The FP intensity is represented by gray shading, with darker shading indicating higher FP values. (C) The experiment in (B) was repeated using lysates treated with TEV protease. Changes in FP compared to the non-TEV-treated lysates are indicated by shading, with white indicating more negative changes and black indicating more positive changes.

FIG. 2. Fold increase in JF646 signal after rapamycin addition to non-overlapping split HaloTag fragments. E. coli lysates containing overexpressed sp HaloTag (“spHT”) protein fragments fused to FRB or FKBP were mixed in the combinations shown on the left of the table. Lysate mixtures were incubated at room temperature for 30 minutes with 50 nM rapamycin (or without rapamycin as a control). 100 nM Janelia Fluor 646 HaloTag ligand (JF646) was added 1:1 (vol) to the mixtures (50 nM final concentration). Samples were incubated for 24 hours at room temperature. Samples were analyzed for fluorescence (excitation: 646 nm, emission: 664 nm) on a Tecan Infinite M1000 microplate reader. Fold signal increase was computed as Fᵣₐₚ₊/Fᵣₐₚ₋ for each combination.

FIG. 3. Fold increase in JF646 signal after rapamycin addition to partially overlapping split HaloTag fragments. Experimental conditions were identical to those in FIG. 2.

FIG. 4. Optimized-gain (179-183) fluorescence (JF646) of spHT FRB/FKBP lysate mixtures pretreated for 24 h with varying concentrations of rapamycin (0-500 nM). Measurements were taken 24 h after JF646 addition to 50 nM (1:1 volume increase), which followed a 24 h pre-incubation with the indicated concentration of rapamycin at room temperature. Fold increase (lower graph) was calculated as the ratio of signal with rapamycin to that without rapamycin.

FIG. 5. JF646 signal blockage by FK506 competitive inhibition. spHT FRB/FKBP lysate mixtures were reacted with 500 nM rapamycin (or buffer) for 24 h at room temperature (same samples as in FIG. 4). Then, 20-fold molar excess of FK506 (or buffer) was added (1:1 volume increase) and incubated for 24 h at room temperature.

FIG. 6. JF646 signal reversal by FK506 competitive inhibition. Samples from FIG. 4 were allowed to react with JF646 for a total of 48 h. Then, FK506 (or buffer) was added in 10-fold molar excess to the 500 nM rapamycin samples (10 ul of 10× solution added to 40 ul samples).

FIG. 7. Gel electrophoresis of TMR-labeled spHT lysate mixtures under various rapamycin/FK506 conditions. Top gels: lysates were pre-incubated with (or without) 500 nM rapamycin for 24 h, then labeled with 5 μM TMR ligand for 24 h. Bottom gels: lysates were pre-incubated with 500 nM rapamycin. Then, lysates were incubated with 20-fold molar excess of FK506 for 24 h (or just buffer). Finally, lysates were incubated with 5 μM TMR ligand for 24 h.

FIG. 8. TMR fluorescence of SDS-PAGE separated spHT 19 lysate mixtures. The intensities of these bands are shown in FIG. 12. The smaller [1-19] fragment lysate is present at 10×, 1.25×, or 0× concentration relative to the larger [20-297] lysate in each group. Lysate mixtures were pre-incubated with 500 nM rapamycin for 30 min prior to TMR addition. TMR labeling was carried out at room temperature for 20 h.

FIG. 9. Band intensities of TMR-labeled spHT 19 lysate mixtures separated by SDS-PAGE (derived from image analysis of FIG. 11). Shading indicates the relative concentration of the [1-19] component, relative to constant [20-297] lysate, in each pair. The key at the right indicates the identities of the FRB and FKBP fusions used in each lysate combination.

FIG. 10. JF646 fluorescence as a function of increasing spHT [1-19] concentration, with spHT [20-297] concentrations held constant. Lysates were pre-incubated with 500 nM rapamycin for 30 min. Fluorescence was measured 19 h after JF646 addition (100 nM final) at a gain of 160.

FIG. 11. Lysate analysis of HeLa cells co-transfected with spHT FRB/FKBP constructs. HeLa cells were co-transfected with equal amounts of pF4Ag plasmids encoding CMV promoter-driven expression of spHT constructs. The constructs were HT(1-145)-FKBP+HT(146-297)-FRB; HT(1-157)-FKBP+HT(158-297)-FRB; and HT(1-195)-FKBP+HT(196-297)-FRB. Cells were also transfected with pF4Ag encoding non-split HaloTag with a 6× histidine tag as a positive control. Untransfected cells were included as a negative control. Lysates were prepared by passive lysis, treated with (or without) 50 nM rapamycin for 30 minutes, then reacted with 10 μM TMR HaloTag ligand for 24 hours. Samples were electrophoresed on SDS-PAGE, then imaged on a Typhoon FLA 9000 gel imager using the built-in Cy3 protocol.

FIG. 12. Live cell labeling with fluorogenic Janelia Fluor HaloTag ligands. Transfected cells described above were transferred to a 96-well plate, and treated with (or without) 50 nM rapamycin for 30 minutes at 37° C. JF646 or JF585 ligand was added (to 200 nM final concentration) to the cells. Cells were incubated at 37° C. for 22 hours. Fluorescence was measured (JF646: 646 nm/664 nm; JF585: 585 nm/609 nm) on a Tecan Infinite M1000 microplate reader. The instrument gain was manually set at 100 to allow direct comparison of the relative brightness of both dyes. Error bars show the standard deviation of three replicate labeling reactions.

FIG. 13. TMR labeled lysates of HeLa cells transfected with HaloTag or spHT plasmids. Cells were also transfected with pF4Ag encoding non-split HaloTag with a 6× histidine tag as a positive control. Untransfected cells were included as a negative control. Lysates were prepared by passive lysis, treated with (or without) 50 nM or 500 nM rapamycin for 30 minutes, then reacted with 10 μM TMR HaloTag ligand for 24 hours. Samples were electrophoresed on SDS-PAGE, then imaged on a Typhoon FLA 9000 gel imager using the built-in Cy3 protocol.

FIG. 14. Fluorescence of live HeLa cells labeled with 200 nM JF646 or JF585 for 18 hr in the presence of 50 nM rapamycin. Error bars show standard deviation of three replicate samples. 6×His-HT7 data are omitted from the graphs to prevent y-axis compression, but are: JF646: 15700±1150AU (rap+) and 14200±2450AU (rap−); JF585: 36100±3160AU (rap+) and 35300±6640AU (rap−).

FIG. 15A-B. Exemplary ‘dual warhead’ haloalkane ligands. FIG. 15A A SNAP-tag ligand linked to a chloroalkane by a suitable linker. FIG. 15B A photocaged TMP ligand capable of binding to E. coli dihydrofolate reductase (DHFR) upon uncaging, linked to a chloroalkane by a suitable linker.

FIG. 16. Complementation of split HaloTag fragments containing internal deletions as fusions to FRB or FKBP. Proteins were expressed separately in E. coli lysates and combined. Complementation of each pair was induced with the addition of 1 uM Rapamycin (left) or PBS (right) for 2 hours at room temperature and then labeled with 10 uM TMR HaloTag ligand prior to resolution by SDS-PAGE and fluorescence detection.

FIG. 17. Complementation with internal split HaloTag fragments containing overlapped and gapped regions as fusions to FRB or FKBP. Proteins were expressed separately in E. coli lysates and combined. Complementation of each pair was induced with the addition of 1 uM Rapamycin (right) or PBS (left) for 2 hours at room temperature and then labeled with 10 uM TMR HaloTag ligand prior to resolution by SDS-PAGE and fluorescence detection.

FIG. 18. Domain-swapping with a full length cpHaloTag D106A mutant restores activity of cpHaloTags internal split fragments. Proteins were expressed separately in E. coli lysates as FRB or FKBP fusions and combined. Complementation of each pair was induced with the addition of 500 nM Rapamycin (right) or PBS (left) for 30 minutes at room temperature and then labeled with 10 uM TMR HaloTag ligand prior to resolution by SDS-PAGE and fluorescence detection. Red boxes indicate detectable TMR labeling of active pairs.

FIG. 19A-B. Complementation and reversibility of spHT-FRB/FKBP constructs with added NanoBiT functionality. Proteins were expressed separately in E. coli lysates as FRB or FKBP fusions with NanoBiT tags and combined. Construct labels represent the boundary of split fragments (i.e., spHT146 was expressed as HT(1-145)-FKBP-SmBiT and HT(146-297)-FRB-LgBiT fragments). Complementation of each pair was induced with the addition of 500 nM Rapamycin for 1 hour. FK506 was added at 5 uM and incubated for 4 hours in order to test reversibility. Each reaction was then tested for FIG. 19A NanoBiT and FIG. 19B JF646 labeling activity after separating reactions. Error bars show standard deviation of duplicate measurements.

FIG. 20A-C. Complementation of split HaloTag fragments in human body fluid matrices. Proteins were expressed separately in E. coli as FRB or FKBP fusions and combined after lysis. To each lysis combination, 0-20% human plasma FIG. 20A, serum FIG. 20B, or urine FIG. 20C followed by Rapamycin (where indicated) was added and incubated for 2 hours at room temperature. Aliquots of each reaction were tested separately for NanoBiT assay luminescence or HaloTag activity by binding of fluorescent JF635 HaloTag ligand. Error bars represent one standard deviation from the means of duplicate reactions.

FIG. 21A-B. Comparison of complementation activity of N-terminal split HaloTag constructs. Each HaloTag fragment was expressed separately in E. coli and then combined after lysis. The smaller N-terminal HaloTag fragments as FKBP fusions were tested against the larger fragments of FIG. 21A HT(23-297)-FRB or FIG. 21B HT(22-297)-FRB. To each reaction, 500 nM Rapamycin was added and incubated at room temperature for 2.5 hours prior to addition of 50 nM JF646 ligand and measurement of fluorescence at the indicated timepoint.

FIG. 22. Comparison of truncations of N-terminal split HaloTag constructs. Each HaloTag fragment was expressed separately in E. coli and then combined after lysis. The smaller N-terminal HaloTag fragments as FKBP fusions were tested against the larger fragments of HT(23-297)-FRB. To each reaction, 500 nM Rapamycin was added and incubated at room temperature for 2 hours prior to addition of 50 nM JF646 ligand and measurement of fluorescence at the indicated timepoint.

FIG. 23A-B. Complementation of N-terminal split HaloTag as fusions to NanoBiT tags. Each HaloTag fragment was expressed separately in E. coli and then combined after lysis. The FKBP-HT(1-33) fragment as SmBiT or HiBiT fragment fusions were tested against HT(23-297)-FRB fragment fused to LgBiT. To each reaction, 500 nM Rapamycin was added and incubated at room temperature for 2 hours prior to separation of the reaction volume for either addition of JF646 ligand and measurement of fluorescence or addition of NanoGlo® assay reagent for luminescence measurement.

FIG. 24A-B. Complementation of N-terminal split HaloTag as fusions to NanoBiT tags. Each HaloTag fragment was expressed separately in E. coli and then combined after lysis. The FKBP-HaloTag fragments as C-terminal HiBiT fusions were tested against the HT(23-297)-FRB fragment. To each reaction, 500 nM Rapamycin was added and incubated at room temperature for 2 hours prior to separation of the reaction volume for either FIG. 21A addition of JF646 ligand and measurement of fluorescence or FIG. 21B addition of purified LgBiT and NanoGlo® assay reagent for luminescence measurement.

FIG. 25A-B. Mutations in N-terminal split HaloTag fragments improve fluorescence intensity and fold response. Each HaloTag fragment was expressed separately in E. coli and then combined after lysis. The smaller N-terminal HaloTag fragments as FKBP fusions were tested against the larger fragments of HT(23-297)-FRB. To each reaction, 500 nM Rapamycin was added and incubated at room temperature for 2 hours prior to addition of 50 nM JF646 ligand and measurement of fluorescence at the indicated timepoint. FIG. 25A Fluorescence intensity is shown for +Rapamycin condition to show the overall system brightness relative to FIG. 25B Fold response following Rapamycin addition.

FIG. 26A-C. Mutations in N-terminal split HaloTag fragments improve fluorescence intensity and fold response with multiple HaloTag ligands. Each HaloTag fragment was expressed separately in E. coli and then combined after lysis. The smaller N-terminal HaloTag fragments as FKBP fusions were tested against the larger fragments of HT(22-297)-FRB or HT(23-297)-FRB. To each reaction, 500 nM Rapamycin was added and incubated at room temperature for 2 hours prior to addition of 50 nM FIG. 26A JF549, FIG. 26B JF635, or FIG. 26C JF646 ligand and measurement of fluorescence at the indicated timepoint. Constructs with the Q165H+P174R mutations are labeled with “+HT9”. Relative brightness was calculated as the fractional brightness compared to a HaloTag7 control.

FIG. 27A-B. Activity of split HaloTag combinations in live mammalian cells. HeLa cells transiently transfected with plasmids expressing the large HaloTag fragments fused to FRB and small HaloTag fragments fused to FKBP were incubated for 2 hours with 500 nM Rapamycin, followed by labeling with 50 nM JF646 HaloTag ligand prior to detection of fluorescence at indicated timepoints. FIG. 27A Fluorescence intensity of JF646 HaloTag ligand in live cell assays over time comparing cells treated or untreated with Rapamycin. FIG. 27B Fold response of each assay condition was calculated as the ratio of fluorescence signal for +Rapamycin/−Rapamycin treated cells.

FIG. 28A-B. Live cell imaging of split HaloTag function in mammalian cells. HeLa cells transiently transfected with FKBP-HT(1-30)+HT(23-297)-FRB were incubated overnight with 1 uM Rapamycin. Prior to imaging by confocal microscopy, cells were labeled with 100 nM JF646 HaloTag ligand and nuclei stained with DAPI. FIG. 28A Image data was collected in Far-red channel (Ex. 637 nm, left) and blue/Far-red/DIC merged channel (Ex. 408 nm, right). FIG. 28B Comparison of quantitated far-red channel fluorescence intensity for cells expressing split HaloTag fragments versus HaloTag7.

FIG. 29A-C. Live cell imaging of split HaloTag complementation activity in mammalian cells. HeLa cells transiently transfected with EGFP-FKBP-HT(1-30)+HT(23-297)-FRB were incubated overnight with or without 1 uM Rapamycin. Prior to imaging by confocal microscopy, cells were labeled with 100 nM JF646 HaloTag ligand. FIG. 29A Image data was collected in Far-red channel (Ex. 637 nm, left) and Green channel (Ex. 488 nm, right) for cells treated with (top) or without (bottom) Rapamycin. Comparison of quantitated far-red and green channel fluorescence intensities for cells expressing split HaloTag fragments relative to the signal from the EGFP fusion to FKBP-HT(1-30) for conditions FIG. 29B with Rapamycin or FIG. 29C without Rapamycin.

FIG. 30. Complementation of HaloTag[22-297](M2F) fragment in E. coli lysates using a synthetic HaloTag[3-19] peptide. HaloTag[22-297](M2F) was expressed in E. coli lysates and combined with indicated amounts of synthetic HaloTag[3-19] peptide. Reactions were incubated for 2 hours at room temperature and then labeled with 50 nM JF646 HaloTag ligand prior to fluorescence detection.

FIG. 31. Complementation of HaloTag[22-297](Q145H+P154R) fragment in E. coli lysates using a synthetic HaloTag[3-19] peptide. HaloTag[22-297](Q145H+P154R) was expressed in E. coli lysates and combined with indicated amounts of synthetic HaloTag[3-19] peptide. Reactions were incubated for 2 hours at room temperature and then labeled with 50 nM JF646 HaloTag ligand prior to fluorescence detection.

FIG. 32. Complementation of HaloTag[22-297](M2F) fragments using a synthetic HaloTag[3-19] peptide in a kinetic labeling assay. HaloTag[22-297](M2F) was expressed in E. coli lysates and combined with indicated amounts of synthetic HaloTag[3-19] peptide. Reactions were incubated for 2 hours at room temperature and then labeled with 10 nM TMR HaloTag ligand while reading fluorescence polarization of the reaction.

FIG. 33. Complementation of HaloTag[22-297](Q145H+P154R) fragment using a synthetic HaloTag[3-19] peptide in a kinetic labeling assay. HaloTag[22-297](Q145H+P154R) was expressed in E. coli lysates and combined with indicated amounts of synthetic HaloTag[3-19] peptide. Reactions were incubated for 2 hours at room temperature and then labeled with 10 nM TMR HaloTag ligand while reading fluorescence polarization of the reaction.

FIG. 34. Complementation of purified 6×His-HaloTag[22-297](M2F) fragment using a synthetic HaloTag[3-19] peptide. Purified 6×His-HaloTag[22-297](M2F) at 80 nM was combined with indicated amounts of synthetic HaloTag[3-19] peptide. Reactions were incubated for 18 hours at room temperature and then labeled with 100 nM JF646 HaloTag ligand prior to fluorescence detection.

FIG. 35. Fold response upon complementation of purified 6×His-HaloTag[22-297](M2F) fragment using a synthetic HaloTag[3-19] peptide. Purified 6×His-HaloTag[22-297](M2F) at 80 nM was combined with indicated amounts of synthetic HaloTag[3-19] peptide. Reactions were incubated for 18 hours at room temperature and then labeled with 100 nM JF646 HaloTag ligand prior to fluorescence detection.

FIG. 36. Complementation of purified 6×His-HaloTag[22-297](M2F) fragment using a variant of synthetic HaloTag[3-19] peptide. Purified 6×His-HaloTag[22-297](M2F) at 80 nM was combined with indicated amounts of synthetic HaloTag[3-19] peptide with two addition N-terminal Arginine residues (RREIGTGFPFDPHYVEVLG). Reactions were incubated for 18 hours at room temperature and then labeled with 100 nM JF646 HaloTag ligand prior to fluorescence detection.

FIG. 37. Fold response upon complementation of purified 6×His-HaloTag[22-297](M2F) fragment using a variant of synthetic HaloTag[3-19] peptide. Purified 6×His-HaloTag[22-297](M2F) at 80 nM was combined with indicated amounts of synthetic HaloTag[3-19] peptide with two addition N-terminal Arginine residues (RREIGTGFPFDPHYVEVLG). Reactions were incubated for 18 hours at room temperature and then labeled with 100 nM JF646 HaloTag ligand prior to fluorescence detection.

FIG. 38. Complementation of purified 6×His-HaloTag[22-297](Q145H+P154R) fragment using a synthetic HaloTag[3-19] peptide. Purified 6×His-HaloTag[22-297](Q145H+P154R) at 80 nM was combined with indicated amounts of synthetic HaloTag[3-19] peptide. Reactions were incubated for 18 hours at room temperature and then labeled with 100 nM JF646 HaloTag ligand prior to fluorescence detection.

FIG. 39. Fold response upon complementation of purified 6×His-HaloTag[22-297](Q145H+P154R) fragment using a synthetic HaloTag[3-19] peptide. Purified 6×His-HaloTag[22-297](Q145H+P154R) at 80 nM was combined with indicated amounts of synthetic HaloTag[3-19] peptide. Reactions were incubated for 18 hours at room temperature and then labeled with 100 nM JF646 HaloTag ligand prior to fluorescence detection.

FIG. 40. Complementation of purified 6×His-HaloTag[22-297](Q145H+P154R) fragments using a variant of synthetic HaloTag[3-19] peptide. Purified 6×His-HaloTag[22-297](Q145H+P154R) at 80 nM was combined with indicated amounts of synthetic HaloTag[3-19] peptide with two addition N-terminal Arginine residues (RREIGTGFPFDPHYVEVLG). Reactions were incubated for 18 hours at room temperature and then labeled with 100 nM JF646 HaloTag ligand prior to fluorescence detection.

FIG. 41. Fold response upon complementation of purified 6×His-HaloTag[22-297](Q145H+P154R) fragment using a variant of synthetic HaloTag[3-19] peptide. Purified 6×His-HaloTag[22-297](Q145H+P154R) at 80 nM was combined with indicated amounts of synthetic HaloTag[3-19] peptide with two addition N-terminal Arginine residues (RREIGTGFPFDPHYVEVLG). Reactions were incubated for 18 hours at room temperature and then labeled with 100 nM JF646 HaloTag ligand prior to fluorescence detection.

FIG. 42. Complementation of purified 6×His-HaloTag[22-297](M2F) fragment using shorter variants of synthetic HaloTag[3-19] peptide. Purified 6×His-HaloTag[22-297](M2F) at 80 nM was combined with indicated amounts of synthetic HaloTag[3-19] peptide and shorter variants comprised of HaloTag[8-19] fragments. Reactions were incubated for 18 hours at room temperature and then labeled with 10 nM TMR HaloTag ligand prior to fluorescence detection.

FIG. 43. Complementation of purified HaloTag[22-297](Q145H+P154R)-6×His fragment using shorter variants of synthetic HaloTag[3-19] peptide. Purified HaloTag[22-297](Q145H+P154R)-6×His at 80 nM was combined with indicated amounts of synthetic HaloTag[3-19] peptide and shorter variants comprised of HaloTag[8-19] fragments. Reactions were incubated for 18 hours at room temperature and then labeled with 10 nM TMR HaloTag ligand prior to fluorescence detection.

FIG. 44. Complementation of split HaloTag fragments as fusions to FRB or FKBP containing all HaloTag[3-19] mutations at position 1. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 45. Complementation of split HaloTag fragments as fusions to FRB or FKBP containing all HaloTag[3-19] mutations at position 2. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 46. Complementation of split HaloTag fragments as fusions to FRB or FKBP containing all HaloTag[3-19] mutations at position 3. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 47. Complementation of split HaloTag fragments as fusions to FRB or FKBP containing all HaloTag[3-19] mutations at position 4. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 48. Complementation of split HaloTag fragments as fusions to FRB or FKBP containing all HaloTag[3-19] mutations at position 5. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 49. Complementation of split HaloTag fragments as fusions to FRB or FKBP containing all HaloTag[3-19] mutations at position 6. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 50. Complementation of split HaloTag fragments as fusions to FRB or FKBP containing all HaloTag[3-19] mutations at position 7. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 51. Complementation of split HaloTag fragments as fusions to FRB or FKBP containing all HaloTag[3-19] mutations at position 8. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 52. Complementation of split HaloTag fragments as fusions to FRB or FKBP containing all HaloTag[3-19] mutations at position 9. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 53. Complementation of split HaloTag fragments as fusions to FRB or FKBP containing all HaloTag[3-19] mutations at position 10. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 54. Complementation of split HaloTag fragments as fusions to FRB or FKBP containing all HaloTag[3-19] mutations at position 11. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 55. Complementation of split HaloTag fragments as fusions to FRB or FKBP containing all HaloTag[3-19] mutations at position 12. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 56. Complementation of split HaloTag fragments as fusions to FRB or FKBP containing all HaloTag[3-19] mutations at position 13. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 57. Complementation of split HaloTag fragments as fusions to FRB or FKBP containing all HaloTag[3-19] mutations at position 14. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 58. Complementation of split HaloTag fragments as fusions to FRB or FKBP containing all HaloTag[3-19] mutations at position 15. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 59. Complementation of split HaloTag fragments as fusions to FRB or FKBP containing all HaloTag[3-19] mutations at position 16. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 60. Complementation of split HaloTag fragments as fusions to FRB or FKBP containing all HaloTag[3-19] mutations at position 17. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 61. Complementation of split HaloTag fragments as fusions to FRB or FKBP containing all HaloTag[3-19] mutations at position 1-17 in the absence of Rapamycin. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 62. Complementation of split HaloTag fragments as fusions to FRB or FKBP containing all HaloTag[3-19] mutations at position 1-17 in the presence of Rapamycin. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 63. Complementation of split HaloTag fragments as fusions to FRB or FKBP containing HaloTag[3-19] double mutation combinations, Set #1. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 64. Complementation of split HaloTag fragments as fusions to FRB or FKBP containing HaloTag[3-19] double mutation combinations, Set #2. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 65. Complementation of split HaloTag fragments as fusions to FRB or FKBP containing HaloTag[3-19] double mutation combinations, Set #3. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 66. Complementation of split HaloTag fragments as fusions to FRB or FKBP containing HaloTag[3-19] triple mutation combinations, Set #1. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 67. Complementation of split HaloTag fragments as fusions to FRB or FKBP containing HaloTag[3-19] triple mutation combinations, Set #2. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 68. Complementation of split HaloTag fragments as fusions to FRB or FKBP containing HaloTag[3-19] triple mutation combinations, Set #3. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 69. Complementation of split HaloTag fragments as fusions to FRB or FKBP containing HaloTag[3-19] triple mutation combinations, Set #4. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 70. Complementation of split HaloTag fragments as fusions to FRB or FKBP containing HaloTag[3-19] with 4-8 mutation combinations, Set #1. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 71. Complementation of split HaloTag fragments as fusions to FRB or FKBP containing HaloTag[3-19] with 4-8 mutation combinations, Set #2. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 72. Complementation of split HaloTag fragments as fusions to FRB or FKBP containing HaloTag[3-19] with 8-14 mutation combinations, Set #1. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 73. Complementation of split HaloTag fragments as fusions to FRB or FKBP containing HaloTag[3-19] with 8-14 mutation combinations, Set #2. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 74. Complementation of split HaloTag fragments as fusions to FRB or FKBP containing HaloTag[3-19] with combinations of 17 mutations. HaloTag[22-297](M2F)-FRB and FKBP-HaloTag[3-19] variants were expressed separately in E. coli lysates and combined. Complementation of each pair was induced with the addition of 500 nM Rapamycin for 2 hours at room temperature and then labeled with 50 nM JF646 HaloTag ligand prior to fluorescence detection.

FIG. 75. Relative fluorescence intensity of HaloTag[22-297](M2F) mutants with synthetic HaloTag[3-19] peptide, Set #1. HaloTag[22-297](M2F)-6×His variants were expressed in E. coli lysates and combined with 31 micromolar synthetic HaloTag[3-19] peptide. Reactions were incubated for 2 hours at room temperature and then labeled with 50 nM JF646 HaloTag ligand prior to fluorescence detection. The fluorescence intensity of mutants was normalized to the intensity of the unmutated HaloTag[22-297](M2F) control.

FIG. 76. Relative fluorescence intensity of HaloTag[22-297](M2F) mutants with synthetic HaloTag[3-19] peptide, Set #2. HaloTag[22-297](M2F)-6×His variants were expressed in E. coli lysates and combined with 31 micromolar synthetic HaloTag[3-19] peptide. Reactions were incubated for 2 hours at room temperature and then labeled with 50 nM JF646 HaloTag ligand prior to fluorescence detection. The fluorescence intensity of mutants was normalized to the intensity of the unmutated HaloTag[22-297](M2F) control.

FIG. 77. Relative fluorescence intensity of HaloTag[22-297](M2F) mutants with synthetic HaloTag[3-19] peptide, Set #3. HaloTag[22-297](M2F)-6×His variants were expressed in E. coli lysates and combined with 31 micromolar synthetic HaloTag[3-19] peptide. Reactions were incubated for 2 hours at room temperature and then labeled with 50 nM JF646 HaloTag ligand prior to fluorescence detection. The fluorescence intensity of mutants was normalized to the intensity of the unmutated HaloTag[22-297](M2F) control.

FIG. 78. Relative improvement in fold response of HaloTag[22-297](M2F) mutants with synthetic HaloTag[3-19] peptide, Set #1. HaloTag[22-297](M2F)-6×His variants were expressed in E. coli lysates and combined with 31 micromolar synthetic HaloTag[3-19] peptide. Reactions were incubated for 2 hours at room temperature and then labeled with 50 nM JF646 HaloTag ligand prior to fluorescence detection. The fold response of mutants was normalized to the fold response of the unmutated HaloTag[22-297](M2F) control.

FIG. 79. Relative improvement in fold response of HaloTag[22-297](M2F) mutants with synthetic HaloTag[3-19] peptide, Set #2. HaloTag[22-297](M2F)-6×His variants were expressed in E. coli lysates and combined with 31 micromolar synthetic HaloTag[3-19] peptide. Reactions were incubated for 2 hours at room temperature and then labeled with 50 nM JF646 HaloTag ligand prior to fluorescence detection. The fold response of mutants was normalized to the fold response of the unmutated HaloTag[22-297](M2F) control.

FIG. 80. Relative improvement in fold response of HaloTag[22-297](M2F) mutants with synthetic HaloTag[3-19] peptide, Set #3. HaloTag[22-297](M2F)-6×His variants were expressed in E. coli lysates and combined with 31 micromolar synthetic HaloTag[3-19] peptide. Reactions were incubated for 2 hours at room temperature and then labeled with 50 nM JF646 HaloTag ligand prior to fluorescence detection. The fold response of mutants was normalized to the fold response of the unmutated HaloTag[22-297](M2F) control.

FIG. 81. Relative fluorescence intensity of HaloTag[22-297](M2F) double mutants with synthetic HaloTag[3-19] peptide. HaloTag[22-297](M2F)-6×His variants were expressed in E. coli lysates and combined with 31 micromolar synthetic HaloTag[3-19] peptide. Reactions were incubated for 2 hours at room temperature and then labeled with 50 nM JF646 HaloTag ligand prior to fluorescence detection. The fluorescence intensity of mutants was normalized to the intensity of the unmutated HaloTag[22-297](M2F) control.

FIG. 82. Relative improvement in fold response of HaloTag[22-297](M2F) double mutants with synthetic HaloTag[3-19] peptide. HaloTag[22-297](M2F)-6×His variants were expressed in E. coli lysates and combined with 31 micromolar synthetic HaloTag[3-19] peptide. Reactions were incubated for 2 hours at room temperature and then labeled with 50 nM JF646 HaloTag ligand prior to fluorescence detection. The fold response of mutants was normalized to the fold response of the unmutated HaloTag[22-297](M2F) control.

FIG. 83. Relative fluorescence intensity of HaloTag[22-297](M2F) variants containing multiple mutations with synthetic HaloTag[3-19] peptide. HaloTag[22-297](M2F)-6×His variants were expressed in E. coli lysates and combined with 31 micromolar synthetic HaloTag[3-19] peptide. Reactions were incubated for 2 hours at room temperature and then labeled with 50 nM JF646 HaloTag ligand prior to fluorescence detection. The fluorescence intensity of mutants was normalized to the intensity of the unmutated HaloTag[22-297](M2F) control.

FIG. 84. Complementation of HaloTag[22-297](M2F) mutants with excess HaloTag[3-19] synthetic peptide, Set #1. HaloTag[22-297](M2F) mutants were expressed in E. coli lysates and combined with 250 micromolar synthetic HaloTag[3-19] peptide to saturate binding. Reactions were incubation at room temperature for 2 hours, labeled with 50 nM JF646 HaloTag ligand, and measured for fluorescence after 60 minutes.

FIG. 85. Complementation of HaloTag[22-297](M2F) mutants with excess HaloTag[3-19] synthetic peptide, Set #2. HaloTag[22-297](M2F) mutants were expressed in E. coli lysates and combined with 250 micromolar synthetic HaloTag[3-19] peptide to saturate binding. Reactions were incubation at room temperature for 2 hours, labeled with 50 nM JF646 HaloTag ligand, and measured for fluorescence after 60 minutes.

FIG. 86. Remaining activity of HaloTag[22-297](M2F) mutants after thermal challenge in the presence of excess HaloTag[3-19] synthetic peptide, Set #1. HaloTag[22-297](M2F) mutants were expressed in E. coli lysates and combined with 250 micromolar synthetic HaloTag[3-19] peptide to saturate binding. Reactions were incubated at room temperature for 30 minutes prior to incubation at 40 C for 10 minutes. After returning to room temperature, reactions were labeled with 50 nM JF646 HaloTag ligand and measured for fluorescence after 60 minutes.

FIG. 87. Remaining activity of HaloTag[22-297](M2F) mutants after thermal challenge in the presence of excess HaloTag[3-19] synthetic peptide, Set #2. HaloTag[22-297](M2F) mutants were expressed in E. coli lysates and combined with 250 micromolar synthetic HaloTag[3-19] peptide to saturate binding. Reactions were incubated at room temperature for 30 minutes prior to incubation at 40 C for 10 minutes. After returning to room temperature, reactions were labeled with 50 nM JF646 HaloTag ligand and measured for fluorescence after 60 minutes.

FIG. 88. Remaining fold response of HaloTag[22-297](M2F) mutants after thermal challenge in the presence of excess HaloTag[3-19] synthetic peptide, Set #1. HaloTag[22-297](M2F) mutants were expressed in E. coli lysates and combined with 250 micromolar synthetic HaloTag[3-19] peptide to saturate binding. Reactions were incubated at room temperature for 30 minutes prior to incubation at 40 C for 10 minutes. After returning to room temperature, reactions were labeled with 50 nM JF646 HaloTag ligand and measured for fluorescence after 60 minutes.

FIG. 89. Remaining fold response of HaloTag[22-297](M2F) mutants after thermal challenge in the presence of excess HaloTag[3-19] synthetic peptide, Set #2. HaloTag[22-297](M2F) mutants were expressed in E. coli lysates and combined with 250 micromolar synthetic HaloTag[3-19] peptide to saturate binding. Reactions were incubated at room temperature for 30 minutes prior to incubation at 40 C for 10 minutes. After returning to room temperature, reactions were labeled with 50 nM JF646 HaloTag ligand and measured for fluorescence after 60 minutes.

FIG. 90. The activity of different small HaloTag fragments in live mammalian cells. HeLa cells transiently transfected with plasmids expressing the large HaloTag fragment fused to FRB and small HaloTag fragments fused to FKBP were incubated for 2 hours with 500 nM Rapamycin, followed by labeling with 50 nM JF646 HaloTag ligand prior to detection of fluorescence activity at indicated time points. Fluorescence intensity of JF646 HaloTag ligand in live cell assays over time comparing cells treated or untreated with Rapamycin.

FIG. 91. Fold response of different small HaloTag fragments in live mammalian cells. HeLa cells transiently transfected with plasmids expressing the large HaloTag fragment fused to FRB and small HaloTag fragments fused to FKBP were incubated for 2 hours with 500 nM Rapamycin, followed by labeling with 50 nM JF646 HaloTag ligand prior to detection of fluorescence activity at indicated time points. The fold response of each assay condition was calculated as the ratio of fluorescence signal for +Rapamycin/−Rapamycin treated cells.

FIG. 92. Complementation of split HaloTag fragments by gel analysis. 50 ul of HeLa cell lysate that had been transiently transfected with plasmids expressing the large HaloTag fragment fused to FRB and small HaloTag fragments fused to FKBP were incubated with 25 ul rapamycin at room temperature for 2 hours to induce HaloTag fragments complementation. The final concentration of rapamycin in each well is 500 nM). Then 10 ul of diluted TMR solution was added to all wells and incubated at room temperature in the dark overnight prior to resolution by SDS-PAGE and fluorescence detection. The final concentration of TMR in each well is 2 micromolar.

FIG. 93. Activity comparison of HaloTag[22-297] variants in a protein complementation assay in live mammalian cells. HeLa cells transiently transfected with plasmids expressing the large HaloTag fragment fused to FRB and small HaloTag fragments fused to FKBP were incubated for 2 hours with 500 nM Rapamycin, followed by labeling with 50 nM JF646 HaloTag ligand prior to detection of fluorescence activity at indicated time points. Fluorescence intensity of JF646 HaloTag ligand in live cell assays over time comparing cells treated or untreated with Rapamycin.

FIG. 94. Fold response comparison of HaloTag[22-297] variants in a protein complementation assay in live mammalian cells. HeLa cells transiently transfected with plasmids expressing the large HaloTag fragment fused to FRB and small HaloTag fragments fused to FKBP were incubated for 2 hours with 500 nM Rapamycin, followed by labeling with 50 nM JF646 HaloTag ligand prior to detection of fluorescence activity at indicated time points. The fold response of each assay condition was calculated as the ratio of fluorescence signal for +Rapamycin/− Rapamycin treated cells.

FIG. 95. Live cell imaging of split HaloTag function in mammalian cells. HeLa cells transiently transfected with plasmids expressing FKBP-HaloTag[1-30] and HaloTag[23-297]-FRB were incubated overnight with 1 micromolar Rapamycin. Prior to imaging by confocal microscopy, cells were labeled with 100 nM JF646 HaloTag ligand and nuclei stained with DAPI. Image data were collected in the Far-red channel (Ex. 637 nm, left) and blue/Far-red/DIC merged channel (Ex. 408 nm, right).

FIG. 96. Quantitation of differences between split HaloTag and HaloTag7 in live cell imaging of mammalian cells. HeLa cells transiently transfected with plasmids expressing FKBP-HaloTag[1-30] and HaloTag[23-297]-FRB were incubated overnight with 1 micromolar Rapamycin. Prior to imaging by confocal microscopy, cells were labeled with 100 nM JF646 HaloTag ligand and nuclei stained with DAPI. Comparison of quantitated far-red channel fluorescence intensity for cells expressing split HaloTag fragments versus HT-7.

FIG. 97. Live cell imaging of split HaloTag function in mammalian cells (second series of field of views). HeLa cells transiently transfected with plasmids expressing FKBP-HaloTagT[1-30] and HaloTagT[23-297]-FRB were incubated overnight with 1 micromolar Rapamycin. Prior to imaging by confocal microscopy, cells were labeled with 100 nM JF646 HaloTag ligand and nuclei stained with DAPI. Image data were collected in the Far-red channel (Ex. 637 nm, left) and blue/Far-red/DIC merged channel (Ex. 408 nm, right).

FIG. 98. Live cell imaging of split HaloTag function in mammalian cells (second series of field of views). HeLa cells transiently transfected with plasmids expressing FKBP-HaloTag[1-30] and HaloTag[23-297]-FRB were incubated overnight with 1 micromolar Rapamycin. Prior to imaging by confocal microscopy, cells were labeled with 100 nM JF646 HaloTag ligand and nuclei stained with DAPI. Comparison of quantitated far-red channel fluorescence intensity for cells expressing split HaloTag fragments versus HT-7.

FIG. 99. Live cell imaging of split HaloTag complementation activity in mammalian cells. HeLa cells transiently transfected with plasmids expressing EGFP-FKBP-HaloTag[1-30] and HaloTag[23-297]-FRB were incubated overnight with 1 micromolar Rapamycin. Prior to imaging by confocal microscopy, cells were labeled with 100 nM JF646 HaloTag ligand. The Imaging data were collected in the Far-red channel (Ex. 637 nm, left) and the green channel (Ex. 488 nm, right). *FOV: Field of view.

FIG. 100. Quantitation of differences in fluorescence intensities in live cell imaging of split HaloTag complementation activity in mammalian cells. HeLa cells transiently transfected with plasmids expressing EGFP-FKBP-HaloTagT[1-30] and HaloTagT[23-297]-FRB were incubated overnight with 1 micromolar Rapamycin. Prior to imaging by confocal microscopy, cells were labeled with 100 nM JF646 HaloTag ligand. Comparison of the fluorescence intensity of Split HT vs. EGFP in far-red and green channels, respectively. *FOV: Field of view.

FIG. 101. Live cell imaging of non-complemented split HaloTag activity in mammalian cells. HeLa cells transiently transfected with plasmids expressing EGFP-FKBP-HaloTag[1-30] and HaloTag[23-297]-FRB in the absence of Rapamycin. Prior to imaging by confocal microscopy, cells were labeled with 100 nM JF646 HaloTag ligand. The Imaging data were collected in the Far-red channel (Ex. 637 nm, left) and the green channel (Ex. 488 nm, right). *FOV: Field of view.

FIG. 102. Quantitation of live cell imaging of non-complemented split HaloTag activity in mammalian cells. HeLa cells transiently transfected with plasmids expressing EGFP-FKBP-HaloTag[1-30] and HaloTag[23-297]-FRB were not incubated Rapamycin. Prior to imaging by confocal microscopy, cells were labeled with 100 nM JF646 HaloTag ligand. Comparison of the fluorescence intensity of non-complemented Split HT vs. EGFP in far-red and green channels, respectively. *FOV: Field of view.

FIG. 103. Comparison of the activity of split HaloTag fragment variant combinations in live mammalian cells using a model interaction system. HeLa cells transiently transfected with plasmids expressing the large HaloTag fragments fused to FRB and small HaloTag fragments fused to FKBP were incubated with 1 micromolar Rapamycin overnight at 37° C. The next day, the cells were labeled with 100 nM JF646 HaloTag ligand before detecting fluorescence activity at indicated time points. Fluorescence intensity of JF646 HaloTag ligand in live cell assays over time comparing cells treated or untreated with Rapamycin.

FIG. 104. Comparison of the fold response of split HaloTag fragment variant combinations in live mammalian cells using a model interaction system. HeLa cells transiently transfected with plasmids expressing the large HaloTag fragments fused to FRB and small HaloTag fragments fused to FKBP were incubated with 1 micromolar Rapamycin overnight at 37° C. The next day, the cells were labeled with 100 nM JF646 HaloTag ligand before detecting fluorescence activity at indicated time points. The fold response of each assay condition was calculated as the ratio of fluorescence signal for +Rapamycin/− Rapamycin treated cells.

FIG. 105. Live cell imaging of split HaloTag activity in mammalian cells using a model protein interaction system showing dependence on interaction facilitation for labeling. HeLa cells transiently transfected with plasmids expressing both EGFP-FKBP-HaloTag[3-19] and HaloTag[22-297](Q145H+P154R)-FRB were imaged in +/−Rapamycin conditions; in +Rapamycin condition cells were incubated with 1 micromolar Rapamycin overnight at 37° C. Prior to imaging by confocal microscopy, cells were labeled with 100 nM JF646 HaloTag ligand for 30 minutes at 37° C. The Imaging data were collected in the Far-red channel (Ex. 637 nm, left) and the green channel (Ex. 488 nm, right).

FIG. 106. Quantitation of live cell imaging of split HaloTag activity in mammalian cells using a model protein interaction system. HeLa cells transiently transfected with plasmids expressing both EGFP-FKBP-HaloTag[3-19] and HaloTag[22-297](Q145H+P154R)-FRB were imaged in +/−Rapamycin conditions; in +Rapamycin condition cells were incubated with 1 micromolar Rapamycin overnight at 37° C. Prior to imaging by confocal microscopy, cells were labeled with 100 nM JF646 HaloTag ligand for 30 minutes at 37° C. Comparison of the fluorescence intensity of non-complemented vs. complemented split HaloTag in +/−RAP conditions in the far-red channel, respectively.

FIG. 107. Live cell imaging of split HaloTag activity in mammalian cells using a model protein interaction system showing dependence on interaction facilitation for labeling. HeLa cells transiently transfected with plasmids expressing both EGFP-FKBP-HaloTag[3-19] and HaloTag[22-297](M2F)-FRB were imaged in +/−Rapamycin conditions; in +Rapamycin condition cells were incubated with 1 micromolar Rapamycin overnight at 37° C. Prior to imaging by confocal microscopy, cells were labeled with 100 nM JF646 HaloTag ligand for 30 minutes at 37° C. The Imaging data were collected in the Far-red channel (Ex. 637 nm, left) and the green channel (Ex. 488 nm, right).

FIG. 108. Quantitation of live cell imaging of split HaloTag activity in mammalian cells using a model protein interaction system. HeLa cells transiently transfected with plasmids expressing both EGFP-FKBP-HaloTag[3-19] and HaloTag[22-297](M2F)-FRB were imaged in +/−Rapamycin conditions; in +Rapamycin condition cells were incubated with 1 micromolar Rapamycin overnight at 37° C. Prior to imaging by confocal microscopy, cells were labeled with 100 nM JF646 HaloTag ligand for 30 minutes at Comparison of the fluorescence intensity of non-complemented vs. complemented split HaloTag in +/−RAP conditions in the far-red channel, respectively.

FIG. 109. Comparison of the fluorescent intensity of all imaged cells in serval fields of view in +/−RAP conditions. Each dot represents the intensity of an imaged cell as quantitated using CellProfiler software. The horizontal line is indicative of the median of the data.

FIG. 110. Live cell imaging of split HaloTag activity in mammalian cells using JF585 HaloTag ligand in the presence of facilitated interaction between split HaloTag fragments. HeLa cells transiently transfected with plasmids expressing both EGFP-FKBP-HaloTag[3-19]-3NLS and HaloTag[22-297](Q145H+P154R)-FRB-3NLS were imaged in +Rapamycin condition; in +Rapamycin condition cells were incubated with 1 micromolar Rapamycin overnight at 37° C. Prior to imaging by confocal microscopy, cells were labeled with 100 nM JF585 HaloTag ligand for 30 minutes at 37° C. The Imaging data were collected in the red channel (Ex. 561 nm, left) and the green channel (Ex. 488 nm, right). * NLS: Nuclear Localization Signals.

FIG. 111. Live cell imaging of split HaloTag activity in mammalian cells using JF585 HaloTag ligand in the absence of facilitated interaction between split HaloTag fragments. HeLa cells transiently transfected with both EGFP-FKBP-HaloTag[3-19]-3NLS and HaloTag[22-297](Q145H+P154R)-FRB-3NLS were imaged without the addition of Rapamycin. Prior to imaging by confocal microscopy, cells were labeled with 100 nM JF585 HaloTag ligand for 30 minutes at 37° C. The Imaging data were collected in the red channel (Ex. 561 nm, left) and the green channel (Ex. 488 nm, right). * NLS: Nuclear Localization Signals.

FIG. 112. Live cell imaging of split HaloTag activity in mammalian cells using JF635 HaloTag ligand in the presence of facilitated interaction between split HaloTag fragments. HeLa cells transiently transfected with plasmids expressing both EGFP-FKBP-HaloTag[3-19]-3NLS and HaloTag[22-297](Q145H+P154R)-FRB-3NLS were imaged in +Rapamycin condition; in +Rapamycin condition cells were incubated with 1 micromolar Rapamycin overnight at 37° C. Prior to imaging by confocal microscopy, cells were labeled with 100 nM JF635 HaloTag ligand for 30 minutes at 37° C. The Imaging data were collected in the far-red channel (Ex. 637 nm, left) and the green channel (Ex. 488 nm, right). * NLS: Nuclear Localization Signals.

FIG. 113. Live cell imaging of split HaloTag activity in mammalian cells using JF635 HaloTag ligand in the absence of facilitated interaction between split HaloTag fragments. HeLa cells transiently transfected with plasmids expressing both EGFP-FKBP-HaloTag[3-19]-3NLS and HaloTag[22-297](Q145H+P154R)-FRB-3NLS were imaged in −Rapamycin condition. Prior to imaging by confocal microscopy, cells were labeled with 100 nM JF635 HaloTag ligand for 30 minutes at 37° C. The Imaging data were collected in the far-red channel (Ex. 637 nm, left) and the green channel (Ex. 488 nm, right). * NLS: Nuclear Localization Signals.

FIG. 114. Comparison of the fluorescent intensity of all imaged cells in serval fields of view in +/−RAP conditions with fluorogenic ligand JF585 and JF635. Each dot represents the intensity of an imaged cell. CellProfiler software is used for this analysis. The horizontal line is indicative of the median of the data.

FIG. 115. Live cell imaging of split HaloTag activity in mammalian cells. HeLa cells transiently transfected with plasmids expressing both EGFP-FKBP-HaloTag[3-19] and HaloTag[22-297](Q145H+P154R)-FRB were imaged in +Rapamycin condition, 1 micromolar Rapamycin overnight incubation at 37° C. Prior to imaging by confocal microscopy, cells were labeled with 100 nM JF646 HaloTag ligand for 30 minutes at 37° C. The Imaging data were collected in the Far-red channel (Ex. 637 nm, bottom) and the green channel (Ex. 488 nm, top).

FIG. 116. Live cell imaging of split HaloTag activity in mammalian cells. HeLa cells transiently transfected with plasmids expressing both EGFP-FKBP-HaloTag[3-19] and HaloTag[22-297](Q145H+P154R)-FRB were imaged in and −Rapamycin condition. Prior to imaging by confocal microscopy, cells were labeled with 100 nM JF646 HaloTag ligand for 30 minutes at 37° C. The Imaging data were collected in the Far-red channel (Ex. 637 nm, bottom) and the green channel (Ex. 488 nm, top).

FIG. 117. Live cell imaging of split HaloTag activity in mammalian cells. To measure the background originating from labeling the Large HaloTag fragment, HaloTag[22-297](Q145H+P154R), cells were transfected with just the HaloTag[22-297](Q145H+P154R)-FRB plasmid and imaged in the green channel and the far-red channel. Prior to imaging by confocal microscopy, cells were labeled with 100 nM JF646 HaloTag ligand for 30 minutes at 37° C. The Imaging data were collected in the Far-red channel (Ex. 637 nm, bottom) and the green channel (Ex. 488 nm, top).

FIG. 118. Live cell imaging of split HaloTag activity in mammalian cells. Comparison of the fluorescent intensity of all imaged cells in serval fields of view in +/−rapamycin conditions plus the fluorescent intensity of the labeled non-complemented Large HaloTag fragment. Each dot represents the intensity of an imaged cell. CellProfiler software is used for this analysis. The horizontal line is indicative of the median of the data.

FIG. 119. Live cell imaging of split HaloTag activity in mammalian cells. HeLa cells transiently transfected with plasmids expressing both EGFP-FKBP-HaloTag[3-19] and HaloTag[22-297](M2F)-FRB were imaged in +Rapamycin condition, 1 micromolar Rapamycin overnight incubation at 37° C. Prior to imaging by confocal microscopy, cells were labeled with 100 nM JF646 HaloTag ligand for 30 minutes at 37° C. The Imaging data were collected in the Far-red channel (Ex. 637 nm, bottom) and the green channel (Ex. 488 nm, top).

FIG. 120. Live cell imaging of split HaloTag activity in mammalian cells. HeLa cells transiently transfected with plasmids expressing both EGFP-FKBP-HaloTag[3-19] and HaloTag[22-297](M2F)-FRB were imaged in and −Rapamycin condition. Prior to imaging by confocal microscopy, cells were labeled with 100 nM JF646 HaloTag ligand for 30 minutes at 37° C. The Imaging data were collected in the Far-red channel (Ex. 637 nm, bottom) and the green channel (Ex. 488 nm, top).

FIG. 121. Live cell imaging of split HaloTag activity in mammalian cells. To measure the background originating from labeling the Large HaloTag fragment, HaloTag[22-297](M2F), cells were transfected with just the HaloTag[22-297](M2F)-FRB plasmid and imaged in the green channel and the far-red channel. Prior to imaging by confocal microscopy, cells were labeled with 100 nM JF646 HaloTag ligand for 30 minutes at 37° C. The Imaging data were collected in the Far-red channel (Ex. 637 nm, bottom) and the green channel (Ex. 488 nm, top).

FIG. 122. Time-lapse live cell imaging of split HaloTag complementation and labeling upon the addition of Rapamycin and JF646 in live mammalian cells. HeLa cells transiently transfected with plasmids expressing both EGFP-FKBP-HaloTag[3-19] and HaloTag[22-297](M2F)-FRB were imaged 48 hours post-transfection. The cells were treated with a mixture of 1 micromolar Rapamycin plus 100 nM JF646 and immediately imaged every 15 minutes for 12 hours. The top row of images shows the detection of JF646 HaloTag ligand fluorescence in the far-red channel (Ex. 637 nm), and the bottom row shows the detection of EGFP signal in the green channel (Ex. 488 nm).

FIG. 123. Quantitation of time-lapse live cell imaging of complemented split HaloTag labeling upon the addition of JF646 HaloTag ligand to live mammalian cells. The average of all cell intensities present in the captured fields of view were tracked in the far-red channel over this period.

FIG. 124. Comparing the expression of HaloTag[22-297](Q145H+P154R) and HaloTag[22-297](M2F) when complemented with the small HaloTag fragment in mammalian cells. 50 ul of HeLa cells lysate that was transiently transfected with plasmids expressing EGFP-FKBP-HaloTag[3-19], HaloTag[22-297](Q145H+P154R)-FRB or HaloTag[22-297](M2F)-FRB plasmids and were incubated with 500 nM Rapamycin at room temperature for 2 hours to induce HaloTag fragments complementation. TMR HaloTag ligand at 2 micromolar was added to all wells and incubated at room temperature in the dark overnight prior to resolution by SDS-PAGE and fluorescence detection.

FIG. 125. Use of split HaloTag in detecting the interaction between BRD4 and Histone H3.3 in live mammalian cells. HeLa cells transiently transfected with plasmids expressing HaloTag[22-297](M2F) fused to Histone (H3.3) and EGFP in different orientations, and HaloTag[3-19] fused to C or N-terminus of the BRD4 protein were incubated at 37° C. for 48 hours post transfection. Then, the cells were labeled with 100 nM JF646 HaloTag ligand before detection of fluorescence activity at indicated time points.

FIG. 126. Reversibility measured with split HaloTag of the BRD4:Histone H3.3 interaction in live mammalian cells. HeLa cells transiently transfected with plasmids expressing HaloTag[22-297](M2F), Histone (H3.3), and EGFP in different orientations, and HaloTag[3-19] fused to the C- or N-terminus of BRD4 were incubated at 37° C. for 48 hours post transfection. Cells were incubated with 20 micromolar JQ1, an inhibitor of the interaction, for 24 hours. Cells were labeled with 100 nM JF646 HaloTag ligand before detecting fluorescence activity at indicated time points. For each construct, four technical replicates were tested. The bar for each construct is the mean of the four replicates, and the error bar represents the standard deviation.

FIG. 127. Fold response to inhibitor measured with split HaloTag of the BRD4:Histone H3.3 interaction in live mammalian cells. HeLa cells transiently transfected with plasmids expressing HaloTag[22-297](M2F), Histone (H3.3), and EGFP in different orientations, and HaloTag[3-19] fused to the C- or N-terminus of BRD4 were incubated at 37° C. for 48 hours post transfection. Cells were incubated with 20 micromolar JQ1, an inhibitor of the interaction, for 24 hours. Cells were labeled with 100 nM JF646 HaloTag ligand before detecting fluorescence activity at indicated time points. The fold response for each construct was calculated as the ratio of the fluorescence signal for −JQ1/+JQ1 treated cells. For each construct, four technical replicates have been tested. The bar for each construct is the Mean of the four replicates, and the error bar represents the standard deviation.

FIG. 128. Live cell imaging of split HaloTag detection of the BRD4:Histone H3 interaction in live mammalian cells. HeLa cells transiently transfected with plasmids expressing both BRD4-HaloTag[3-19] and H3.3-HaloTag[22-297](M2F)-EGFP were imaged without the BRD4 inhibitor (JQ1). Prior to imaging by confocal microscopy, cells were labeled with 100 nM JF646 HaloTag ligand for 30 minutes at 37° C. The Imaging data were collected in the far-red channel (Ex. 637 nm) and the green channel (Ex. 488 nm).

FIG. 129. Live cell imaging of split HaloTag detection of inhibition of the BRD4:Histone H3 interaction in live mammalian cells. HeLa cells transiently transfected with plasmids expressing both BRD4-HaloTag[3-19] and H3.3-HaloTag[22-297](M2F)-EGFP were imaged after treatment with 20 micromolar JQ1 inhibitor overnight at 37° C. Prior to imaging by confocal microscopy, cells were labeled with 100 nM JF646 HaloTag ligand for 30 minutes at 37° C. The imaging data were collected in the far-red channel (Ex. 637 nm) and the green channel (Ex. 488 nm). * DIC: Differential Interference Contrast.

FIG. 130. Background measurement of HaloTag[22-297](M2F) fused to Histone H3 in live mammalian cells. To measure the background originating from labeling the HaloTag[22-297](M2F) fragment, cells were transfected with just the H3.3-HaloTag[22-297](M2F)-EGFP plasmid and imaged using the same microscope settings. Prior to imaging by confocal microscopy, cells were labeled with 100 nM JF646 HaloTag ligand for 30 minutes at 37° C. The imaging data were collected in the far-red channel (Ex. 637 nm) and the green channel (Ex. 488 nm).

FIG. 131. Quantitation of live cell imaging of the BRD4:Histone H3 interaction in live mammalian cells using split HaloTag. Comparison of the fluorescent intensity of all imaged cells across several fields of view in the presence or absence of 20 micromolar JQ1 inhibitor and controls labeling the cells expressing the HaloTag[2-297](M2F) fragment alone. Each dot represents the intensity of an imaged cell. CellProfiler software is used for analysis. The horizontal line in each set indicates the median of the data.

FIG. 132. A second independent live cell imaging experiment of split HaloTag detection of the BRD4:Histone H3 interaction in live mammalian cells. HeLa cells transiently transfected with plasmids expressing both BRD4-HaloTag[3-19] and H3.3-HaloTag[22-297](M2F)-EGFP were imaged without the BRD4 inhibitor (JQ1). Prior to imaging by confocal microscopy, cells were labeled with 100 nM JF646 HaloTag ligand for 30 minutes at 37° C. The Imaging data were collected in the far-red channel (Ex. 637 nm) and the green channel (Ex. 488 nm).

FIG. 133 Live cell imaging of split HaloTag detection of inhibition of the BRD4:Histone H3 interaction in live mammalian cells at lower inhibitor concentration. HeLa cells transiently transfected with plasmids expressing both BRD4-HaloTag[3-19] and H3.3-HaloTag[22-297](M2F)-EGFP were imaged after treatment with 20 micromolar JQ1 inhibitor overnight at 37° C. Prior to imaging by confocal microscopy, cells were labeled with 100 nM JF646 HaloTag ligand for 30 minutes at 37° C. The imaging data were collected in the far-red channel (Ex. 637 nm) and the green channel (Ex. 488 nm). * DIC: Differential Interference Contrast.

FIG. 134. Background measurement of HaloTag[22-297](M2F) fused to Histone H3 in live mammalian cells at lower laser intensity. To measure the background originating from labeling the HaloTag [22-297](M2F) fragment, cells were transfected with just the H3.3-HaloTag[22-297](M2F)-EGFP plasmid and imaged using the same microscope settings but lower gain intensity compared to the first set of imaging experiments. Prior to imaging by confocal microscopy, cells were labeled with 100 nM JF646 HaloTag ligand for 30 minutes at 37° C. The imaging data were collected in the far-red channel (Ex. 637 nm) and the green channel (Ex. 488 nm).

FIG. 135. Quantitation of live cell imaging of split HaloTag function in detecting the BRD4 and Histone proteins interaction in live mammalian cells. Comparison of the fluorescent intensity of all imaged cells in serval fields of view in +/−JQ1, 10 micromolar, conditions plus the fluorescent intensity of the labeled non-complemented Large HaloTag fragment. Each dot represents the intensity of an imaged cell. CellProfiler software is used for this analysis. The horizontal line is indicative of the median of the data.

FIG. 136. Timepoint imaging of complemented BRD4:Histone H3 complexes in live cells after addition of JF646 HaloTag ligand. HeLa cells transiently transfected with plasmids expressing BRD4-HaloTag[3-19] and H3.3-HaloTag[22-297](M2F)-EGFP imaged 48 hours post-transfection. Cells were immediately imaged after the ligand, 100 nM JF646, addition every 15 minutes for 1.5 hour. Imaging data were collected in the Far-red channel (Ex. 637 nm, top panel) and the green channel (Ex. 488 nm, bottom panel). Data is shown for the Far-red channel to demonstrate labeling changes over time with the JF646 HaloTag ligand.

FIG. 137. Quantitation of live cell labeling kinetics of split HaloTag fragments fused to BRD4 and Histone H3 using time-lapse imaging. Cells were immediately imaged after the ligand addition every 10 minutes for 70 minutes. A Z-stack image was obtained at all time points to ensure all cells were captured in focus. The most focused Z levels were merged into one, and the intensity of all cells (6 total objects) was measured and averaged at all time points. The average of all cells' intensities present in the captured field of view were tracked in the far-red channel and the green channel over this period.

FIG. 138. Live cell time-lapse imaging of split HaloTag activity as the BRD4 and Histone and so the small HaloTag and Large dissociates upon the addition of BRD4 inhibitor, JQ1. HeLa cells transiently transfected with plasmids expressing both BRD4-HaloTag[3-19] and H3.3-HaloTag[22-297](M2F)-EGFP has imaged 48 hours post-transfection while being labeled with 100 nM JF646 (30 minutes incubation with JF646 before imaging). Then, the cells were treated with 20 micromolar of the BRD4 inhibitor, JQ1, and imaged every 15 minutes immediately after adding JQ1 for 12 hours.

FIG. 139. Quantitation of single live cell time-lapse imaging of inhibition of the BRD4:Histone H3 interaction using split HaloTag fluorescence. HeLa cells transiently transfected with plasmids expressing both BRD4-HaloTag[3-19] and H3.3-HaloTag[22-297](M2F)-EGFP has imaged 48 hours post-transfection while being labeled with 100 nM JF646 (30 minutes incubation with JF646 before imaging). Then, the cells were treated with 20 micromolar of the BRD4 inhibitor, JQ1, and imaged every 15 minutes immediately after adding JQ1 for 12 hours. The intensity of a single cell in both the green and the far-red channel plus its occupied area was tracked over this period.

FIG. 140. Use of split HaloTag in detecting the interaction between Calmodulin and M13 peptide induced by the Calcium ions in live mammalian cells. HeLa cells transiently transfected with plasmids expressing M13-HaloTag[22-297](M2F)-EGFP and HaloTag[3-19]-CaM plasmids were incubated at 37° C. for 48 hours post-transfection. Cells were treated with a mixture of different concentrations of Calcium chloride and 100 nM JF646. The fluorescence activity was measured at indicated time points. For each construct, four technical replicates were tested. The bar for each construct is the mean of the four replicates, and the error bar represents the standard deviations.

FIG. 141. The fold response of split HaloTag in detecting the interaction between Calmodulin and M13 peptide induced by the Calcium ions in live mammalian cells. The fold response of each assay condition was calculated as the ratio of fluorescence signal for +Calcium chloride divided by −Calcium chloride treated cells (B). For each construct, four technical replicates have been tested. The bar for each construct is the mean of the four replicates, and the error bar represents the standard deviations.

FIG. 142. Live cell imaging of split HaloTag function in detecting the interaction between Calmodulin protein with the M13 peptide induced upon the addition of Ca ions in live mammalian cells. HeLa cells transiently transfected with plasmids expressing both with M13-HaloTag[22-297](M2F)-EGFP and HaloTag[3-19]-CaM were imaged in in the presence or absence of 6 mM Calcium chloride conditions 30 minutes after addition at 37° C. Prior to imaging by confocal microscopy, cells were labeled with 100 nM JF646 HaloTag ligand for 30 minutes at 37° C. The imaging data were collected in the far-red channel (Ex. 637 nm) and the green channel (Ex. 488 nm).

FIG. 143. Background measurement of HaloTag[22-297](M2F) fused to M13 peptide in live mammalian cells at lower laser intensity To measure the background originating from labeling the HaloTag [22-297](M2F) fragment, cells were transfected with just the M13-HaloTag[22-297](M2F)-EGFP plasmid and imaged using the same microscope settings. The Imaging data were collected in the far-red channel (Ex. 637 nm) and the green channel (Ex. 488 nm).

FIG. 144. Quantitation of live cell imaging of split HaloTag function in detecting the Calmodulin and M13 peptide interaction in live mammalian cells. Comparison of the fluorescent intensity of all imaged cells across several fields of view in the presence or absence of Calcium chloride (6 mM) conditions compared against the background fluorescent intensity of the labeled non-complemented HaloTag[22-297](M2F) fragment alone. Each dot represents the intensity of an imaged cell. CellProfiler software is used for this analysis. The horizontal line is indicative of the median of the data.

FIG. 145. Use of split HaloTag to detect the interaction between the E3 ligase CRBN and target protein BRD4 upon the addition of the dBET6 PROTAC ligand in live mammalian cells. HeLa cells transiently transfected with plasmids expressing HaloTag[22-297](Q145H+P154R)-EGFP and HaloTag[3-19]-BRD4 plasmids were incubated at 37° C. for 48 hours post-transfection. Cells were treated with a mixture of different concentrations of the PROTAC ligand (dBET6), and +/−10 micromolar MG-132, a proteasome inhibitor, and incubated at 37° C. for two hours. Then, 100 nM JF646 was added to cells, and the fluorescence activity was measured at indicated time points.

FIG. 146. Live cell imaging using split HaloTag to detect ternary complex formation of E3 ligase VHL and target protein BRD4 upon the addition of the MZ1 PROTAC in live mammalian cells. HeLa cells transiently transfected with plasmids expressing both HaloTag[22-297](Q145H+P154R)-VHL-EGFP and HaloTag[3-19]-BRD4 plasmids were imaged after MZ1 addition; cells were incubated with 2 micromolar PROTAC ligand for 2 hours at 37° C. Prior to imaging by confocal microscopy, cells were labeled with 100 nM JF646 HaloTag ligand for 30 minutes at 37° C. For all imaging experiments, the cells were incubated with 10 micromolar MG-132, a proteasome inhibitor, for 2 hours at 37° C. to prevent the possibility of the formed PROTAC ternary complex degradation. The imaging data were collected in the far-red channel (Ex. 637 nm) and the green channel (Ex. 488 nm).

FIG. 147. Background levels of live cell imaging using split HaloTag to detect ternary complex formation of E3 ligase VHL and target protein BRD4 in the absence of the MZ1 PROTAC in live mammalian cells. HeLa cells transiently transfected with plasmids expressing both HaloTag[22-297](Q145H+P154R)-VHL-EGFP and HaloTag[3-19]-BRD4 plasmids were imaged in the absence of MZ1 addition. Prior to imaging by confocal microscopy, cells were labeled with 100 nM JF646 HaloTag ligand for 30 minutes at 37° C. For all imaging experiments, the cells were incubated with 10 micromolar MG-132, a proteasome inhibitor, for 2 hours at 37° C. to prevent the possibility of the formed PROTAC ternary complex degradation. The imaging data were collected in the far-red channel (Ex. 637 nm) and the green channel (Ex. 488 nm).

FIG. 148. Background levels of live cell imaging using split HaloTag to detect ternary complex formation of E3 ligase VHL and target protein BRD4 in the absence of the HaloTag[3-19] fragment in live mammalian cells. To measure the background originating from labeling the HaloTag[22-297](Q145H+P154R) fragment, cells were transfected with just the HaloTag[22-297](Q145H+P154R)-VHL-EGFP plasmid and imaged using the same microscope settings. Prior to imaging by confocal microscopy, cells were labeled with 100 nM JF646 HaloTag ligand for 30 minutes at 37° C. For all imaging experiments, the cells were incubated with 10 micromolar MG-132, a proteasome inhibitor, for 2 hours at 37° C. to prevent the possibility of the formed PROTAC ternary complex degradation. The imaging data were collected in the far-red channel (Ex. 637 nm) and the green channel (Ex. 488 nm).

FIG. 149. Quantitation of live cell imaging using split HaloTag to detect ternary complex formation of E3 ligase VHL and target protein BRD4 in the absence of the MZ1 PROTAC in live mammalian cells. Comparison of the fluorescent intensity of all imaged cells in several fields of view in +/−dBET6 (left) and +/−MZ1 (right), 2 micromolar, conditions plus the fluorescent intensity of the labeled non-complemented large HaloTag fragment. Each dot represents the intensity of an imaged cell. CellProfiler software is used for this analysis. The horizontal line is indicative of the median of the data.

FIG. 150. Live cell imaging using split HaloTag to detect the interaction between endogenous BRD4 and a transiently expressed Histone H3. HeLa cell line edited with CRISPR to express endogenous BRD4 protein tagged with a dual tag, HaloTag[3-19]-HiBiT, was transiently transfected with a plasmid expressing Histone H3.3-HaloTag[22-297](M2F)-EGFP and imaged 48 hours post-transfection. Prior to imaging by confocal microscopy, cells were labeled with 100 nM JF646 HaloTag ligand for 1 hour at 37° C. The imaging data were collected in the far-red channel (Ex. 637 nm) and the green channel (Ex. 488 nm).

FIG. 151. Background levels of live cell imaging using split HaloTag when only when transiently expressing HaloTag[22-297](M2F) fused to Histone H3. To measure the background originating from labeling of transiently expressed HaloTag[22-297](M2F) in the absence of the HaloTag[3-19] fragment, cells were transfected with just the H3.3-HaloTag[22-297](M2F)-EGFP plasmid and imaged using the same microscope setting. Prior to imaging by confocal microscopy, cells were labeled with 100 nM JF646 HaloTag ligand for 1 hour at 37° C. The imaging data were collected in the far-red channel (Ex. 637 nm) and the green channel (Ex. 488 nm).

FIG. 152. Live cell imaging using split HaloTag to detect the interaction between endogenous BRD4 and a transiently expressed VHL E3 ligase in a ternary complex formed with MZ1 PROTAC ligand. HeLa cell line edited with CRISPR to express endogenous BRD4 protein tagged with a dual tag, HaloTag[3-19]-HiBiT, was transiently transfected with a plasmid expressing HaloTag[22-297](Q145H+P154R)-VHL-EGFP. Cells were incubated with 2 micromolar MZ1 PROTAC ligand for 2 hours at 37° C. and then imaged at 48 hours post-transfection. Prior to imaging by confocal microscopy, cells were labeled with 100 nM JF646 HaloTag ligand for 1 hour at 37° C. The imaging data were collected in the far-red channel (Ex. 637 nm) and the green channel (Ex. 488 nm).

FIG. 153. Improved expression of HaloTag[22-297](M2F) following introduction of mutations. HeLa cells transiently transfected with plasmids expressing different mutants of HaloTag[22-297](M2F)-HiBiT were incubated at 37° C. for about 48 hours post-transfection. Bioluminescence signal was measured after cell lysis by addition of LgBiT and luminescent substrate (Furimazine). The bioluminescence activities are normalized to the activity of the unmutated HaloTag[22-297](M2F) control. A no transfection control (NTC) is shown that was measured identically except without introduction of an expression plasmid.

FIG. 154. Mutations improving performance of split HaloTag in a model protein:protein interaction system. HeLa cells transiently transfected with plasmids expressing different mutants of HaloTag[22-297](M2F) fragment fused to FRB-EGFP and HaloTag[3-19] fused to FKBP were incubated with 1 micromolar Rapamycin overnight at 37° C. at 24 hours post-transfection. The next day, the cells were labeled with 100 nM JF646 HaloTag ligand before detecting fluorescence activity at indicated time points. Non-transfected cell (NTC) control is included for reference.

FIG. 155. Fold response of mutations improving performance of split HaloTag in a model protein:protein interaction system. HeLa cells transiently transfected with plasmids expressing different mutants of HaloTag[22-297](M2F) fragment fused to FRB-EGFP and HaloTag[3-19] fused to FKBP were incubated with 1 micromolar Rapamycin overnight at 37° C. at 24 hours post-transfection. The next day, the cells were labeled with 100 nM JF646 HaloTag ligand before detecting fluorescence activity at indicated time points. The fold response of each assay condition was calculated as the ratio of fluorescence signal for +Rapamycin/−Rapamycin treated cells. Non-transfected cell (NTC) control is included for reference.

FIG. 156. Comparison of maximum fluorescence and fold response of mutations improving performance of split HaloTag in a model protein:protein interaction system. HeLa cells transiently transfected with plasmids expressing different mutants of HaloTag[22-297](M2F) fragment fused to FRB-EGFP and HaloTag[3-19] fused to FKBP were incubated with 1 micromolar Rapamycin overnight at 37° C. at 24 hours post-transfection. The next day, the cells were labeled with 100 nM JF646 HaloTag ligand before detecting fluorescence activity at indicated time points. The fold response of each assay condition was calculated as the ratio of fluorescence signal for +Rapamycin/−Rapamycin treated cells. Non-transfected cell (NTC) control is included for reference. This plot shows the comparison of fold responses vs. the total fluorescent activity of each mutant.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a polypeptide” is a reference to one or more polypeptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-fluorescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., fluorescent intensity of an active fluorophore).

As used herein, when referring to amino acid sequences or positions within an amino acid sequence, the phrase “corresponding to” refers to the relative position of an amino acid residue or an amino acid segment with the sequence being referred to, not necessarily the specific identity of the amino acids at that position. For example, a “peptide corresponding to positions 36 through 48 of SEQ ID NO: 1” may comprise less than 100% sequence identity with positions 36 through 48 of SEQ ID NO: 1 (e.g., >70% sequence identity), but within the context of the composition or system being described the peptide relates to those positions.

As used herein, the term “system” refers to multiple components (e.g., devices, compositions, etc.) that find use for a particular purpose. For example, two separate biological molecules, whether present in the same composition or not, may comprise a system if they are useful together for a shared purpose.

As used herein, the term “complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance (facilitation) to form a complex (e.g., from interaction elements), for example, to place the elements in the proper conformation for complementarity, to place the elements in the proper proximity for complementarity, to co-localize complementary elements, to lower interaction energy for complementary, to overcome insufficient affinity for one another, etc.

As used herein, the term “complex” refers to an assemblage or aggregate of molecules (e.g., peptides, polypeptides, etc.) in direct and/or indirect contact with one another. In one aspect, “contact,” or more particularly “direct contact,” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., peptides, polypeptides, etc.) is formed under assay conditions such that the complex is thermodynamically favored (e.g., compared to a non-aggregated, or non-complexed, state of its component molecules). As used herein the term “complex,” unless described as otherwise, refers to the assemblage of two or more molecules (e.g., peptides, polypeptides, etc.).

As used herein, the term “interaction element” refers to a moiety that assists or facilitates the bringing together of two or more structural elements (e.g., peptides, polypeptides, etc.) to form a complex. In some embodiments, a pair of interaction elements (a.k.a. “interaction pair”) is attached to a pair of structural elements (e.g., peptides, polypeptides, etc.), and the attractive interaction between the two interaction elements facilitate formation of a complex of the structural elements. Interaction elements may facilitate formation of a complex by any suitable mechanism (e.g., bringing structural elements into proximity, placing structural elements in proper conformation for stable interaction, reducing activation energy for complex formation, combinations thereof, etc.). An interaction element may be a protein, polypeptide, peptide, small molecule, cofactor, nucleic acid, lipid, carbohydrate, antibody, etc. An interaction pair may be made of two of the same interaction elements (i.e., homopair) or two different interaction elements (i.e., heteropair). In the case of a heteropair, the interaction elements may be the same type of moiety (e.g., polypeptides) or may be two different types of moieties (e.g., polypeptide and small molecule). In some embodiments, in which complex formation by the interaction pair is studied, an interaction pair may be referred to as a “target pair” or a “pair of interest,” and the individual interaction elements are referred to as “target elements” (e.g., “target peptide,” “target polypeptide,” etc.) or “elements of interest” (e.g., “peptide of interest,” “polypeptide or interest,” etc.).

As used herein, the term “low affinity” describes an intermolecular interaction between two or more entities that is too weak to result in significant complex formation between the entities, except at concentrations substantially higher (e.g., 2-fold, 5-fold, 10-fold, 100-fold, 1000-fold, or more) than physiologic or assay conditions, or with facilitation from the formation of a second complex of attached elements (e.g., interaction elements).

As used herein, the term “high affinity” describes an intermolecular interaction between two or more (e.g., three) entities that is of sufficient strength to produce detectable complex formation under physiologic or assay conditions, without facilitation from the formation of a second complex of attached elements (e.g., interaction elements).

As used herein, the term “preexisting protein” refers to an amino acid sequence that was in physical existence prior to a certain event or date. A “peptide that is not a fragment of a preexisting protein” is a short amino acid chain that is not a fragment or sub-sequence of a protein (e.g., synthetic or naturally-occurring) that was in physical existence prior to the design and/or synthesis of the peptide.

As used herein, the term “fragment” refers to a peptide or polypeptide that results from dissection or “fragmentation” of a larger whole entity (e.g., protein, polypeptide, enzyme, etc.), or a peptide or polypeptide prepared to have the same sequence as such. Therefore, a fragment is a subsequence of the whole entity (e.g., protein, polypeptide, enzyme, etc.) from which it is made and/or designed. A peptide or polypeptide that is not a subsequence of a preexisting whole protein is not a fragment (e.g., not a fragment of a preexisting protein). A peptide or polypeptide that is “not a fragment of a preexisting protein” is an amino acid chain that is not a subsequence of a protein (e.g., natural or synthetic) that was in physical existence prior to design and/or synthesis of the peptide or polypeptide. A fragment of a hydrolase or dehalogenase, as used herein, is a sequence which is less than the full-length sequence, but which alone cannot form a substrate binding site, and/or has substantially reduced or no substrate binding activity but which, in close proximity to a second fragment of a hydrolase or dehalogenase, exhibits substantially increased substrate binding activity. In one embodiment, a fragment of a hydrolase or dehalogenase is at least 5, e.g., at least 10, at least 20, at least 30, at least 40, or at least 50, contiguous residues of a wild-type hydrolase or a mutated hydrolase, or a sequence with at least 70% sequence identity thereto, and may not necessarily include the N-terminal or C-terminal residue or N-terminal or C-terminal sequences of the corresponding full length protein.

As used herein, the term “subsequence” refers to peptide or polypeptide that has 100% sequence identify with a portion of another, larger peptide, or polypeptide. The subsequence is a perfect sequence match for a portion of the larger amino acid chain.

The term “amino acid” refers to natural amino acids, unnatural amino acids, and amino acid analogs, all in their D and L stereoisomers, unless otherwise indicated, if their structures allow such stereoisomeric forms.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyrrolysine may also be considered proteinogenic amino acids

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to an amino acid (e.g., natural or unnatural, proteinogenic or non-proteinogenic) where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide, and S-(carboxymethyl)-cysteine sulfone.

As used herein, unless otherwise specified, the terms “peptide” and “polypeptide” refer to polymer compounds of two or more amino acids joined through the main chain by peptide amide bonds (—C(O)NH—). The term “peptide” typically refers to short amino acid polymers (e.g., chains having fewer than 30 amino acids), whereas the term “polypeptide” typically refers to longer amino acid polymers (e.g., chains having more than 30 amino acids).

As used herein, the terms “artificial” or “synthetic” refer to compositions and systems that are not naturally occurring. For example, an artificial or synthetic peptide, peptoid, or nucleic acid is one comprising a non-natural sequence (e.g., a peptide without 100% identity with a naturally-occurring protein or a fragment thereof).

As used herein in reference to the production of peptides and polypeptides, the term “synthesis” and linguistic variants thereof may refer to chemical peptide synthesis techniques as well as genetic expression of the peptides and polypeptides.

As used herein, a “conservative” amino acid substitution refers to the substitution of an amino acid in a peptide or polypeptide with another amino acid having similar chemical properties such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Amino acid residues may be divided into classes based on common side chain properties, for example: polar positive (or basic) (e.g., histidine (H), lysine (K), and arginine (R)); polar negative (or acidic) (e.g., aspartic acid (D), glutamic acid (E)); polar neutral (e.g., serine (S), threonine (T), asparagine (N), glutamine (Q)); non-polar aliphatic (e.g., alanine (A), valine (V), leucine (L), isoleucine (I), methionine (M)); non-polar aromatic (e.g., phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptide/polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the term “physiological conditions” encompasses any conditions compatible with living cells, e.g., predominantly aqueous conditions of a temperature, pH, salinity, chemical makeup, etc. that are compatible with living cells.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not however to be construed as limiting the sample types applicable to the present invention.

As used herein, the terms “fusion,” “fusion polypeptide,” and “fusion protein” refer to a chimeric protein containing a first protein or polypeptide of interest joined to a second different peptide, polypeptide, or protein (e.g., interaction element).

As used herein, the terms “conjugated” and “conjugation” refer to the covalent attachment of two molecular entities (e.g., post-synthesis and/or during synthetic production). The attachment of a peptide or small molecule tag to a protein or small molecule, chemically (e.g., “chemically” conjugated) or enzymatically, is an example of conjugation.

As used herein, the terms “polypeptide component” or “peptide component” are used synonymously with the terms “polypeptide component of a [mutant dehalogenase] complex” or “peptide component of a [mutant dehalogenase] complex.” Typically, as used herein, a polypeptide component or peptide component is capable of forming a complex with a second component to form a desired complex, under appropriate conditions.

As used herein, the term “dehalogenase” refers to an enzyme that catalyzes the removal of a halogen atom from a substrate. The term “haloalkane dehalogenase” refers to an enzyme that catalyzes the removal of a halogen from a haloalkane substrate to produce an alcohol and a halide. Dehalogenases and haloalkyl dehalogenases belong to the hydrolase enzyme family, and may be referred to herein or elsewhere as such.

As used herein, the term “modified dehalogenase” refers to a dehalogenase variant (artificial variant) that has mutations that prevent the release of the substrate from the protein following removal of the halogen, resulting in a covalent bond between the substrate and the modified dehalogenase. Because the modified dehalogenase does not release the substrate, it is not capable of turnover, and is not a classical enzyme. The HALOTAG system (Promega) is a commercially available modified dehalogenase and substrate system.

As used herein, the term “circularly-permuted” (“cp”) refers to a polypeptide in which the N- and C-termini have been joined together, either directly or through a linker, to produce a circularly-permuted polypeptide, and then the circularly-permuted polypeptide is opened at a location other than between the N- and C-termini to produce a new linear polypeptide with termini different from the termini in the original polypeptide. The location at which the circularly-permuted polypeptide is opened is referred to herein as the “cp site.” Circular permutants include those polypeptides with sequences and structures that are equivalent to a polypeptide that has been circularized and then opened. Thus, a cp polypeptide may be synthesized de novo as a linear molecule and never go through a circularization and opening step. The preparation of circularly permutated derivatives is described in WO95/27732; incorporated by reference in its entirety.

As used herein, the term “split” (“sp”) refers to refers to a polypeptide that has been divided into two fragments at an interior site of the original polypeptide. The fragments of a sp polypeptide may reconstitute the activity of the original polypeptide if they are structurally complementary and able to form an active complex. A nomenclature herein for referring to split components of a polypeptide recites a position number from the full polypeptide that corresponds to the last residue in the N-terminal component of the split polypeptide. For example, if a polypeptide is 100 residues in length, a sp52 version of that polypeptide comprises a first fragment corresponding to positions 1-52 of the parent polypeptide and a second fragment corresponding to positions 53-100 of the parent polypeptide. As another example, spHT(45) refers to a split variant of the commercially-available HALOTAG protein in which the first fragment comprises residues 1-45 of the HALOTAG polypeptide sequence and the second fragment comprises residues 46-297 of the HALOTAG polypeptide sequence. Alternatively, a component of a split polypeptide may be expressed herein by referring to the name of the polypeptide from which it is derived, the residues within the source polypeptide that are present in the component (in brackets), followed by any substitutions in the component relative to the source polypeptide (in parenthesis). For example, a split component of the commercially-available HALOTAG protein corresponding to position 22-297 of the HALOTAG sequence could be written HaloTag[22-297]. If the second position of the component contained a M to F substitution, the components could be referred to as HaloTag[22-297](M2F). Components may contain an N-terminal methionine residues not present in the source sequence; such residues are counted in referring to the location of substitutions but not in the numbering of the fragment within the source polypeptide.

As used herein, the term “gapped” refers to split variant of a polypeptide that is missing a segment of the original polypeptide. For example, a “gapped sp polypeptide” is one that is missing a segment of the original sequence that occurs at the site of the split.

As used herein, the term “overlapped” refers to split variant of a polypeptide that contains a duplication of a segment of the original polypeptide. For example, an “overlapped sp polypeptide” is one in which a segment of the original sequence adjacent to the split site is present (duplicated) at the C-terminus of a first fragment and the N-terminus of the second fragment.

### DETAILED DESCRIPTION

Provided herein are peptide and polypeptide sequences that structurally assemble to form active, modified dehalogenase structures capable of binding (e.g., covalently) to a haloalkyl ligand. In particular, provided herein are split dehalogenase variants that assemble through structural complementation into active dehalogenase complexes, and systems and methods of use thereof.

Split mutant proteins, i.e., enzymes mutated to inhibit or eliminate catalytic activity, find use in revealing and analyzing protein interaction within cells, e.g., where each portion (fragment) of the split protein is fused to a different protein. Provided herein are split mutated hydrolases, such as those derived from the commercially available HALOTAG protein (Promega) and/or mutated hydrolases disclosed in U.S. published application 20060024808, the disclosure of which is incorporated by reference herein.

Even though these mutant hydrolases are not technically enzymes (no substrate turnover), the stable binding of a substrate thereto is dependent on proper protein structure. The consequence of re-associating the split fragments of a mutated hydrolase differs from that of a split enzyme system because the labeling function of a mutated hydrolase is retained on one of the fragments even after it has separated from its partner, whereas split enzymes are only active while they are brought together and bear no artifact of their prior activity after they are separated. In effect, the labeling reaction of a split mutant hydrolase provides a molecular memory of a protein interaction. In the case of fluorogenic ligands, the label is retained on one of the fragments, but may not be detectable after complex dissociation (since the fluorogen-activating contacts with the protein may be disrupted/absent); therefore, the combination of split dehalogenase and fluorogenic ligands produce a unique situation of permanent labeling, but with dynamic (on/off) fluorescence detection of the retained label.

As an example of a mutated hydrolase, a mutated dehalogenase provides for efficient labeling within a living cell or lysate thereof. This labeling is only conditional on the presence or expression of the protein and the presence of the labeled hydrolase substrate. In contrast, the labeling of a split mutant dehalogenase is dependent on a specific protein interaction occurring within the cell and the presence of the labeled hydrolase substrate. For instance, beta-arrestin may be fused with one fragment of a mutated hydrolase, and a G-coupled receptor may be fused with the other fragment. Upon receptor stimulation in the presence of the labeled substrate, beta-arrestin binds to the receptor causing a labeling reaction of either the receptor fusion or the beta-arrestin fusion (depending on which portion of the mutated hydrolase contains the reactive nucleophilic amino acid).

In some embodiments, provided herein is a split mutant hydrolase (e.g., split modified dehalogenase) system, which includes a first fragment of a hydrolase fused to a protein of interest and a second fragment of the hydrolase optionally fused to a ligand of the first protein of interest. At least one of the hydrolase fragments has a substitution that if present in a full-length mutant hydrolase (e.g., modified dehalogenase) having the sequence of the two fragments, forms a bond with a hydrolase substrate that is more stable than the bond formed between the corresponding full length wild type hydrolase and the hydrolase substrate. In one embodiment, each fragment of the hydrolase is fused to a protein of interest and the proteins of interest interact, e.g., bind to each other. In another embodiment, one hydrolase fragment is fused to a protein of interest, which interacts with a molecule in a sample. In another embodiment, in the presence of an agent (one or more agents of interest), or under certain conditions, a complex is formed by the binding of a fusion having the protein of interest fused to a first hydrolase fragment, to a second protein fused to a second hydrolase fragment or to the second hydrolase fragment and a cellular molecule.

Thus, the two fragments of the hydrolase (e.g., modified dehalogenase) together provide a mutant hydrolase that is structurally related to (and comprises significant sequence identity/similarity to (e.g., >70%)) a full-length hydrolase, but includes at least one amino acid substitution that results in covalent binding of the hydrolase substrate. The full-length mutant hydrolase lacks or has reduced catalytic activity relative to the corresponding full length wild type hydrolase, and specifically binds substrates which may be specifically bound by the corresponding full length wild-type hydrolase, however, no product or substantially less product, e.g., 2-, 10-, 100-, or 1000-fold less, is formed from the interaction between the mutant hydrolase and the substrate under conditions, which result in product formation by a reaction between the corresponding full length wild type hydrolase and substrate. The lack of, or reduced amounts of, product formation by the mutant hydrolase is due to at least one substitution in the full-length mutant hydrolase, which substitution results in the mutant hydrolase forming a bond with the substrate, which is more stable than the bond formed between the corresponding full length wild-type hydrolase and the substrate.

HALOTAG is a 297-residue self-labeling polypeptide (33 kDa) derived from a bacterial hydrolase (dehalogenase) enzyme, which has modified to covalently bind to its ligand, a haloalkane moiety. The HALOTAG ligand can be linked to solid surfaces (e.g., beads) or functional groups (e.g., fluorophores), and the HALOTAG polypeptide can be fused to various proteins of interest, allowing covalent attachment of the protein of interest to the solid surface or functional group.

The HALOTAG polypeptide is a hydrolase (e.g., modified dehalogenase) with a genetically modified active site, which specifically binds to the haloalkane ligand chloroalkane linker with an enhanced and increased rate of ligand binding (Pries et al. The Journal of Biological Chemistry. 270(18):10405-11; incorporated by reference in its entirety). The reaction that forms the bond between the protein tag and chloroalkane linker is fast and essentially irreversible under physiological conditions (Waugh DS (June 2005). Trends in Biotechnology. 23(6):316-20; incorporated by reference in its entirety). In the natural hydrolase enzyme, nucleophilic attack of the chloroalkane reactive linker causes displacement of the halogen with an amino acid residue, which results in the formation of a covalent alkyl-enzyme intermediate. This intermediate would then be hydrolyzed by an amino acid residue within the wild-type hydrolase (Chen et al. (February 2005) Current Opinion in Biotechnology. 16(1):35-40; incorporated by reference in its entirety). This would lead to regeneration of the enzyme following the reaction. However, with HALOTAG, the modified haloalkane dehalogenase, the reaction intermediate cannot proceed through the second reaction because it cannot be hydrolyzed due to the mutation in the enzyme. This causes the intermediate to persist as a stable covalent adduct with which there is no associated back reaction (Marks et al. (August 2006) Nature Methods. 3 (8): 591-6; incorporated by reference in its entirety).

HALOTAG fusion proteins can be expressed using standard recombinant protein expression techniques (Adams et al. (May 2002) Journal of the American Chemical Society. 124(21):6063-76; incorporated by reference in its entirety). Since the HALOTAG polypeptide is a relatively small protein, and the reactions are foreign to mammalian cells, there is no interference by endogenous mammalian metabolic reactions (Naested et al. The Plant Journal. 18(5):571-6; incorporated by reference in its entirety). Once the fusion protein has been expressed, there is a wide range of potential areas of experimentation including enzymatic assays, cellular imaging, protein arrays, determination of sub-cellular localization, and many additional possibilities (Janssen DB (April 2004). Current Opinion in Chemical Biology. 8(2):150-9; incorporated by reference in its entirety).

Various HALOTAG ligands, functional groups, fusions, assays, modifications, uses, etc. are described in U.S. Pat. Nos. 8,748,148; 9,593,316; 10,246,690; 8,742,086; 9,873,866; 10,604,745; U.S. Pat. App. 2009/0253131; U.S. Pat. App. 2010/0273186; 20130337539; U.S. Pat. App. 2012/0258470; U.S. Pat. App. 2012/0252048; U.S. Pat. App. 2011/0201024; U.S. 2014/0322794; each of which is incorporated by reference in their entireties.

Since reversible protein complementation systems and biosensors have been demonstrated to be particularly useful tools for measuring functional dynamics with cell imaging, such as protein interactions or changes in metabolite concentration, experiments were conducted during development of embodiments herein to identify regions within the HALOTAG sequence that are amenable to design strategies that allow control of its self-labeling activity in a dynamic way. A comprehensive screen was first performed to identify all possible circular permutation sites in the HALOTAG protein that retain activity and stability in the context of a single polypeptide and/or conditionally-separable fragments. Using the information gained from this screen, split HALOTAG pairs were designed and tested.

In some embodiments, provided herein are HALOTAG-based systems tailored for functional biology, such as split HATOTAG polypeptides, with properties similar to existing full-length protein in terms of stability, solubility, and expression of the fragments, with the additional characteristic of being able to reconstitute a significant fraction of its activity upon reconstitution of the full enzyme. HALOTAG ligands of particular importance to certain embodiments herein include fluorogenic ligands. Systems combining spHT can be engineered to have a range of fragment affinities to enable both facilitated and spontaneous complementation systems. Split HALOTAG systems facilitate endogenous tagging of proteins and make fluorogenic ligands or sensors better through higher signal, stability, dynamic range, etc. The HALOTAG-based functional biology tools described herein are well suited for measuring protein dynamics in live cells using fluorescence imaging, an application where other technologies lack the utility of HALOTAG's self-labeling activity or sensitivity of fluorescent chloroalkane ligands.

As described herein, embodiments are not limited to the HALOTAG sequence. In some embodiments, provided herein are split modified dehalogenases that differ in sequence from SEQ ID NO: 1. In some embodiments, provided herein are split dehalogenases that lack the mutation(s) (e.g., 272 and/or 106) that produce covalent bonding to the haloalkane substrate. Such sp dehalogenases are true enzymes capable of substrate turnover, but otherwise comprising the sequences and characteristics of the embodiments described herein.

Experiments were conducted during development of embodiments herein to examine split dehalogenases, their ability to assemble into active dehalogenase structures, and their ability to activate fluorogenic substrates. Initially, a comprehensive screen of all circular permutants of HaloTag (cpHT) revealed that 228/296 (77%) reacted with CA-TMR, and 50 variants had at least 10% of native HT activity on CA-AlexaFluor488. Seventeen cpHT variants had increased thermal stability relative to HT, and 38 variants exhibited activity recovery after thermal denaturation, presumably by protein refolding. The most active variants by Alexa Fluor488 velocity clustered in a region distal from the lid domain (residues 133-215), but this effect may be particular to this substrate, which is negatively-charged and may be sensitive to lid domain perturbations. Indeed, when using the neutral TMR ligand, the clustering effect was less apparent. With the exception of cpHTs near residue 111 and 120, all the refolding variants were localized to the lid domain, and all the thermostabilized variants were also in the lid domain. From these results, 22 candidates identified in the cpHT screen were pursued for further testing as true split proteins (spHT). A set of spHT variants, as fusions to FRB and FKBP, were identified which exhibit rapamycin-inducible complementation, evidenced by activation of a fluorogenic HT ligand (e.g., spHT(133), spHT(145), spHT(157), spHT(180), and spHT(195), etc.). This functionality extends to pairs of spHT fragments containing varying degrees of sequence overlap localized to the lid subdomain of HT. Further investigation into disturbances in the lid subdomain revealed the critical function of Helix 8 in activating bound fluorogenic ligands. The spHT complexes displayed diverse behaviors in terms of reversibility, with three fully-reversible complexes and one irreversible complex identified in rapamycin/FK506 competition experiments, and an overall stabilizing effect noted for the JF646-bound states of all the complexes. spHT-FRB/FKBP fragments were co-expressed in mammalian cells and noted that the complexes form spontaneously, presumably through co-translational folding. Taken together, this work demonstrates a wide functional utility for spHT designs, several of which display unique properties.

In some embodiments, provided herein are spHT polypeptides and systems thereof. In particular sp-modified dehalogenases are provided that are capable of reconstituting all or a portion of the activity of the parent dehalogenase.

In some embodiments, the polypeptide, peptides, fragments, and combinations thereof described herein are derived from a modified dehalogenase sequence of SEQ ID NO: 1:

MAEIGTGFPFDPHYVEVLGERMHYVDVGPRDGTPVLFLHGNPTSSYVWR
NIIPHVAPTHRCIAPDLIGMGKSDKPDLGYFFDDHVRFMDAFIEALGLE
EVVLVIHDWGSALGFHWAKRNPERVKGIAFMEFIRPIPTWDEWPEFARE
TFQAFRITDVGRKLIIDQNVFIEGTLPMGVVRPLTEVEMDHYREPFLNP
VDREPLWRFPNELPIAGEPANIVALVEEYMDWLHQSPVPKLLFWGTPGV
LIPPAEAARLAKSLPNCKAVDIGPGLNLLQEDNPDLIGSEIARWLSTLE
ISG.

In some embodiments, peptides and polypeptides herein comprise at least 70% sequence identity with all or a portion of SEQ ID NO: 1 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, peptides and polypeptides herein comprise 100% sequence identity with all or a portion of SEQ ID NO: 1. In some embodiments, peptides and polypeptides herein comprise at least 70% sequence similarity with all or a portion of SEQ ID NO: 1 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, peptides and polypeptides herein comprise 100% sequence similarity with all or a portion of SEQ ID NO: 1.

In some embodiments, peptides or polypeptides herein comprise an A at a position corresponding to position 2 of SEQ ID NO: 1. In other embodiments, peptides or polypeptides herein comprise an S at a position corresponding to position 2 of SEQ ID NO: 1. In some embodiments, peptides or polypeptides herein comprise a V at a position corresponding to position 47 of SEQ ID NO: 1. In some embodiments, peptides or polypeptides herein comprise a T at a position corresponding to position 58 of SEQ ID NO: 1. In some embodiments, peptides or polypeptides herein comprise a G at a position corresponding to position 78 of SEQ ID NO: 1. In some embodiments, peptides or polypeptides herein comprise a F at a position corresponding to position 88 of SEQ ID NO: 1. In some embodiments, peptides or polypeptides herein comprise a M at a position corresponding to position 89 of SEQ ID NO: 1. In some embodiments, peptides or polypeptides herein comprise a F at a position corresponding to position 128 of SEQ ID NO: 1. In some embodiments, peptides or polypeptides herein comprise a T at a position corresponding to position 155 of SEQ ID NO: 1. In some embodiments, peptides or polypeptides herein comprise a K at a position corresponding to position 160 of SEQ ID NO: 1. In some embodiments, peptides or polypeptides herein comprise a V at a position corresponding to position 167 of SEQ ID NO: 1. In some embodiments, peptides or polypeptides herein comprise a T at a position corresponding to position 172 of SEQ ID NO: 1. In some embodiments, peptides or polypeptides herein comprise a M at a position corresponding to position 175 of SEQ ID NO: 1. In some embodiments, peptides or polypeptides herein comprise a G at a position corresponding to position 176 of SEQ ID NO: 1. In some embodiments, peptides or polypeptides herein comprise a N at a position corresponding to position 195 of SEQ ID NO: 1. In some embodiments, peptides or polypeptides herein comprise a E at a position corresponding to position 224 of SEQ ID NO: 1. In some embodiments, peptides or polypeptides herein comprise a D at a position corresponding to position 227 of SEQ ID NO: 1. In some embodiments, peptides or polypeptides herein comprise a K at a position corresponding to position 257 of SEQ ID NO: 1. In some embodiments, peptides or polypeptides herein comprise an A at a position corresponding to position 264 of SEQ ID NO: 1. In some embodiments, peptides or polypeptides herein comprise a N at a position corresponding to position 272 of SEQ ID NO: 1. In some embodiments, peptides or polypeptides herein comprise a L at a position corresponding to position 273 of SEQ ID NO: 1. In some embodiments, peptides or polypeptides herein comprise a S at a position corresponding to position 291 of SEQ ID NO: 1. In some embodiments, peptides or polypeptides herein comprise a T at a position corresponding to position 292 of SEQ ID NO: 1. In some embodiments, peptides or polypeptides herein comprise a E at a position corresponding to position 294 of SEQ ID NO: 1. In some embodiments, peptides or polypeptides herein comprise a I at a position corresponding to position 295 of SEQ ID NO: 1. In some embodiments, peptides or polypeptides herein comprise a S at a position corresponding to position 296 of SEQ ID NO: 1. In some embodiments, peptides or polypeptides herein comprise a G at a position corresponding to position 297 of SEQ ID NO: 1.

In some embodiments, peptides or polypeptides herein do not have an S at a position corresponding to position 2 of SEQ ID NO: 1. In some embodiments, peptides or polypeptides herein do not have a L at a position corresponding to position 47 of SEQ ID NO: 1. In some embodiments, peptides or polypeptides herein do not have a S at a position corresponding to position 58 of SEQ ID NO: 1. In some embodiments, peptides or polypeptides herein do not have a D at a position corresponding to position 78 of SEQ ID NO: 1. In some embodiments, peptides or polypeptides herein do not have a Y at a position corresponding to position 88 of SEQ ID NO: 1. In some embodiments, peptides or polypeptides herein do not have a L at a position corresponding to position 89 of SEQ ID NO: 1. In some embodiments, peptides or polypeptides herein do not have a C at a position corresponding to position 128 of SEQ ID NO: 1. In some embodiments, peptides or polypeptides herein do not have an A at a position corresponding to position 155 of SEQ ID NO: 1. In some embodiments, peptides or polypeptides herein do not have a E at a position corresponding to position 160 of SEQ ID NO: 1. In some embodiments, peptides or polypeptides herein do not have an A at a position corresponding to position 167 of SEQ ID NO: 1. In some embodiments, peptides or polypeptides herein do not have an A at a position corresponding to position 172 of SEQ ID NO: 1. In some embodiments, peptides or polypeptides herein do not have a K at a position corresponding to position 175 of SEQ ID NO: 1. In some embodiments, peptides or polypeptides herein do not have a C at a position corresponding to position 176 of SEQ ID NO: 1. In some embodiments, peptides or polypeptides herein do not have a K at a position corresponding to position 195 of SEQ ID NO: 1. In some embodiments, peptides or polypeptides herein do not have an A at a position corresponding to position 224 of SEQ ID NO: 1. In some embodiments, peptides or polypeptides herein do not have a N at a position corresponding to position 227 of SEQ ID NO: 1. In some embodiments, peptides or polypeptides herein do not have a E at a position corresponding to position 257 of SEQ ID NO: 1. In some embodiments, peptides or polypeptides herein do not have a T at a position corresponding to position 264 of SEQ ID NO: 1. In some embodiments, peptides or polypeptides herein do not have a H at a position corresponding to position 272 of SEQ ID NO: 1. In some embodiments, peptides or polypeptides herein do not have a Y at a position corresponding to position 273 of SEQ ID NO: 1. In some embodiments, peptides or polypeptides herein do not have a P at a position corresponding to position 291 of SEQ ID NO: 1. In some embodiments, peptides or polypeptides herein do not have an A at a position corresponding to position 292 of SEQ ID NO: 1. In some embodiments, peptides or polypeptides herein do not have an amino acid at a position corresponding to position 294 of SEQ ID NO: 1. In some embodiments, peptides or polypeptides herein do not have an amino acid at a position corresponding to position 295 of SEQ ID NO: 1. In some embodiments, peptides or polypeptides herein do not have an amino acid at a position corresponding to position 296 of SEQ ID NO: 1. In some embodiments, peptides or polypeptides herein do not have an amino acid at a position corresponding to position 297 of SEQ ID NO: 1.

As described herein, embodiments are not limited to the HALOTAG sequence. In some embodiments, provided herein are split modified dehalogenases that differ in sequence from SEQ ID NO: 1. In some embodiments, provided herein are split dehalogenases that lack the mutation(s) (e.g., 272 and/or 106) that produce covalent bonding to the haloalkane substrate. Such split dehalogenases are true enzymes capable of substrate turnover, but otherwise comprising the sequences and characteristics of the embodiments described herein.

In some embodiments, a sp dehalogenase (e.g., spHT) comprises two peptide and/or polypeptide components that collectively comprise at least 70% sequence identity with all or a portion of SEQ ID NO: 1 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). For example, the first peptide/polypeptide component of the sp polypeptide corresponds to a first portion of SEQ ID NO: 1 (e.g., at least 70% sequence identity to the first portion) and the first peptide/polypeptide component of the sp polypeptide corresponds to a second portion of SEQ ID NO: 1 (e.g., at least 70% sequence identity to the second portion). In some embodiments, a sp dehalogenase (e.g., spHT) comprises two fragments that collectively comprise 100% sequence identity with all or a portion of SEQ ID NO: 1. For example, the first fragment of the sp polypeptide has 100% sequence identity to a first portion of SEQ ID NO: 1 and the second fragment of the sp polypeptide has 100% sequence identity to a second portion SEQ ID NO: 1.

In some embodiments, a sp dehalogenase (e.g., spHT) comprises two peptide and/or polypeptide components that collectively comprise at least 70% sequence similarity with all or a portion of SEQ ID NO: 1 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). For example, the first peptide/polypeptide component of the sp polypeptide corresponds to a first portion of SEQ ID NO: 1 (e.g., at least 70% sequence similarity to the first portion), and the first peptide/polypeptide component of the sp polypeptide corresponds to a second portion of SEQ ID NO: 1 (e.g., at least 70% sequence similarity to the second portion). In some embodiments, a sp dehalogenase (e.g., spHT) comprises two fragments that collectively comprise 100% sequence similarity with all or a portion of SEQ ID NO: 1. For example, the first fragment of the sp polypeptide has 100% sequence similarity to a first portion of SEQ ID NO: 1, and the second fragment of the sp polypeptide has 100% sequence similarity to a second portion SEQ ID NO: 1.

In some embodiments, a sp dehalogenase (e.g., spHT) comprises a sp site. The sp site is an internal location in the parent sequence that defines the C-terminus of the first component or fragment and the N-terminus of the second component or fragment of the sp dehalogenase. For example, if a theoretical a 100 amino acid polypeptide were split with a sp site between residues 57 and 58 of the parent polypeptide (referred to herein as a sp site of 57), the first component polypeptide would correspond to positions 1-57 of SEQ ID NO: 1, and the second component polypeptide would correspond to positions 58-100 of SEQ ID NO: 1. In some embodiments herein, a sp site within SEQ ID NO: 1 may occur at any position from position 5 of SEQ ID NO:1 to position 290 of SEQ ID NO: 1. In some embodiments, SEQ ID NOS: 2-577 are exemplary components of spHT polypeptides having 100% sequence identity to SEQ ID NO: 1. In some embodiments, an active spHT complex is formed between two fragments that collectively comprise amino acids corresponding to each position in SEQ ID NO: 1. For example, a polypeptide having a sequence of SEQ ID NO: 26 and a peptide having a sequence of SEQ ID NO: 27 collectively comprise amino acids corresponding to each position in SEQ ID NO: 1. Any pairs of peptide and polypeptides (or two polypeptides) corresponding to two of SEQ ID NO:S 2-577 and together comprising amino acids corresponding to each position in SEQ ID NO: 1 (without deletion or duplication of positions) find use in embodiments herein. In some embodiments, a spHT dehalogenase comprises any of the following pairs of fragment: SEQ ID NOS: 2 and 3, 4 and 5, 6 and 7, 8 and 9, 10 and 11, 12 and 13, 14 and 15, 16 and 17, 18 and 19, 20 and 21, 22 and 23, 24 and 25, 26 and 27, 28 and 29, 30 and 31, 32 and 33, 34 and 35, 36 and 37, 38 and 39, 40 and 41, 42 and 43, 44 and 45, 46 and 47, 48 and 49, 50 and 51, 52 and 53, 54 and 55, 56 and 57, 58 and 59, 60 and 61, 62 and 63, 64 and 65, 66 and 67, 68 and 69, 70 and 71, 72 and 73, 74 and 75, 76 and 77, 78 and 79, 80 and 81, 82 and 83, 84 and 85, 86 and 87, 88 and 89, 90 and 91, 92 and 93, 94 and 95, 96 and 97, 98 and 99, 100 and 101, 102 and 103, 104 and 105, 106 and 107, 108 and 109, 110 and 111, 112 and 113, 114 and 115, 116 and 117, 118 and 119, 120 and 121, 121, 122 and 123, 124 and 125, 126 and 127, 128 and 129, 130 and 131, 132 and 133, 134 and 135, 136 and 137, 138 and 139, 140 and 141, 142 and 143, 144 and 145, 146 and 147, 148 and 149, 150 and 151, 152 and 153, 154 and 155, 156 and 157, 158 and 159, 160 and 161, 172 and 173, 174 and 175, 176 and 177, 178 and 179, 180 and 181, 182 and 183, 184 and 185, 186 and 187, 188 and 189, 190 and 191, 192 and 193, 194 and 195, 196 and 197, 198 and 199, 200 and 201, 202 and 203, 204 and 205, 206 and 207, 208 and 209, 190 and 211, 212 and 213, 214 and 215, 216 and 217, 218 and 219, 220 and 221, 222 and 223, 224 and 225, 226 and 227, 228 and 229, 300 and 301, 302 and 303, 304 and 305, 306 and 307, 308 and 309,310 and 311,312 and 313,314 and 315,316 and 317,318 and 319,320 and 321,322 and 323, 324 and 325, 326 and 327, 328 and 329, 330 and 331, 332 and 333, 334 and 335, 336 and 337, 338 and 339, 340 and 341, 342 and 343, 344 and 345, 346 and 347, 348 and 349, 350 and 351, 352 and 353, 354 and 355, 356 and 357, 358 and 359, 360 and 361, 362 and 363, 364 and 365, 366 and 367, 368 and 369, 370 and 371, 372 and 373, 374 and 375, 376 and 377, 378 and 379, 380 and 381, 382 and 383, 384 and 385, 386 and 387, 388 and 389, 390 and 391, 392 and 393, 394 and 395, 396 and 397, 398 and 399, 400 and 401, 402 and 403, 404 and 405, 406 and 407, 408 and 409, 410 and 411, 412 and 413, 414 and 415, 416 and 417, 418 and 419, 420 and 421, 422 and 423, 424 and 425, 426 and 427, 428 and 429, 430 and 431, 432 and 433, 434 and 435, 436 and 437, 438 and 439, 440 and 441, 442 and 443, 444 and 445, 446 and 447, 448 and 449, 450 and 451, 452 and 453, 454 and 455, 456 and 457, 458 and 459, 460 and 461, 462 and 463, 464 and 465, 466 and 467, 468 and 469, 470 and 471, 472 and 473, 474 and 475, 476 and 477, 478 and 479, 480 and 481, 482 and 483, 484 and 485, 486 and 487, 488 and 489, 490 and 491, 492 and 493, 494 and 495, 496 and 497, 498 and 499, 500 and 501, 502 and 503, 504 and 505, 506 and 507, 508 and 509, 510 and 511, 512 and 513, 514 and 515, 516 and 517, 518 and 519, 520 and 521, 522 and 523, 524 and 525, 526 and 527, 528 and 529, 530 and 531, 532 and 533, 534 and 535, 536 and 537, 538 and 539, 540 and 541, 542 and 543, 544 and 545, 546 and 547, 548 and 549, 550 and 551, 552 and 553, 554 and 555, 556 and 557, 558 and 559, 560 and 561, 562 and 563, 564 and 565, 566 and 567, 568 and 569, 570 and 571, 572 and 573, 574 and 575, and 576 and 577.

In some embodiments, a spHT comprises a peptide and polypeptide (or two polypeptides) pair corresponding to two of SEQ ID NOS: 2-577 together comprising amino acids corresponding to each position in SEQ ID NO: 1, but with a deletion of up to 40 amino acids in length (e.g., 1, 2, 3, 4, 5, 6, 7, 8, 9, 10, 15, 20, 25, 30, 35, 40, or ranges therebetween) at the C-terminus or N-terminus of one or both of fragments. For example, a pair corresponding to SEQ ID NOS: 7 and 28 together correspond to positions of SEQ ID NO: 1, but with an 11 residue deletion. In some embodiments, any pairs of SEQ ID NOS: 2-577, together corresponding to the sequence of SEQ ID NO: 1, but with deletions of up to 40 amino acids, are within the scope of spHTs herein. In some embodiments, the deletion is adjacent to the split site. In some embodiments, the deletion corresponds to the N- or C-terminus of SEQ ID NO: 1.

In some embodiments, a spHT comprises a peptide and polypeptide (or two polypeptides) pair corresponding to two of SEQ ID NOS: 2-577 together comprising amino acids corresponding to each position in SEQ ID NO: 1, but with a duplication of up to 40 amino acids in length (e.g., 1, 2, 3, 4, 5, 6, 7, 8, 9, 10, 15, 20, 25, 30, 35, 40, or ranges therebetween) at the C-terminus or N-terminus of one or both of fragments. For example, a pair corresponding to SEQ ID NOS: 6 and 29 together correspond to positions of SEQ ID NO: 1, but with an 11 residue duplication. In some embodiments, any pairs of SEQ ID NOS: 2-577, together corresponding to the sequence of SEQ ID NO: 1, but with duplications of up to 40 amino acids, are within the scope of spHTs herein. In some embodiments, the duplication is adjacent to the split site. In some embodiments, the duplication corresponds to the N- or C-terminus of SEQ ID NO: 1.

Fragments utilizing any sp sites, for example, corresponding to a position between position 5 and position 290 of SEQ ID NO: 1 are readily envisioned and within the scope herein.

In some embodiments, spHTs are provided with a sp site corresponding to position 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 313, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 310, 311, 312, 313, 314, 315, 316, 317, 318, 319, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290 of SEQ ID NO: 1.

In some embodiments, spHTs are provided with a sp site corresponding to a position between positions 5 and 13, 36 and 51, 63 and 72, 84 and 92, 104 and 130, 142 and 148, 160 and 174, 186 and 189, 311 and 313, 221 and 229, or 269 and 290, of SEQ ID NO: 1.

In some embodiments, sp peptides and polypeptides are provided having 70%-100% sequence identity to one of SEQ ID NOS: 2-557 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, sp peptides and polypeptides are provided having 70%-100% sequence similarity to one of SEQ ID NOS: 2-557 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity).

In some embodiments, pairs of sp peptides and/or polypeptides are provided that are capable of forming active sp dehalogenase complexes (active spHT complexes). Such pairs comprise at least 70% sequence identity or similarity to two of SEQ ID NOS: 2-557, and together comprise residues corresponding to 100% of the positions in SEQ ID NO: 1, allowing for up to 40 deletions or duplications at the C- or N-terminus of the peptides/polypeptides.

In some embodiments, the first fragment of a spHT complementary pair corresponds to position 1 through position 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 313, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 310, 311, 312, 313, 314, 315, 316, 317, 318, 319, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290 of SEQ ID NO: 1.

In some embodiments, the second fragment of a spHT complementary pair corresponds to position 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 313, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 310, 311, 312, 313, 314, 315, 316, 317, 318, 319, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290 of SEQ ID NO: 1 through position 294 of SEQ ID NO: 1.

In some embodiments, the duplicated portion of a spHT complementary pair is 1-40 amino acids in length (e.g., 1, 2, 3, 4, 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, or ranges therebetween).

In some embodiments, the deleted portion of a spHTs complementary pair is 1-40 amino acids in length (e.g., 1, 2, 3, 4, 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, or ranges therebetween).

The exemplary spHT fragment sequences of SEQ ID NOS: 2-577 comprise 100% sequence identity to portions of SEQ ID NO: 1; there are no portions of these sequences that do not align with 100% sequence identity to SEQ ID NO: 1. However, as described herein, spHT peptides and polypeptides may have less than 100% sequence identity with SEQ ID NO: 1 (e.g., >70%, >75%, >80%, >85%, >90%, >95%, >96%, >97%, >98%, >99%, but less than 100% sequence identity). Therefore, peptides and polypeptide having less than 100% sequence identity with one of SEQ ID NOS: 2-577 (e.g., >70%, >75%, >80%, >85%, >90%, >95%, >96%, >97%, >98%, >99%, but less than 100% sequence identity) are provided herein and find use in the complementary pairs and complexes herein.

In some embodiments, a spHT complementary pair herein comprises a peptide corresponding to SEQ ID NO: 578 and a polypeptide corresponding to SEQ ID NO: 1188. SEQ NOS: 578 and 1188 are fragments of SEQ ID NO: 1 and have 100% sequence identity to portions of SEQ ID NO: 1. In some embodiments, a spHT complementary pair comprises a peptide having 100% sequence identity to SEQ ID NO: 578; such a peptide is referred to herein as “SmHT.” In some embodiments, a spHT complementary pair comprises a polypeptide having 100% sequence identity to SEQ ID NO: 1188; such a polypeptide is referred to herein as “LgHT.” Extensive experiments were conducted during development of embodiments herein to analyze variants of SmHT and LgHT. SEQ ID NOS: 579-1187 correspond to peptide variants having at least one and up to all positions of SEQ ID NO: 588 substituted. A peptide of each of SEQ ID NOS: 578-1187 was synthesized and tested for various characteristics, including the ability to form an active complex with a complementary LgHT variant polypeptide. SEQ ID NOS: 1189-3033 correspond to polypeptide variants having one or more substitutions relative to SEQ ID NO: 1188. A polypeptide of each of SEQ ID NOS: 1188-3033 was synthesized and tested for various characteristics, including the ability to form an active complex with a complementary SmHT variant peptide.

In some embodiments, provided herein is a SmHT peptide or SmHT variant peptide having at least 70% (e.g., 70%, 75%, 80%, 85%, 90%, 95%, 100%, or ranges therebetween) sequence similarity (e.g., conservative or semi-conservative similarity) with one of SEQ ID NOS: 578-1187. In some embodiments, a peptide corresponds to SmHT (SEQ ID NO: 578), but with one or more (e.g., 2, 3, 4, 5, 6, 7, 8, 9, 10, 11, 12, 13, 14, 15, 16, 17, or ranges therebetween) of the substitutions of one or more of SEQ ID NOS: 588-1187 relative to SEQ ID NO: 578. In some embodiments, a SmHT variant has 1-8 (e.g., 1, 2, 3, 4, 5, 6, 7, 8, or ranges therebetween) non-conservative substitutions relative to one of SEQ ID NOS: 578-1187.

In some embodiments, provided herein is a SmHT peptide or SmHT variant peptide comprising:

(SEQ ID NO: 3034)
X₁X₂X₃X₄X₅(F/W/Y/M/H)X₇(F/W/Y/D/R)X₉X₁₀X₁₁(F/W/Y/M/H/
R)(V/I/L/M/A/C)X₁₄(V/I/L/A/C/MI/L/F/W)X₁₆X₁₇;
and/or
(SEQ ID NO: 3035)
X₁X₂X₃X₄X₅(F/W/Y)X₇(F/W/Y)X₉X₁₀X₁₁(F/W/Y)(V/I/L/M)X₁₄
(V/I/L)X₁₆X₁₇;

wherein each X is any amino acid (e.g., proteinogenic amino acid).

In some embodiments, provided herein is a LgHT polypeptide or LgHT variant polypeptide having at least 70% (e.g., 70%, 75%, 80%, 85%, 90%, 95%, 100%, or ranges therebetween) sequence similarity (e.g., conservative or semi-conservative similarity) with one of SEQ ID NOS: 1188-3033. In some embodiments, a polypeptide corresponds to LgHT (SEQ ID NO: 1188), but with one or more (e.g., 2, 3, 4, 5, 6, 7, 8, 9, 10, 11, 12, 13, 14, 15, 16, 17, 18, 19, 20, 25, 30, or more, or ranges therebetween) of the substitutions of one or more of SEQ ID NOS: 1189-3033 relative to SEQ ID NO: 1188. In some embodiments, a LgHT variant has at least 70% (e.g., 70%, 75%, 80%, 85%, 90%, 95%, 100%, or ranges therebetween) sequence identity with one of SEQ ID NOS: 1188-3033.

In some embodiments, provided herein is a spHT complementary pair comprising (a) a SmHT peptide or SmHT variant peptide having (1) at least 70% (e.g., 70%, 75%, 80%, 85%, 90%, 95%, 100%, or ranges therebetween) sequence similarity (e.g., conservative or semi-conservative similarity) with one of SEQ ID NOS: 578-1187, (2) one or more (e.g., 2, 3, 4, 5, 6, 7, 8, 9, 10, 11, 12, 13, 14, 15, 16, 17, or ranges therebetween) substitutions relative to SEQ ID NO: 578, and/or (3) 1-8 (e.g., 1, 2, 3, 4, 5, 6, 7, 8, or ranges therebetween) non-conservative substitutions relative to one of SEQ ID NOS: 578-1187; and (b) a LgHT polypeptide or LgHT variant polypeptide having (1) at least 70% (e.g., 70%, 75%, 80%, 85%, 90%, 95%, 100%, or ranges therebetween) sequence similarity (e.g., conservative or semi-conservative similarity) with one of SEQ ID NOS: 1188-3033, (2) one or more (e.g., 2, 3, 4, 5, 6, 7, 8, 9, 10, 11, 12, 13, 14, 15, 16, 17, 18, 19, 20, 25, 30, or more, or ranges therebetween) substitutions relative to SEQ ID NO: 1188, and/or (3) at least 70% (e.g., 70%, 75%, 80%, 85%, 90%, 95%, 100%, or ranges therebetween) sequence identity with one of SEQ ID NOS: 1188-3033.

In some embodiments, the split hydrolase (e.g., spHT) and fragments thereof have enhanced thermal stability relative to the parent hydrolase sequence (e.g., HALOTAG).

The formation of a spHT complex from two complementary fragments may be reversible or irreversible. In some embodiments, a spHT complex is capable of being denatured, renatured, and having its activity reconstituted. In some embodiments, such spHTs find use in methods that comprise exposing samples containing the spHTs to denaturing conditions (e.g., manufacturing conditions, storage conditions, etc.) prior to substrate binding.

In some embodiments, provided herein are a fusions of the split hydrolases (e.g., dehalogenases (e.g., HALOTAG, etc.), etc.) with proteins of interest, interaction elements, localization elements, heterologous sequences, peptide tags, luciferases, or bioluminescent complexes, etc.

In certain embodiments, both fragments of a split hydrolase (e.g., spHT) are fused to heterologous sequences. In some embodiments, the heterologous sequences are substantially the same and specifically bind to each other, e.g., form a dimer, optionally in the absence of one or more exogenous agents. In another embodiment, the heterologous sequences are different and specifically bind to each other, optionally in the absence of one or more exogenous agents. In one embodiment, one hydrolase fragment is fused to a heterologous sequence and that heterologous sequence interacts with a cellular molecule. In another embodiment, each hydrolase fragment is fused to a heterologous sequence and in the presence of one or more exogenous agents or under specified conditions, the heterologous sequences interact. For instance, in the presence of rapamycin, a fragment of a hydrolase fused to rapamycin binding protein (FRB) and another fragment fused to FK506 binding protein (FKBP), yields a complex of the two fusion proteins. In one embodiment, in the presence of the exogenous agent(s) or under different conditions, the complex of fusion proteins does not form. In one embodiment, one heterologous sequence includes a domain, e.g., 3 or more amino acid residues, which optionally may be covalently modified, e.g., phosphorylated, that noncovalently interacts with a domain in the other heterologous sequence. The two fragments of the hydrolase, at least one of which is fused to a protein of interest, may be employed to detect reversible interactions, e.g., binding of two or more molecules, or other conformational changes or changes in conditions, such as pH, temperature or solvent hydrophobicity, or irreversible interactions.

The rapamycin/FRB/FKBP system provides an example of a small molecule inducing a protein-protein interaction that can be detected/monitored by the spHT systems herein. However, other systems of inducing formation of a spHT complex are within the scope herein. Other small molecule induced protein interactions find use in embodiments herein. Additionally, proteins interact (i.e., associate or dissociate) as a result of other events in cells that impact their local concentrations, e.g., direct physical association, co-localization, additive/subtractive abundance caused by stabilizing or degrading stimulus, additive/subtractive abundance controlled at genetic level (i.e., up-regulation, down-regulation). Embodiments herein find use in monitoring such effects in vitro and in vivo.

Heterologous sequences useful in the invention include, but are not limited to, those which interact in vitro and/or in vivo. For instance, the fusion protein may comprise (1) hydrolase fragment (e.g., portion of a spHT) and (2) an enzyme of interest, e.g., luciferase, RNasin or RNase, and/or a channel protein, a receptor, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, a transcription factor, a transporter protein and/or a targeting sequence, e.g., a myristilation sequence, a mitochondrial localization sequence, or a nuclear localization sequence, that directs the hydrolase fragment, for example, a fusion protein, to a particular location. The protein of interest, which is fused to the hydrolase fragment, may be a fragment of a wild-type protein, e.g., a functional or structural domain of a protein, such as a domain of a kinase, a transcription factor, and the like. The protein of interest may be fused to the N-terminus or the C-terminus of the fragment (e.g., portion of a spHT). In one embodiment, the fusion protein comprises a protein of interest at the N-terminus, and another protein, e.g., a different protein, at the C-terminus, of the fragment (e.g., portion of a spHT). For example, the protein of interest may be an antibody. Optionally, the proteins in the fusion are separated by a linker, e.g., a linker sequence of 1-20 amino acids (e.g., 1, 2, 3, 4, 5, 6, 7, 8, 9, 10, 11, 12, 13, 14, 15, 16, 17, 18, 19, and 20 acid residues). In some embodiments, the presence of a linker in a fusion protein of the invention does not substantially alter the function of either protein in the fusion relative to the function of each individual protein. For any particular combination of proteins in a fusion, a wide variety of linkers may be employed. In one embodiment, the linker is a sequence recognized by an enzyme, e.g., a cleavable sequence, or is a photocleavable sequence.

Exemplary heterologous sequences include but are not limited to sequences such as those in FRB and FKBP, the regulatory subunit of protein kinase (PKa-R) and the catalytic subunit of protein kinase (PKa-C), a src homology region (SH2) and a sequence capable of being phosphorylated, e.g., a tyrosine containing sequence, an isoform of 14-3-3, e.g., 14-3-3t (see Mils et al., 3100), and a sequence capable of being phosphorylated, a protein having a WW region (a sequence in a protein which binds proline rich molecules (see Ilsley et al., 3102; and Einbond et al., 1996), and a heterologous sequence capable of being phosphorylated, e.g., a serine and/or a threonine containing sequence, as well as sequences in dihydrofolate reductase (DHFR) and gyrase B (GyrB).

As described throughout, the spHT peptides and polypeptides provided herein find use as portions of fusion proteins with peptides, polypeptides, antibodies, antibody fragments, and proteins of interest. For instance, the invention provides a fusion protein comprising (1) a spHT peptide or polypeptide and (2) amino acid sequences for a protein or peptide of interest, e.g., sequences for a marker protein, e.g., a selectable marker protein, an enzyme of interest, e.g., luciferase, RNasin, RNase, and/or GFP, a nucleic acid binding protein, an extracellular matrix protein, a secreted protein, an antibody or a portion thereof such as Fc, a bioluminescence protein, a receptor ligand, a regulatory protein, a serum protein, an immunogenic protein, a fluorescent protein, a protein with reactive cysteines, a receptor protein, e.g., NMDA receptor, a channel protein, e.g., an ion channel protein such as a sodium-, potassium- or a calcium-sensitive channel protein including a HERG channel protein, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, e.g., a protease substrate, a transcription factor, a protein destabilization sequence, or a transporter protein, e.g., EAAT1-4 glutamate transporter, as well as targeting signals, e.g., a plastid targeting signal, such as a mitochondrial localization sequence, a nuclear localization signal or a myristilation sequence, that directs the fusion to a particular location.

In some embodiments, a fusion protein includes (1) spHT peptide or polypeptide and (2) a protein that is associated with a membrane or a portion thereof, e.g., targeting proteins such as those for endoplasmic reticulum targeting, cell membrane bound proteins, e.g., an integrin protein or a domain thereof such as the cytoplasmic, transmembrane and/or extracellular stalk domain of an integrin protein, and/or a protein that links the mutant hydrolase to the cell surface, e.g., a glycosylphosphoinositol signal sequence.

Fusion partners may include those having an enzymatic activity. For example, a functional protein sequence may encode a kinase catalytic domain (Hanks and Hunter, 1995), producing a fusion protein that can enzymatically add phosphate moieties to particular amino acids, or may encode a Src Homology 2 (SH2) domain (Sadowski et al., 1986; Mayer and Baltimore, 1993), producing a fusion protein that specifically binds to phosphorylated tyrosines.

In some embodiments, a fusion comprises an affinity domain, including peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include HisV5 (HHHHH) (SEQ ID NO:13), HisX6 (HHHHHH) (SEQ ID NO:3), C-myc (EQKLISEEDL) (SEQ ID NO:4), Flag (DYKDDDDK) (SEQ ID NO:5), SteptTag (WSHPQFEK) (SEQ ID NO:6), hemagluttinin, e.g., HA Tag (YPYDVPDYA) (SEQ ID NO:7), GST, thioredoxin, cellulose binding domain, RYIRS (SEQ ID NO:8), Phe-His-His-Thr (SEQ ID NO:9), chitin binding domain, S-peptide, T7 peptide, SH2 domain, C-end RNA tag, WEAAAREACCRECCARA (SEQ ID NO:10), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D_{9K}, calbindin D_{28K}, and calretinin, inteins, biotin, streptavidin, MyoD, Id, leucine zipper sequences, and maltose binding protein.

In some embodiments, a split hydrolase fragment described herein (e.g., spHT) is fused to a reporter protein. In some embodiments, the reporter is a bioluminescent reporter (e.g., expressed as a fusion protein with the spHT). In certain embodiments, the bioluminescent reporter is a luciferase. In some embodiments, a luciferase is selected from those found in Omphalotus olearius, fireflies (e.g., Photinini), Renilla reniformis, Aequoria, mutants thereof, portions thereof, variants thereof, and any other luciferase enzymes suitable for the systems and methods described herein. In some embodiments, the bioluminescent reporter is a modified, enhanced luciferase enzyme from Oplophorus (e.g., NANOLUC enzyme from Promega Corporation, SEQ ID NO: 3 or a sequence with at least 70% identity (e.g., >70%, >80%, >90%, >95%) thereto). Exemplary bioluminescent reporters are described, for example, in U.S. Pat. App. No. 2010/0281552 and U.S. Pat. App. No. 2012/0174242, both of which are herein incorporated by reference in their entireties.

In some embodiments, a split hydrolase fragment described herein (e.g., spHT) is fused to a peptide or polypeptide component of a commercially available NanoLuc®-based technologies (e.g., NanoLuc® luciferase, NanoBiT, NanoTrip, NanoBRET, etc.). PCT Appln. No. PCT/US2010/033449, U.S. Pat. No. 8,557,970, PCT Appln. No. PCT/2011/059018, and U.S. Pat. No. 8,669,103 (each of which is herein incorporated by reference in their entirety and for all purposes) describe compositions and methods comprising bioluminescent polypeptides that find use as heterologous sequences in the fusions herein. Such polypeptides find use in embodiments herein and can be used in conjunction with the compositions and methods described herein. PCT Appln. No. PCT/US14/26354 and U.S. Pat. No. 9,797,889 (each of which is herein incorporated by reference in their entirety and for all purposes) describe compositions and methods for the assembly of bioluminescent complexes; such complexes, and the peptide and polypeptide components thereof, find use as heterologous sequences in embodiments herein and can be used in conjunction with the compositions and methods described herein. In some embodiments, NanoBiT and other related technologies utilize a peptide component and a polypeptide component that, upon assembly into a complex, exhibit significantly-enhanced (e.g., 2-fold, 5-fold, 10-fold, 10²-fold, 10³-fold, 10⁴-fold, or more) luminescence in the presence of an appropriate substrate (e.g., coelenterazine or a coelenterazine analog) when compared to the peptide component and polypeptide component alone. In some embodiments, the NanoBiT peptides and polypeptides are fused to spHT fragments herein. U.S. Pat. Pub. 2020/0270586 and Intl. App. No. PCT/US19/36844 (herein incorporated by reference in their entireties and for all purposes) describe multipartite luciferase complexes (e.g., NanoTrip) that find use as heterologous sequences in embodiments herein and can be used in conjunction with the compositions and methods described herein.

In some embodiments, a sp dehalogenase finds use with a split reporter. In some embodiments, the fragments of a sp dehalogenase are tethered (e.g., fused, linked, etc.) to the fragments of a split reporter. Upon binding of the two entities, an active dehalogenase and an active reporter are formed. Examples of split fluorescent protein reporters include split GFP and split mCherry. In other embodiments, a first fragment of a split reporter (e.g., split fluorescent protein, split luciferase, etc.) is fused to a first fragment of a sp dehalogenase and a second fragment of the split reporter is linked to a haloalkane substrate. In such embodiments, upon formation of the active dehalogenase complex, the complex binds to the haloalkane substrate and the active reporter complex is assembled. In some embodiments, the fragments of a sp dehalogenase and/or a haloalkane are fused to other split proteins, such as split TEV protease or other enzymes.

We also envision our split HaloTag fragments being used in “dual tag” configurations, where split fragments of HaloTag are combined with split fragments of luciferases, fluorescent proteins, or other labeling/reporters (including SpyCatcher). For example, a HiBiT-spHaloTag fragment tag, or a GFP11-spHaloTag fragment tag. More broadly, there are split versions of other enzyme classes, such as split TEV protease, which could be created in these “dual tag” configurations as well.

As described herein, the spHT systems herein utilize haloalkane substrates. In some embodiments, the substrate is of formula (I): R-linker-A-X, wherein R is a solid surface, one or more functional groups, or absent, wherein the linker is a multiatom straight or branched chain including C, N, S, or O, or a group that comprises one or more rings, e.g., saturated or unsaturated rings, such as one or more aryl rings, heteroaryl rings, or any combination thereof, wherein A-X is a substrate for a dehalogenase, hydrolase, HALOTAG, or a spHT system herein (e.g., wherein A is (CH₂)₄₋₂₀ and X is a halide (e.g., Cl or Br)). Suitable substrates are described, for example, in U.S. Pat. Nos. 11,072,812; 11,028,424; 10,618,907; and 10,101,332; incorporated by reference in their entireties.

In some embodiments, R is one or more functional groups (such as a fluorophore, biotin, luminophore, or a fluorogenic or luminogenic molecule). Exemplary functional groups for use in the invention include, but are not limited to, an amino acid, protein, e.g., enzyme, antibody or other immunogenic protein, a radionuclide, a nucleic acid molecule, a drug, a lipid, biotin, avidin, streptavidin, a magnetic bead, a solid support, an electron opaque molecule, chromophore, MRI contrast agent, a dye, e.g., a xanthene dye, a calcium sensitive dye, e.g., 1-[2-amino-5-(2,7-dichloro-6-hydroxy-3-oxy-9-xanthenyl)-phenoxy]-2-(2′-am-ino-5′-methylphenoxy)ethane-N,N,N′,N′-tetraacetic acid (Fluo-3), a sodium sensitive dye, e.g., 1,3-benzenedicarboxylic acid, 4,4′-[1,4,10,13-tetraoxa-7,16-diazacyclooctadecane-7,16-diylbis(5-methoxy-6,2-benzofurandiyl)]bis (PBFI), a NO sensitive dye, e.g., 4-amino-5-methylamino-2′,7′-difluorescein, or other fluorophore. In one embodiment, the functional group is an immunogenic molecule, i.e., one which is bound by antibodies specific to that molecule.

In some embodiments, substrates of the invention are permeable to the plasma membranes of cells (i.e., capable of passing from the exterior of a cell (e.g., eukaryotic, prokaryotic) to the cellular interior without chemical, enzymatic, or mechanical disruption of the cell membrane).

In some embodiments, substrates herein comprise a cleavable linker, for example, those described in U.S. Pat. No. 10,618,907; incorporated by reference in its entirety.

In some embodiments, a substrate comprises a fluorescent functional group (R). Suitable fluorescent functional groups include, but are not limited to: stilbazolium derivatives (Marquesa et al. Mechanism-Based Strategy for Optimizing HaloTag Protein Labeling. ChemRxiv. Cambridge: Cambridge Open Engage; 2021; incorporated by reference in its entirety), xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLOUR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc.

In some embodiments, a substrate comprises a fluorogenic functional group (R). A fluorogenic functional group is one that produces and enhanced fluorescent signal upon binding of the substrate to a target (e.g., binding of a haloalkane to a modified dehalogenase). By producing significantly increased fluorescence (e.g., 10×, 31×, 50×, 100×, 310×, 500×, 100×, or more) upon target engagement, the problem of background signal is alleviated. Exemplary fluorogenic dyes for use in embodiments herein include the JANELIA FLUOR family of fluorophores, such as:

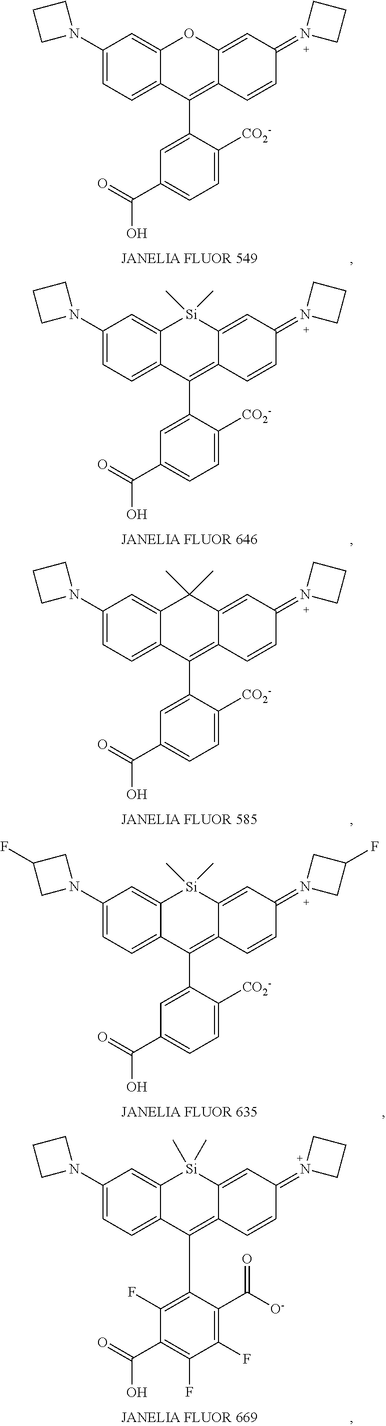

(see, e.g., U.S. Pat. Nos. 9,933,417; 10,018,624; 10,161,932; and 10,495,632; each of which is incorporated by reference in their entireties). In some embodiments, exemplary conjugates of JANELIA FLUOR 549 and JANELIA FLUOR 646 with haloalkane substrates for modified dehalogenase (e.g., HALOTAG) are commercially available (Promega Corp.). The use and design of fluorogenic functional groups, dyes, probes, and substrates is described in, for example, Grimm et al. Nat Methods. 3117 October; 14(10):987-994.; Wang et al. Nat Chem. 3120 February; 12(2):165-172; incorporated by reference in their entireties.

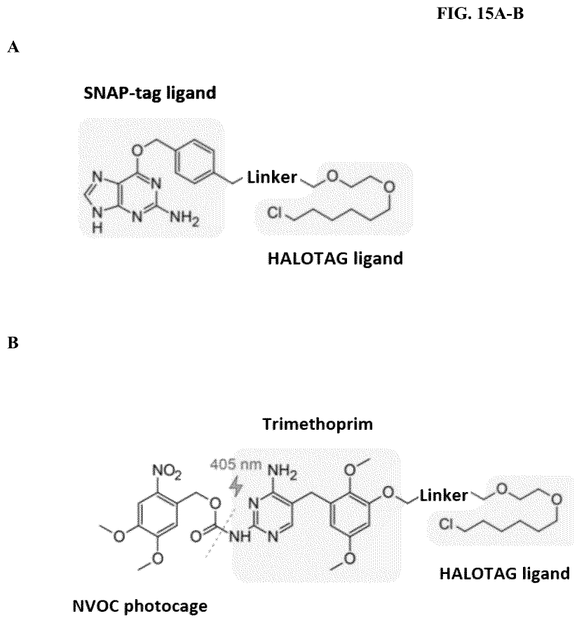

In some embodiments, ‘dual warhead’ substrates are provided that comprise a haloalkane moiety (e.g., a substrate for a modified dehalogenase (e.g., HALOTAG)) and a dimerization moiety that is a ligand (or capture element) for a second binding protein (capture element). For example, certain embodiments herein utilize a haloalkane linked to a SNAP-tag ligand (FIG. 15A; Cermakova & Hodges. Molecules 2018, 23(8), 1958; incorporated by reference in its entirety); a haloalkane linked to cTMP (FIG. 15B; Cermakova & Hodges. Molecules 2018, 23(8), 1958; incorporated by reference in its entirety)); a haloalkane linked to rapamycin-like moiety capable of binding to FKBP or FRB (Chen et al. ACS Chem. Biol. 2021, 16, 12, 2808-2815; incorporated by reference in its entirety); or other haloalkane ‘dual warhead’ ligands capable of binding to a modified dehalogenase (e.g., HALOTAG) and a second capture agent. In such embodiments, a system is provided comprising a split modified dehalogenase (spHT), a dual warhead substrate, and a capture agent capable of binding to the dimerization moiety (e.g., FKBP, FRB, SNAP-tag, eDHFR, etc.). In some embodiments, the capture agent and/or one or both fragments of the split modified dehalogenase (spHT) are provided as fusions with proteins of interest. In some embodiments, the dual warhead ligand triggers dimerization of (1) a split modified dehalogenase (spHT) and any elements bound or fused thereto with (2) the capture agent any elements bound or fused thereto. By adding another protein binding small molecule moiety onto a haloalkane, the dual warheads trigger close proximity of fusion partners to the split modified dehalogenase (spHT) and the capture agent. In some embodiments, a cell comprises two proteins of interest, one tagged by a fragment of a split modified dehalogenase (spHT) and the other tagged with a capture agent; in the presence of a dual warhead ligand comprising a haloalkane and a capture element for the capture agent, the tags dimerize and position the fused proteins of interest into close proximity. Such embodiments provide forced proximity of fusion partners to these proteins in the cell and control the timing and extent of their colocalization through the addition of the dual warhead ligand. Any suitable linkers may find use in assembly of dual warhead substrates. The linker may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ—) linkages, and the like. In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)). In some embodiments, the linker includes a combination of oligoethylene glycol linkages and carbamate linkages. In some embodiments, the linker has a formula —O(CH₂CH₂O)_{z1}—C(O)NH—(CH₂CH₂O)_{z2}—C(O)NH—(CH₂)_{z3}—(OCH₂CH₂)_{z4}O—, wherein z1, z2, z3, and z4 are each independently selected form 0, 1, 2, 3, 4, 5, and 6. For example, in some embodiments, the linker has a formula selected from:

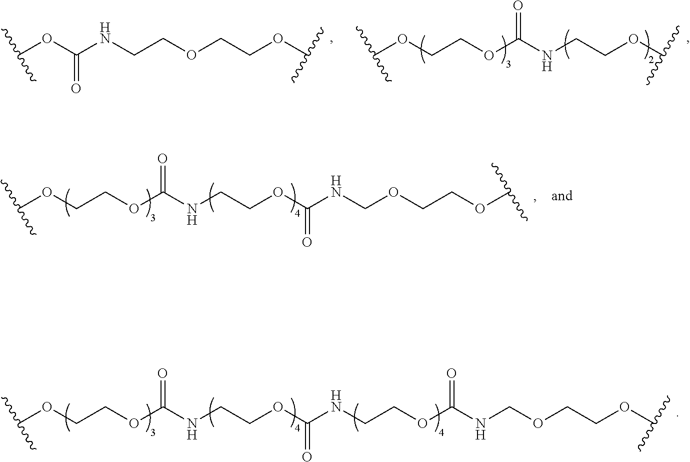

In some embodiments, a dual warhead that finds use in embodiments herein is a haloalkane linked to a ligand capable of engaging an E3 ubiquitin ligase (e.g., thalidomide, Cereblon E3 ubiquitin ligase, von Hippel-Lindau (VHL) E3 ligase or any other E3 ubiquitin ligase), otherwise known as a proteolysis targeting chimera (PROTAC). The haloalkane PROTAC is capable of binding to a modified dehalogenase or modified dehalogenase complex and an E3 ubiquitin ligase; recruitment of the E3 ligase results in ubiquitination and subsequent degradation via the proteasome of the to the modified dehalogenase (complex) and any protein components (e.g., a target protein) fused thereto. In some embodiments, the split dehalogenase systems herein find use in assays/systems to measure the kinetics of target protein ubiquitination or, in an endpoint format, for applications such as measuring compound dose-response curves. For example, in some embodiments, a target protein is expressed/provided in a sample as a fusion with a first component fragment of a split modified dehalogenase (e.g., spHT); the sample is contacted with a PROTAC of a haloalkane and a ligand capable of engaging an E3 ubiquitin ligase (e.g., thalidomide, Cereblon E3 ubiquitin ligase, von Hippel-Lindau (VHL) E3 ligase or any other E3 ubiquitin ligase); upon addition of a second component fragment of the split modified dehalogenase (e.g., spHT), the active modified dehalogenase complex is formed, the haloalkane is bound by the complex bringing the ligand in proximity of the target protein, resulting in ubiquitination and directing the fusion target to the proteasome for degradation.

In some embodiments, the components of the split dehalogenase have high affinity for one another, and therefore the split dehalogenase complex forms when the two components are in proximity to each other. The high affinity for the components of the split modified dehalogenase drives the formation of the split dehalogenase complex and the degradation of the target protein. In such embodiments, the second component could be added to the system at a specified time to induce degradation, could be localized to a specific location or compartment (e.g., cell type, organelle, tissue, etc.) where degradation will occur, or could conditionally expressed. In other embodiments, the components of the split dehalogenase have low affinity for one another, and a second interaction is required to induce the formation of the split dehalogenase complex. For example, the second component of the split dehalogenase is fused to a protein that binds the target protein or is tethered to a ligand for the target protein. Binding of this component to the target proteins allows formation of the split dehalogenase complex, which can in turn bind the haloalkane of the PROTAC and induce degradation.

In related embodiments, a target protein is expressed/provided in a sample as a fusion with (i) a first component fragment of a split modified dehalogenase (e.g., spHT) and (ii) a first interacting protein; the sample is contacted with a proteolysis targeting chimera (PROTAC) of a haloalkane and a ligand capable of engaging an E3 ubiquitin ligase (e.g., thalidomide); upon addition of a fusion of the second component fragment of the split modified dehalogenase (e.g., spHT) and a second interacting protein, the active modified dehalogenase complex is formed (facilitated by binding of the first and second interacting proteins), the haloalkane is bound by the complex bringing the ligase in proximity of the target protein, resulting in ubiquitination and directing the fusion target to the proteasome for degradation. In other embodiments, the complex formation and subsequent degradation is monitored by fluorescence, bioluminescence, and/or BRET. For example, in certain embodiments, a target protein is expressed/provided in a sample as a fusion with a luciferase (e.g., NANOLUC) or a component of a bioluminescent complex (e.g., a component of the NANOBIT system); a first component fragment of a split modified dehalogenase (e.g., spHT) is expressed/provided as a fusion with ubiquitin or an E3 ubiquitin ligase (e.g., thalidomide, Cereblon E3 ubiquitin ligase, von Hippel-Lindau (VHL) E3 ligase or any other E3 ubiquitin ligase); the sample is contacted with bifunctional ligand comprising a haloalkane and a molecule capable of binding to the target protein; upon addition of a second component fragment of the split modified dehalogenase (e.g., spHT) with high affinity of the first component fragment, the active modified dehalogenase complex is formed, the haloalkane is bound by the complex bringing the ubiquitin in proximity of the target protein, resulting in ubiquitination, directing the target to the proteasome for degradation, and extinguishing the signal from the luciferase. In similar embodiments, a component of the split modified dehalogenase is tethered to a fluorophore, such that BRET between the target fusion and the split modified dehalogenase can be used to monitor the system.

In other embodiments, a targeting chimera (TAC) system may utilize a haloalkane linked to a detectable moiety to monitor the system, rather than as a functional component of the system. For example, a first component of the modified dehalogenase is fused to ubiquitin, a second component of the modified dehalogenase (e.g., with low affinity for the first component) is fused to a target protein, and a haloalkane is linked to a fluorophore or other detectable moiety. Upon ubiquitin being brought into proximity of the target protein, the modified dehalogenase complex is forming, the haloalkane is bound, and the complex is labelled with the detectable moiety.

In some embodiments, split dehalogenase systems herein find use in various other targeting chimera (TAC) systems, such as: phosphorylation targeting chimera (PhosTAC; Chen et al. ACS Chem. Biol. 3121, 16, 12, 2808-2815; incorporated by reference in its entirety) systems, deubiquitinase targeting chimera (DUBTAC; Henning et al. Deubiquitinase-Targeting Chimeras for Targeted Protein Stabilization. bioRxiv; 2021. DOI: 10.1101/2021.04.30.441959; incorporated by reference in its entirety) systems, lysosome-targeting chimaera (LyTAC; Banik et al. Nature 584, 291-297 (2020); incorporated by reference in its entirety) systems, autophagy-targeting chimera (AUTAC; Takahashi et al. Mol Cell. 2019 Dec. 5; 76(5):797-810.e10; incorporated by reference in its entirety) systems, autophagy-tethering compound (ATTEC; Fu et al. Cell Research volume 31, pages 965-979 (2021); incorporated by reference in its entirety) systems, and oligo-based TACs. Dual warheads comprising a haloalkane and a ligand for any of the above TAC system may find use in embodiments herein. For example, PhosTACs are similar to the well-described PROTACs in their ability to induce ternary complexes, PhosTACs focus on recruiting a Ser/Thr phosphatase to a phosphosubstrate to mediate its dephosphorylation. PhosTACs extend the use of PROTAC technology beyond protein degradation via ubiquitination to also other protein post-translational modifications. For example, in some embodiments, a target protein is expressed/provided in a sample as a fusion with a first component fragment of a split modified dehalogenase (e.g., spHT); the sample is contacted with a phosphorylation targeting chimera (PhosTAC) of a haloalkane and a ligand capable of engaging an phosphatase enzyme; upon addition of a second component fragment of the split modified dehalogenase (e.g., spHT) with high affinity of the first component fragment, the active modified dehalogenase complex is formed, the haloalkane is bound by the complex bringing the ligand in proximity of the target protein, resulting in phosphorylation of the target protein.

In some embodiments, split dehalogenase systems herein find use is other targeting chimera systems in which a dual function ligand comprising a haloalkane and a ligand for a recruitable enzyme is used in combination with a fusion of a target protein and a fragment of a spHT to induce the enzymatic activity of the recruitable enzyme to the target protein upon introduction of the second high affinity spHT fragment to the system.

Systems and methods comprising any combinations of the above TAC systems/assays are within the scope herein.

In some embodiments, provided herein are isolated nucleic acid molecules (polynucleotides) comprising a nucleic acid sequence encoding a split hydrolase (e.g., spHT) fragments described herein. In some embodiments, such polynucleotides contain an open reading frame encoding a spHT or fragment thereof. In some embodiments, such polynucleotides are within an expression vector or integrated into the genomic material of a cell. In some embodiments, such polynucleotides further comprise regulatory elements such as a promotor. Further provided is an isolated nucleic acid molecule comprising a nucleic acid sequence encoding a fusion protein comprising a sp hydrolase fragment (e.g., spHT, etc.) and one or more amino acid residues at the N-terminus (a N-terminal fusion partner) and/or C-terminus (a C-terminal fusion partner). In one embodiment, the fusion protein comprises at least two different fusion partners (e.g., as described herein), one at the N-terminus and another at the C-terminus, where one of the fusions may be a sequence used for purification, e.g., a glutathione S-transferase (GST) or a polyHis sequence, a sequence intended to alter a property of the remainder of the fusion protein, e.g., a protein destabilization sequence, or a sequence which has a property which is distinguishable. In one embodiment, the isolated nucleic acid molecule comprises a nucleic acid sequence, which is optimized for expression in at least one selected host. Optimized sequences include sequences, which are codon optimized, i.e., codons that are employed more frequently in one organism relative to another organism, e.g., a distantly related organism, as well as modifications to add or modify Kozak sequences and/or introns, and/or to remove undesirable sequences, for instance, potential transcription factor binding sites. In one embodiment, the polynucleotide includes a nucleic acid sequence encoding a fragment of dehalogenase, which nucleic acid sequence is optimized for expression in a selected host cell. In one embodiment, the optimized polynucleotide no longer hybridizes to the corresponding non-optimized sequence, e.g., does not hybridize to the non-optimized sequence under medium or high stringency conditions. In another embodiment, the polynucleotide has less than 90%, e.g., less than 80%, nucleic acid sequence identity to the corresponding non-optimized sequence and optionally encodes a polypeptide having at least 80%, e.g., at least 85%, 90% or more, amino acid sequence identity with the polypeptide encoded by the non-optimized sequence.

Constructs, e.g., expression cassettes, and vectors comprising the isolated nucleic acid molecule, as well as host cells having one or more of the constructs, and kits comprising the isolated nucleic acid molecule, one or more constructs or vectors are also provided. Host cells include prokaryotic cells or eukaryotic cells such as a plant or vertebrate cells, e.g., mammalian cells, including but not limited to a human, non-human primate, canine, feline, bovine, equine, ovine or rodent (e.g., rabbit, rat, ferret, or mouse) cell. In some embodiments, the expression cassette comprises a promoter, e.g., a constitutive or regulatable promoter, operably linked to the nucleic acid molecule. In some embodiments, the expression cassette contains an inducible promoter. In certain embodiments, the invention includes a vector comprising a nucleic acid sequence encoding a fusion protein comprising a fragment of a dehalogenase. In some embodiments, optimized nucleic acid sequences, e.g., human codon optimized sequences, encoding at least a fragment of the hydrolase, and preferably the fusion protein comprising the fragment of a hydrolase, are employed in the nucleic acid molecules of the invention. The optimization of nucleic acid sequences is known to the art, see, for example WO 02/16944; incorporated by reference in its entirety.

Also provided are cells comprising the split hydrolase fragment(s) (e.g., spHT), polynucleotides, expression vector, etc. herein. In some embodiments, a component described herein is expressed within a cell. In some embodiments, a component herein is introduced to a cell, e.g., via transfection, electroporation, infection, cell fusion, or any other means.

In some embodiments, a system herein (e.g., comprising a sp hydrolase (e.g., spHT, etc.) may be employed to measure or detect various conditions and/or molecules of interest. For instance, protein-protein interactions are essential to virtually all aspects of cellular biology, ranging from gene transcription, protein translation, signal transduction and cell division and differentiation. Protein complementation assays (PCA) are one of several methods used to monitor protein-protein interactions. In PCA, protein-protein interactions bring two non-functional halves of an enzyme physically close to one another, which allows for re-folding into a functional enzyme. Interactions are therefore monitored by enzymatic activity. In protein complementation labeling (PCL), a covalent bond is created between the substrate and the complex resulting in cumulative labeling over time, thus increasing sensitivity for the detection of weak and/or rare protein-protein interactions. In a typical split enzyme system, if the complementation is disrupted, the signal generation is lost due to lack of or reduced substrate turnover. However, in a split labeling protein system (e.g., spHaloTag), the covalent nature of the label causes it to be retained on the split protein even after the complementation is disrupted. The demonstrated benefit of the latter is that for very low abundance, but regularly occurring molecular events (like neurotransmitters or hormones binding a receptor), a signal is accumulated over time (covalently) and eventually provides enough signal to detect the events—something that is difficult to do with a split enzyme system due to the rarity of the events leading to low turnover of the enzyme into signal.

In one embodiment, vectors encoding two complementing fragments of a mutant dehalogenase (e.g., spHT) at least one of which is fused to a protein of interest, or encoding two complementing fragments of a mutant dehalogenase each of which is fused to a protein of interest, are introduced to a cell, cell lysate, in vitro transcription/translation mixture, or supernatant, and a hydrolase substrate (e.g., haloalkane) labeled with a functional group is added thereto. Then the functional group is detected or determined, e.g., at one or more time points and relative to a control sample.

In some embodiments, provided herein are methods to detect an interaction between two proteins in a sample. The method includes providing a sample having a cell comprising a plurality of expression vectors of the invention, a lysate of the cell, or an in vitro transcription/translation reaction having the plurality of expression vectors of the invention, and a hydrolase substrate (e.g., haloalkane) with at least one functional group under conditions effective to allow for association of the first and second fusion proteins. The presence, amount, or location of the at least one functional group in the sample is detected.

In some embodiments, the invention provides a method to detect a molecule of interest in a sample. The method includes providing a sample having a cell having a plurality of expression vectors of the invention, a lysate thereof, an in vitro transcription/translation reaction having the plurality of expression vectors of the invention, and a hydrolase substrate (e.g., haloalkane) with at least one functional group under conditions effective to allow the first heterologous amino acid sequence to interact with a molecule of interest in the sample. The presence, amount, or location of the at least one functional group in the sample is detected, thereby detecting the presence, amount, or location of the molecule of interest.

Also provided herein are methods to detect an agent that alters the interaction of two proteins, which includes providing a sample having a cell comprising a plurality of expression vectors of the invention, a lysate thereof, or an in vitro transcription/translation reaction having a plurality of expression vectors of the invention, a hydrolase substrate (e.g., haloalkane) with at least one functional group, and an agent under conditions effective to allow for association of the first and second fusion proteins. The agent is suspected of altering the interaction of the first and second heterologous amino acid sequences. The presence or amount of the at least one functional group in the sample relative to a sample without the agent is detected.

In another embodiment, the invention provides a method to detect an agent that alters the interaction of a molecule of interest and a protein. The method includes providing a sample having a cell comprising a plurality of expression vectors of the invention, a lysate thereof, or an in vitro transcription/translation reaction having the plurality of expression vectors of the invention, a hydrolase substrate (e.g., haloalkane) with at least one functional group, and an agent suspected of altering the interaction between the heterologous amino acid sequence and a molecule of interest in the sample. The presence or amount of the functional group in the sample relative to a sample with the agent.

In some embodiments, provided herein are methods of detecting the presence of a molecule of interest. For instance, a cell is contacted with vectors comprising a promoter, e.g., a regulatable promoter, and a nucleic acid sequence encoding the two complementary fragments of a mutant hydrolase, at least one of which is fused to a protein which interacts with the molecule of interest. In one embodiment, a transfected cell is cultured under conditions in which the promoter induces transient expression of the fragments or regulated expression of one of the fragments and an activity associated with the labeled substrate is detected.

In some embodiments, a system herein (e.g., comprising a sp hydrolase (e.g., spHT, etc.) may be employed as a biosensor to detect the presence/amount of a molecule or interest or a particular condition (e.g., pH or temperature). Upon interacting with a molecule of interest or being subject to certain conditions, the biosensor undergoes a conformational change or is chemically altered which causes an alteration in activity. In some embodiments, a sp hydrolase herein comprises an interaction domain for a molecule of interest. For example, the biosensor could be generated to detect proteases (such as one to detect the presence of a particular viral protease, which in turn is indicator of the presence of the virus), kinases (for example, by inserting a kinase site into a reporter protein), RNAi (e.g., by inserting a sequence suspected of being recognized by RNAi into a coding sequence for a reporter protein, then monitoring reporter activity after addition of RNAi), a ligand, a binding protein such as an antibody, cyclic nucleotides such as cAMP or cGMP, or a metal such as calcium, by insertion of a suitable sensor region into the sp hydrolase (e.g., spHT, etc.). One or more sensor regions can be inserted at the C-terminus, the N-terminus, and/or at one or more suitable location in the sp hydrolase sequence, wherein the sensor region comprises one or more amino acids. One or all of the inserted sensor regions may include linker amino acids to couple the sensor to the remainder of the polypeptide. Examples of biosensors are disclosed in U.S. Pat. Appl. Publ. Nos. 2005/0153310 and 2009/0305280 and PCT Publ. No. WO 2007/120522 A2, each of which is incorporated by reference herein.

### EXPERIMENTAL

### Example 1

### Comprehensive Screen of Circular Dehalogenase Permutants

Plasmids encoding all possible circularly permuted versions of HaloTag, along with two linker control versions of non-permuted HaloTag with the linker simply appended the N- or C-terminus, were constructed by PCR, for a total of 298 gene constructs. The linker connecting the native N- and C-terminus was GSSGGGSSGGEPTTENLYFQ/SDNGSSGGGSSGG (TEV protease recognition sequence underlined, cleavable peptide bond indicated by slash). Expression was performed in E. coli, and cell lysates were prepared by addition of a chemical lysis reagent. Lysates were treated with TEV protease (or water as a negative control) and subjected to a panel of biochemical tests.

Lysates were assayed for protein solubility by centrifugation, followed by conjugation with 10 μM CA-TMR ligand and gel electrophoresis. To determine the thermal stability of each cpHT, lysates were heated to 40-90° C. for 30 min and cooled to room temperature, after which they were mixed with 10 nM CA-TMR and subject to fluorescence polarization (FP) measurements. Enzyme activity was measured quantitatively by mixing lysates with 10 nM CA-AlexaFluor488 and monitoring their FP change over 30 min.

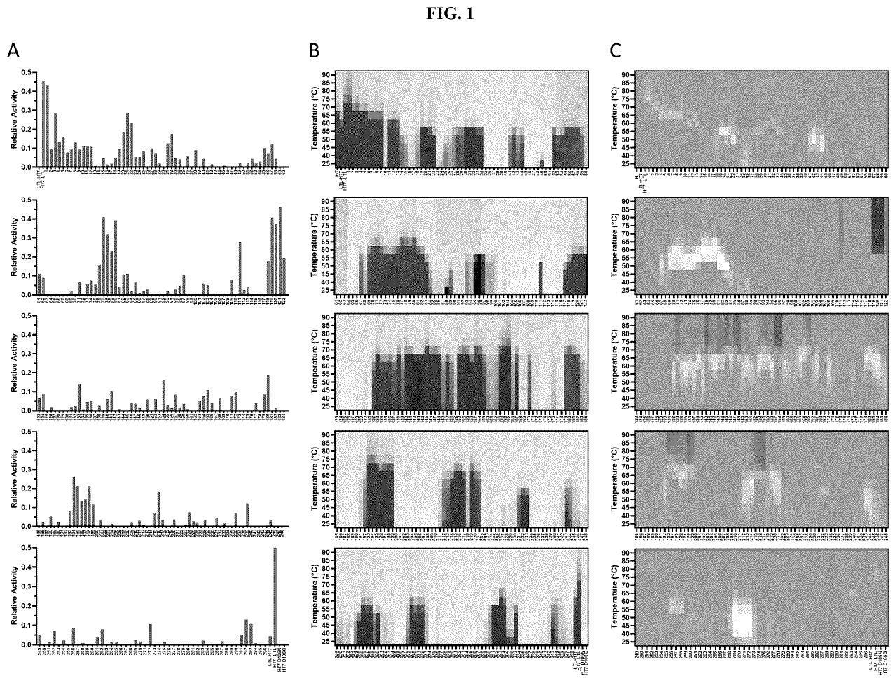

This screen revealed that 228/296 (77%) of cpHT variants reacted with CA-TMR, with the majority of these being soluble, and 50 variants had at least 10% of native HT activity on CA-AlexaFluor488 (FIG. 1). Seventeen cpHT variants had increased thermal stability relative to HT, and 38 variants exhibited activity recovery after thermal denaturation, presumably by protein refolding. The most active variants by Alexa Fluor488 velocity clustered in a region distal from the lid domain, but this effect may be particular to this substrate, which is negatively-charged and may be sensitive to lid domain perturbations. Indeed, when using the neutral TMR ligand in the solubility and stability assays, the clustering effect was less apparent. With the exception of cpHTs near residue 111 and 120, all the refolding variants were localized to the lid domain, and all the thermostabilized variants were also in the lid domain.

### Example 2

### Testing of Split Dehalogenase Variants

Selection of Candidates for spHT Screen from Comprehensive cpHT Screen

After completing the screen of all 298 possible circular permutants of HaloTag (cpHT) (See Example 1), 22 split sites were selected for testing as split HaloTag fragment pairs (spHT). Candidate spHT designs were selected based on characteristics of their cpHT counterparts, including thermal stability, expression, enzyme activity, and changes in biophysical properties upon cleavage of the TEV protease recognition sequence in the linker connecting the natural N- and C-termini. Particular interest was paid to variants which, upon TEV protease cleavage of the cpHT forms, exhibited the ability to renature, or refold, after thermal denaturation (e.g., circular permutants in the sequence region near residue 120).

Expression of spHT Fragments as Insoluble Fusions to Various Tags

An initial set of spHT N- and C-terminal fragments (spHT 80, 97, and 121) was expressed in E. coli as fusions to several different domains, including maltose-binding protein (MBP), a 6×-polyhistidine tag (His-tag), the large and small components of the bimolecular NanoBiT system (LgBiT and SmBiT). While moderate expression was noted for several of these fusions, all suffered from low solubility. The low solubility was attributed to the exposure of core hydrophobic residues, normally buried in the complete HT structure, which form aggregation-prone surfaces on the spHT fragments. Estimates based on NanoLuc activity place the solubility of these fragments at <5% in E. coli lysates.

Characterization of spHT Variants by Chemically-Induced Dimerization of FRB/FKBP Fusions

Despite low solubility, all 22 exemplary spHT designs were produced as fusions to FRB and FKBP domains. FRB and FKBP undergo chemically-induced, high-affinity heterodimerization in the presence of rapamycin; thus, spHT fragments fused to these domains can be brought into close proximity with one another by the addition of rapamycin, providing an assay for functional reconstitution of HaloTag enzyme activity. Each of the spHT fragments was fused (n=44) to FRB or FKBP, at either the N- or C-terminus, to generate a total of 176 unique fusion proteins and expressed in E. coli. Since the best orientation of FRB and FKBP relative to the spHT fragment domains cannot be predicted ab initio, all possible orientations and combinations were assayed (eight per spHT site). Fusion combinations were assayed using the fluorogenic JaneliaFluor 646 (JF646) ligand, in the presence of 50 nM rapamycin. JF646 was selected because it is available through the regular Promega catalog, has low background fluorescence (which enables direct fluorescence measurements in 96-well plates), and offers a higher stringency test than non-fluorogenic ligands (like TMR).

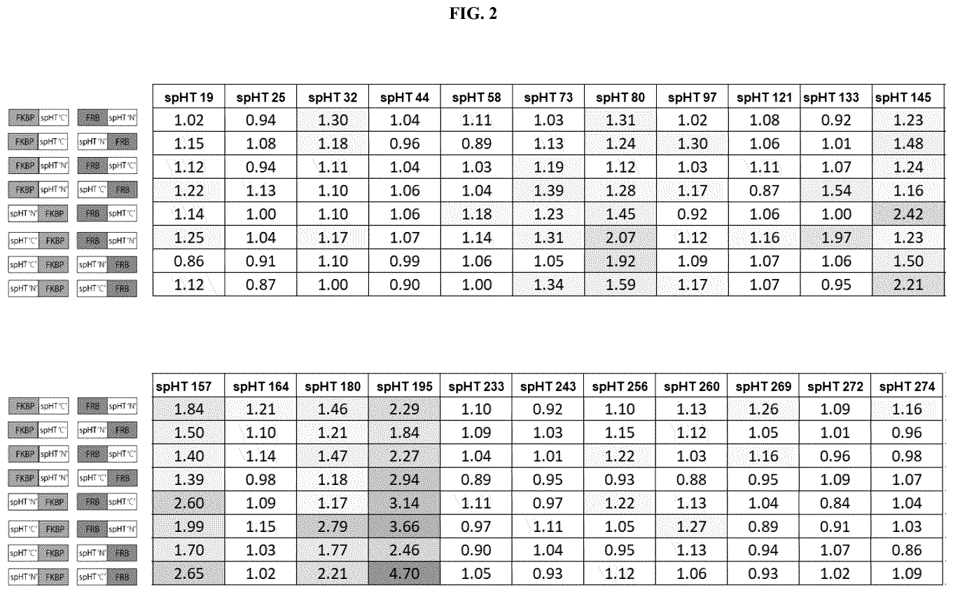

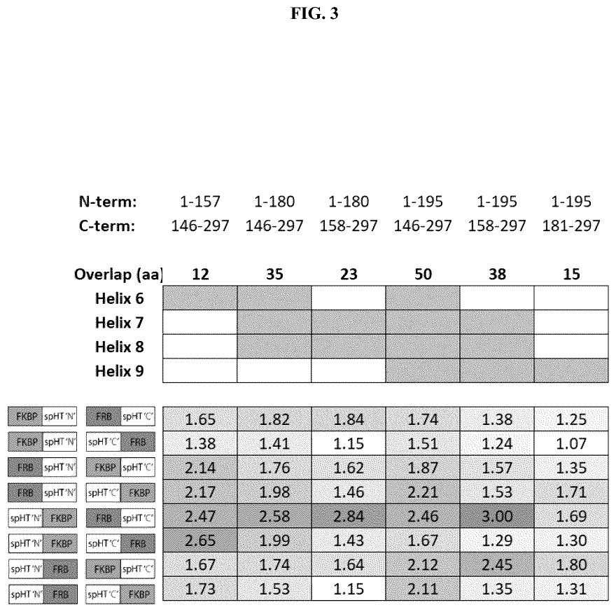

Six out of 22 spHT FRB/FKBP designs exhibited ≥2-fold fluorescence signal increase in the presence of rapamycin (spHT 80, 133, 145, 157, 180, and 195), with up to 4.7-fold induction noted for the combination of [1-195]-FKBP+[196-297]-FRB (FIG. 2). The corresponding cpHT 195 was the most thermostable of all variants in the circular permutation screen (at around 7° C. higher Tₘ than HT). All but one spHT hit (spHT 80) were located in the lid subdomain of HT, which comprises the region of the sequence covering residues 133-216. Among the spHT hits, there were multiple orientations of FRB and FKBP that allowed reconstitution of activity. Generally, fusion combinations in which FKBP was at the C-terminus of either fragment performed the best.

In addition to blunt spHT fragment combinations (in which all HT residues are present exactly once), several “gapped” combinations (e.g., having deletions relative to a parent sequence) and “overlapped” combinations (e.g., having duplications relative to a parent sequence) were tested (in which certain residues were missing from both fragments or present on both fragments, respectively). The missing or double-represented residues in these combinations were confined to the lid subdomain, specifically, Helix 6, Helix 7, Helix 8, and/or Helix 9. Gapped combinations failed to reconstitute detectable ligand binding activity. Overlapped combinations, however, exhibited reconstitution up to 3-fold over background, (FIG. 3). These results indicate that (a) the lid helices are critical for ligand binding in HT, and (b) the lid subdomain tolerates sequence duplications and may engage in secondary structure swapping, a useful feature for designing biosensors and conformationally dynamic protein switches.

Reversibility of spHT Complementation

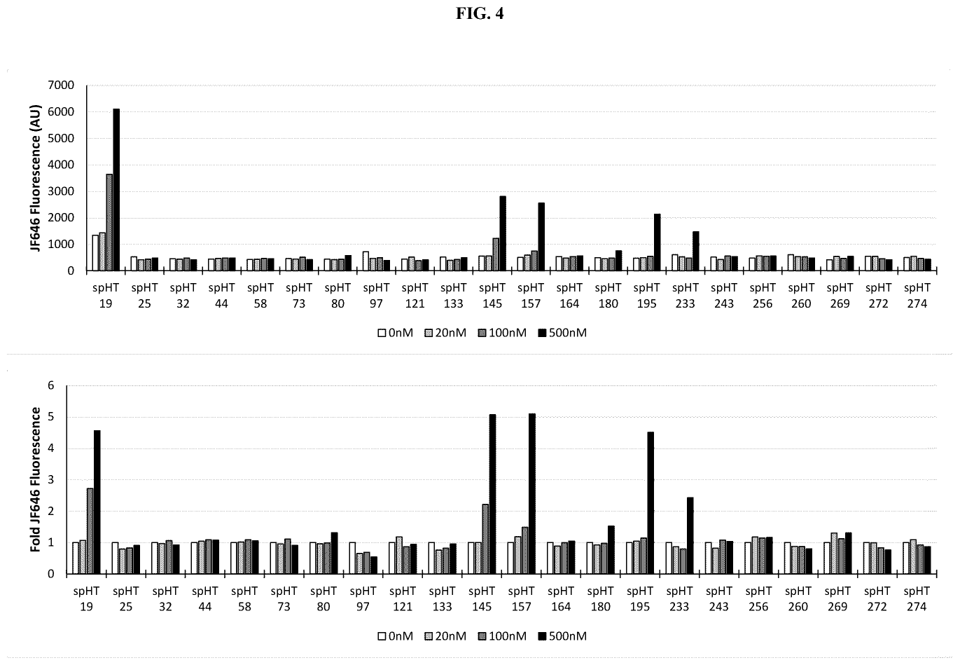

Reversibility is a critical characteristic of bimolecular reporter systems, and experiments were conducted during development of embodiments herein to determine whether the formation of spHT complexes could be reversed, and how this may affect ligand binding and signal dynamics. First, the spHT FRB/FKBP pairs were re-tested with higher concentrations of rapamycin. Several spHT combinations showed sharp increases (up to 5-fold over background) in JF646 fluorescence only when 100 nM or 500 nM rapamycin was added (FIG. 4), indicating that the rapamycin employed in earlier tests may have been insufficient for some spHT designs. spHT 19, 145, 157, 195, and 233 stood out with the highest fold increases in JF646 fluorescence. 500 nM rapamycin was used in subsequent reversibility experiments.

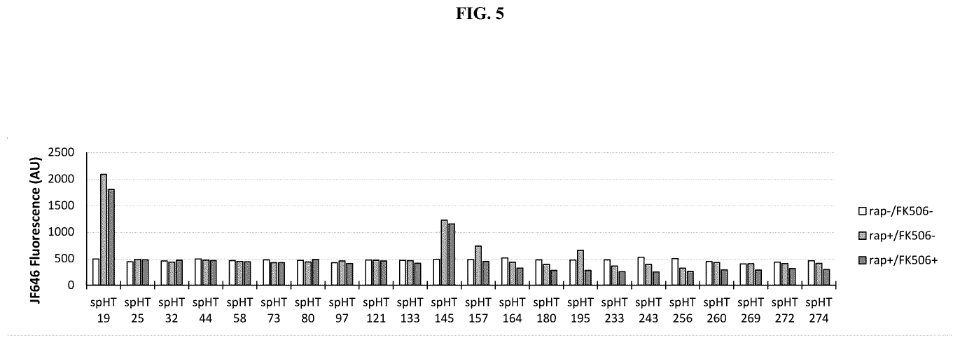

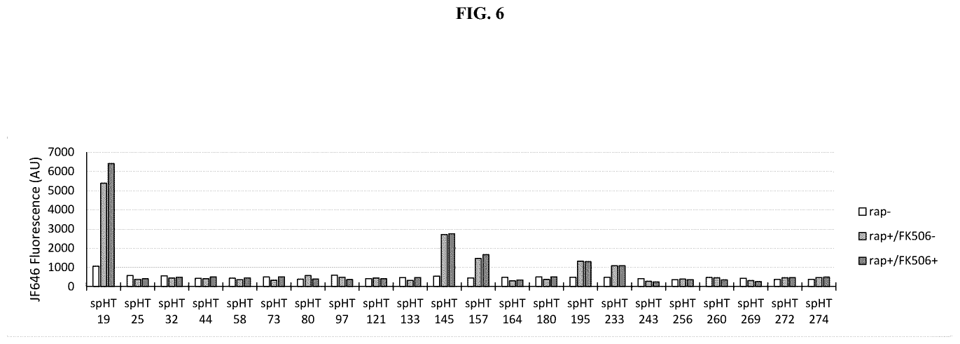

spHT FRB/FKBP fusion combinations were incubated for 24 h with 500 nM rapamycin, then a 31-fold molar excess (10 uM) of the competitive ligand FK506 was added. 24 h later, JF646 was added and allowed to bind for another 24 h (72 h total time elapsed). spHT 19 had slightly less fluorescence compared to its no-FK506 control, and spHT 157, 195, and 233 had only background levels of fluorescence compared to their no-FK506 controls (FIG. 5). However, spHT 145 fluorescence was not decreased relative to its no-FK506 control. That is, rapamycin caused spHT 145 to form an irreversible complex, spHT 19 to form a semi-reversible complex, and spHT 157, 195, and 233 to form reversible complexes.

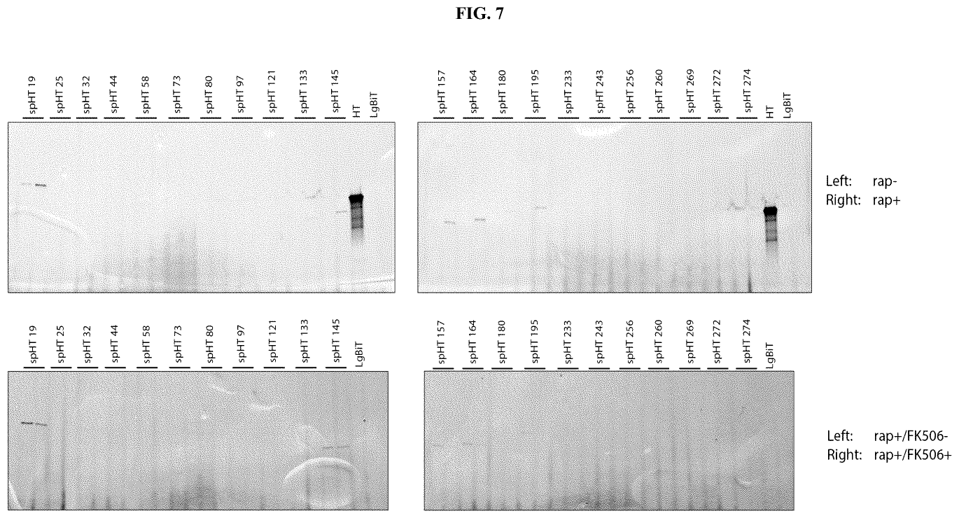

spHT FRB/FKBP fusion combinations were incubated for 24 h with 500 nM rapamycin, 48 h with JF646, then 48 h with 10-fold molar excess of FK506 (FIG. 6). In this case, FK506 failed to reverse the fluorescence development of spHT 19, 145, 157, 195, and 233. That is, JF646 fluorescence did not decrease when the rapamycin was competed out of the FKBP fusion, and the induced dimerization signal was removed. These observations were confirmed using TMR labeling and SDS-PAGE analysis (FIG. 7).

Taken together, these results demonstrate that some spHT fragments (e.g., split sites 145, 157, 195, etc.) may require long periods of close proximity to form complexes, likely because, as spatially separated entities, they form non-complementary, non-native structures and need time to sample many conformations in the presence of their stabilizing partners. However, experiments conducted during development of embodiments herein have demonstrated with N-terminal splits sites (e.g., splits at 19 or 30) that other spHT fragments form detectable complexes in 30 min or less.; Some spHT variants have high affinity and form irreversible (FK506-resistant) complexes, like spHT 145; other complexes are susceptible to FK506 because of their low affinity, like spHT 157, 195, and 233. Complexes that bind to ligand benefit from further stabilization that renders them FK506-resistant spHT complexes may be reversible in their ligand-free state, but can become irreversible in their ligand-bound state.

Quantitative Reconstitution of spHT 19 by Titration with the Short N-Terminal Fragment

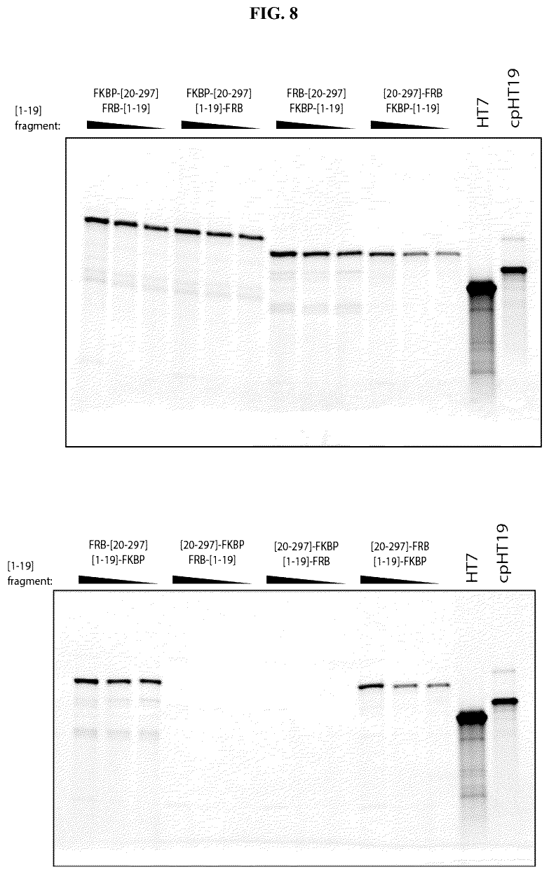

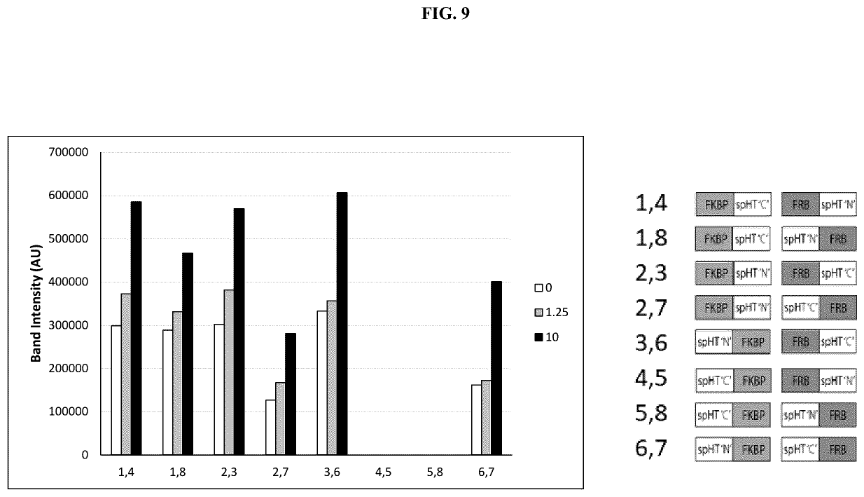

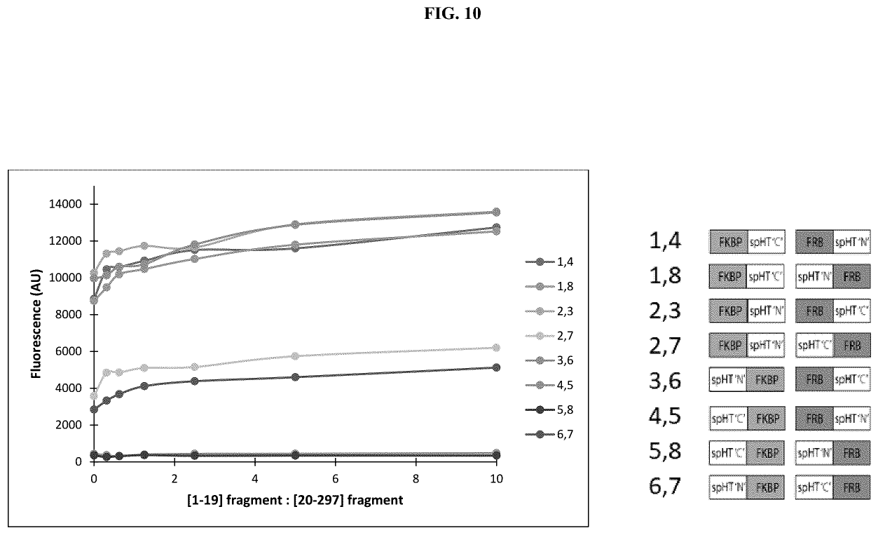

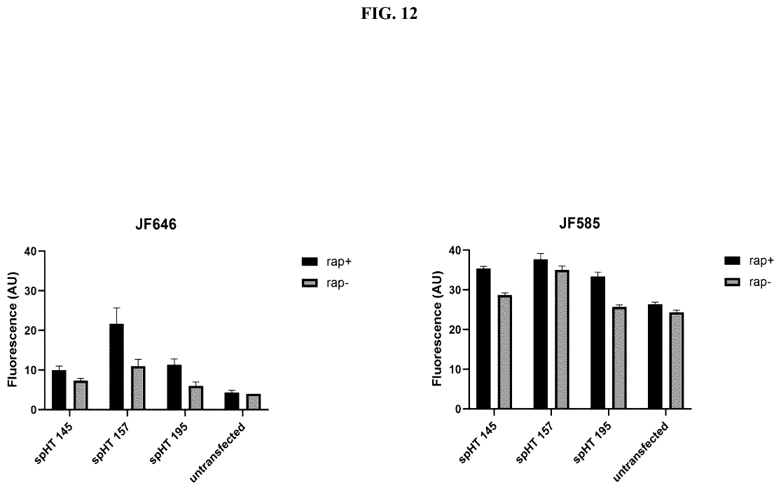

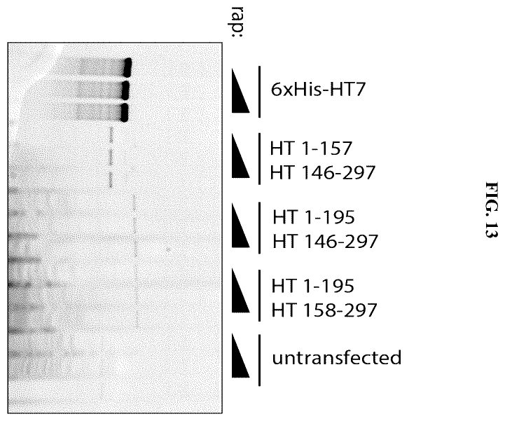

Based on the sensitivity of spHT to rapamycin concentration, it was predicted that fluorescence would also be sensitive to the ratio of spHT fragment concentrations. spHT 19 was used as a test case because the larger C-terminal fragment possesses measurable background activity, and the smaller N-terminal fragment has appeal as a potential peptide tag. The large C-terminal fragment was held constant in all eight spHT 19 FRB/FKBP fusion combinations, while the concentration of the small N-terminal fragment was varied. It was found that by increasing the N:C ratio from 1.25 to 10, TMR labeling efficiency could be increased by >100% depending on the orientation of FRB and FKBP in the fusions (FIGS. 8 and 12). Likewise, JF646 fluorogenic signal could be increased by up to ˜25% at a N:C ratio of 10 (FIG. 10). The greater responsiveness observed in TMR labeling is likely because under the TMR labeling conditions (10 μM substrate), labeling is limited by spHT complex concentration, while under the JF646 labeling conditions (0.1 μM substrate), the substrate concentration is the limiting factor.

These results indicate that the larger C-terminal fragment of spHT can serve as a quantitative, integrated sensor of the smaller N-terminal fragment in the presence of ample ligand and a high affinity partner interaction, such as the FRB-rapamycin-FKBP interaction.

Expression and Activity of spHT Variants in Mammalian Cells

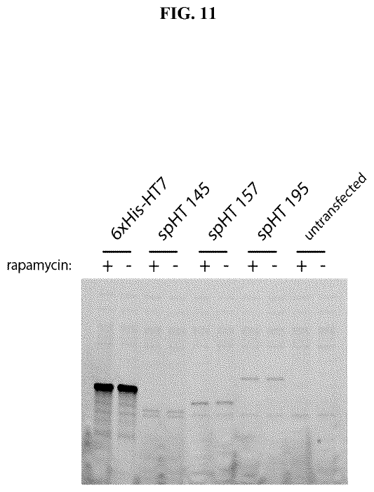

A small subset of spHT variants (spHT 145, 157, and 195) were selected for expression in mammalian cells (HeLa cells). Cells were co-transfected with pF4Ag shuttle vectors encoding spHT fragments as fusions to FKBP and FRB, with FKBP appended to the C-terminal of the first fragment and FRB appended to the C-terminal of the second fragment in each case. HT activity was observed both in lysates (using the non-fluorogenic TMR ligand, FIG. 11) and in live cells (using the fluorogenic JF646 and JF585 ligands, FIG. 12) for all spHT co-transfectants. Activity was visible even in the absence of rapamycin. While the addition of 50 nM rapamycin increased JF646 and JF585 fluorescence yield in live cells by up to ˜2-fold, it had no apparent effect on band intensity in TMR-labeled SDS-PAGE. That is, TMR labeling occurred equally (and significantly) with or without rapamycin.

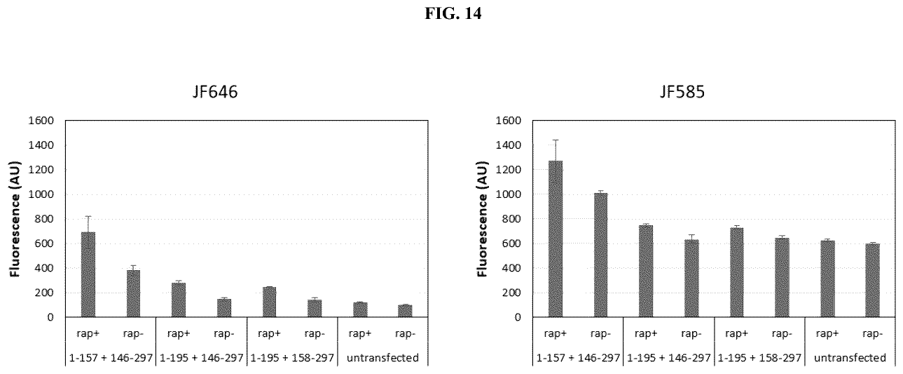

Similar results were obtained with overlapped spHT combinations in HeLa cells (FIGS. 13 and 14). These results indicate that (a) co-expression of spHT fragments allows the fragments to spontaneously complement in a manner not observed during independent expression and subsequent mixing, indicative of possible co-translational folding; (b) in such pre-assembled spHT complexes, rapamycin can assist the formation of properly folded lid subdomains by stabilizing the interaction of lid helices, or reducing their conformational degrees of freedom; (c) such actions of rapamycin may increase fluorogenic ligand (e.g., JF646 and JF585) signal without increasing total ligand binding (as measured by saturation with TMR ligand), because the total concentration of spHT complexes is not significantly changed.

### Example 3

### Internal Split Sites

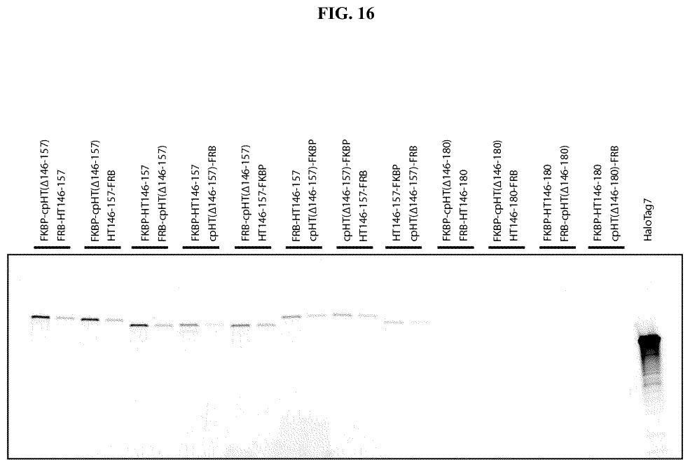

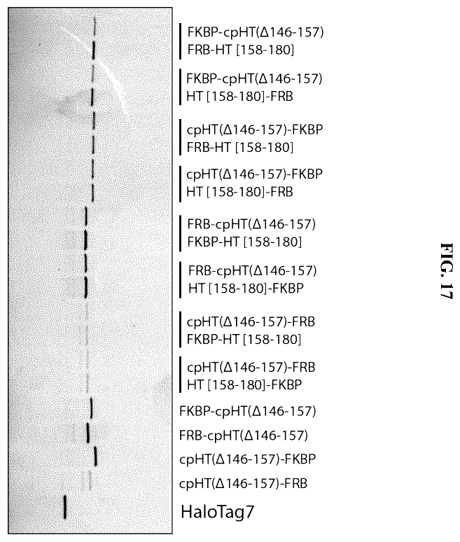

Several split sites located in the lid domain were further examined for complementation as circular permutations that split peptides internally off the lid domain (i.e., removing an internal helix and testing for complementation) including configurations that contained gapped and overlapping residues in the complementing peptides. Since it was previously observed that several circularly permuted HaloTag variants missing residues in the region of 146-195 of the lid domain were expressed and soluble in E. coli, it was tested whether complementation could be observed when residues corresponding to the missing fragments were reintroduced. It was found that Rapamycin-dependent complementation activity could be observed when cpHaloTag missing residues 146-157 (cpHT(A146-157), which lack the Helix 6 of the lid domain) fused to FRB or FKBP was paired with the cognate 146-157 peptide (HT146-157) as fusions to FRB or FKBP (FIG. 16). Complementation was not observed with larger lid domain deletions in the cpHaloTag (cpHTΔ146-180) when attempting to pair with the cognate missing peptide fragment.

Since the cpHT(Δ146-157) internal deletion fragment was functional when the complementary missing residues were reintroduced as a separate peptide, it was tested whether other peptides comprising various other lid domain residues could show Rapamycin-dependent complementation activity. FIG. 17 shows Rapamycin-induced enhancement of activity when the cpHT(Δ146-157) fragment was paired with the HT(158-180) peptide as fusions to FRB or FKBP. This pair of constructs shares an overlap in the 158-180 residue region and a gap in the 146-157 region of the complex, but was still functional for activity in the assay and responsive to Rapamycin.

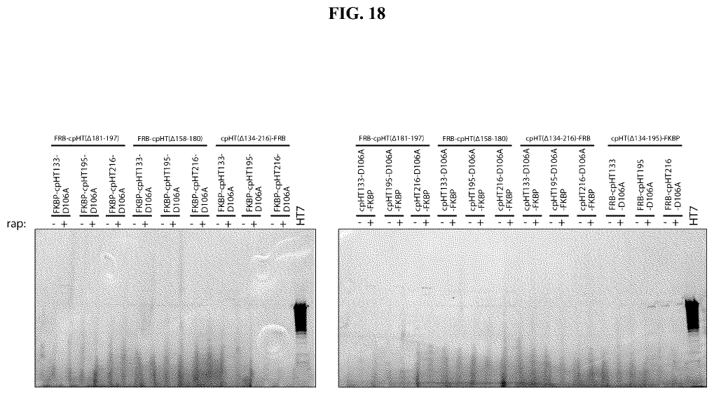

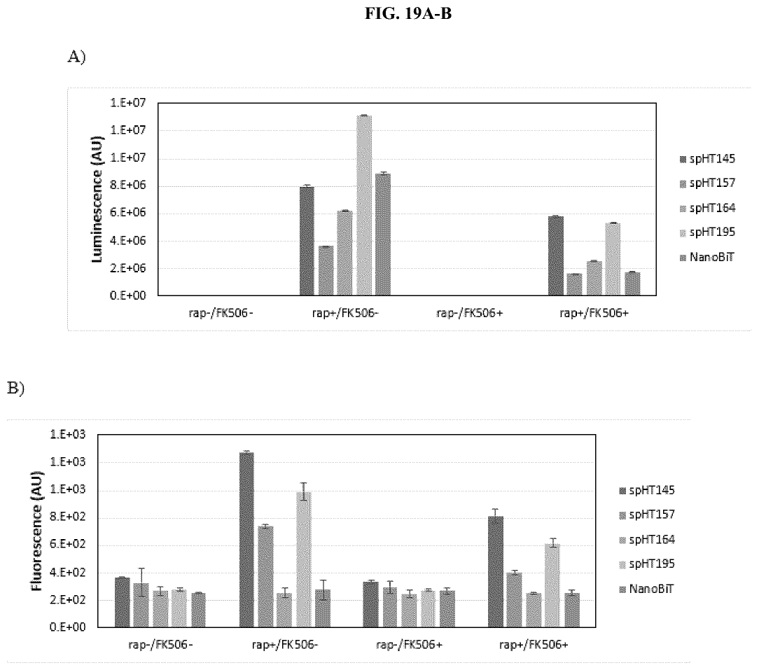

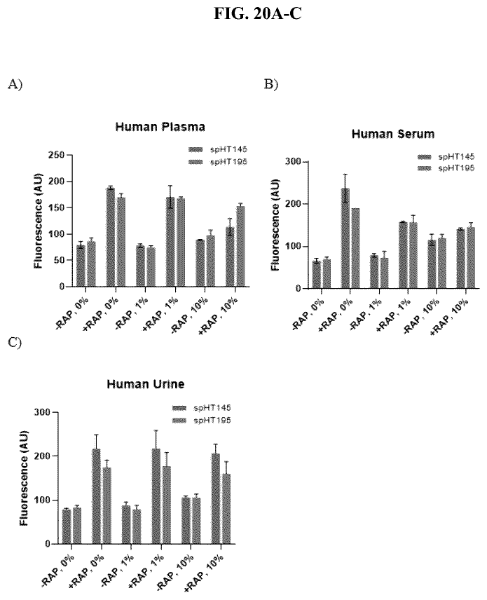

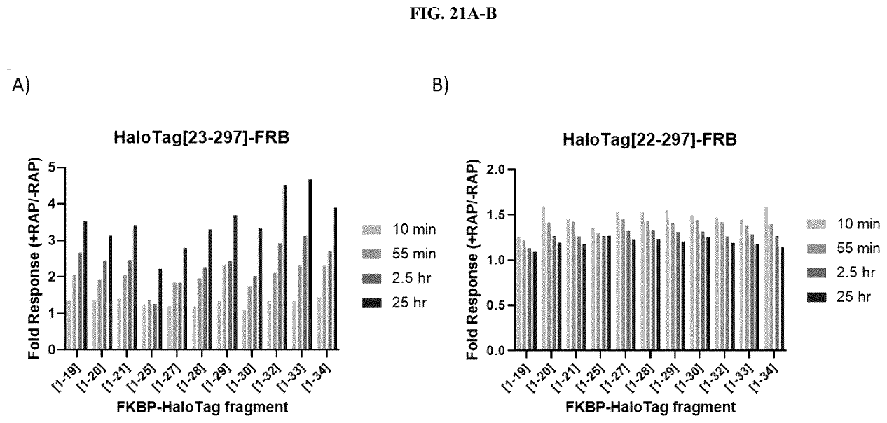

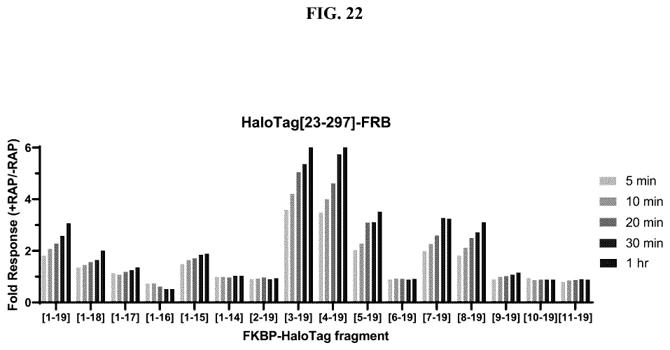

Following the experiments showing that split cpHaloTag variants lacking fragments of the lid domain (specifically Helix 6; residues 146-157) could be complemented with peptides comprising smaller lid domain fragments, either corresponding to the missing residues or those with overlap and gapped configurations, it was tested whether complementation could also occur by donation of lid domain residues through domain swapping. Domain swapping is a phenomenon where two polypeptides exchange similar folded domains that can recapitulate the monomeric structure of each when occurring between the similar (often the same) protein. In the case of HaloTag, it has been shown that its lid subdomain can “swap” among monomers, creating a dimeric structure where each monomer is comprised of its own core a/b-hydrolase domain and its partner's lid domain. Since the function of HaloTag relies on the proper folding of its lid domain to bind the chloroalkane substrate, it was reasoned that cpHaloTag variants lacking fragments of the lid domain could have their activity restored if another cpHaloTag construct could swap or donate the missing residues to form a complete HaloTag structure. In order to detect activity only when domain swapping occurs, the D106A mutation was made in the domain “donor” construct in the pairs shown in FIG. 18. The D106A mutation eliminates covalent attachment of the chloroalkane (so it would not be detected on gels). However, since those mutant cpHaloTag variants still retain their lid domain residues, they are capable of swapping them into the split cpHaloTag variants to complement their missing residues, restore their activity, and subsequently enable labeling with a TMR HaloTag ligand detectable following SDS-PAGE. FIG. 18 shows success in identifying constructs that can domain swap and complement split HaloTag fragments under these conditions, facilitated by their fusion to FRB or FKBP and inclusion of Rapamycin. This experiment shows that cpHaloTag variants missing fragments of their lid domain cpHT(Δ146-157) can form a complementation system through domain swapping with other cpHaloTag variants that have termini in the lid domain and can donate the necessary residues.

LgBiT and SmBiT tags on fragments of split HaloTag fused to FRB/FKBP were used to measure complementation and reversibility of each complex in a fluorescence-independent manner. NanoBiT detection of fragment complementation closely matched the pattern of activities associated with JF646 HaloTag ligand labeling. In the absence of Rapamycin, low luminescence and JF646 labeling was detected, but upon addition of Rapamycin both signals increased significantly, indicating that complex formation and restoration of enzymatic activity were dependent on facilitation though the FRB:FKBP interaction. The addition of FK506, an inhibitor of the FRB:FKBP interaction, to reactions showed a decrease both luminescence and fluorescence signals for all constructs after their Rapamycin-dependent complementation, demonstrating that these split HaloTag fragments are physically and functionally reversible.

Robustness of the internal split HaloTag complementation systems to human body fluid matrices was tested in order to assess their utility for diagnostic or clinical applications. Both the spHT145 and spHT195 showed resistance to each matrix up to the 10% limit that was tested, retaining their Rapamycin-dependent complementation and activity. This experiment demonstrates that these split HaloTag fragments were tolerant of human fluid body matrices and could be envisioned as a technology for detecting molecular proximity or binding in diagnostic or clinical assays.

### Example 4

### N-Terminal Split Sites

Experiments were conducted during development of embodiments herein to test combinations of N-terminal split HaloTag fragments to determine if they can be induced to complement as FRB or FKBP fusions. The role of sequence overlap in determining performance was examined. A range of small peptide-sized, N-terminal fragments could be observed to show a Rapamycin-dependent response in activity with JF646 HaloTag ligand. Since the larger fragment was comprised of residues 22-297 or 23-297, many of the small fragments tested have either gaps or overlaps in their sequences. This demonstrated complementation with these N-terminal split fragments across a range of sequence variability and lengths.

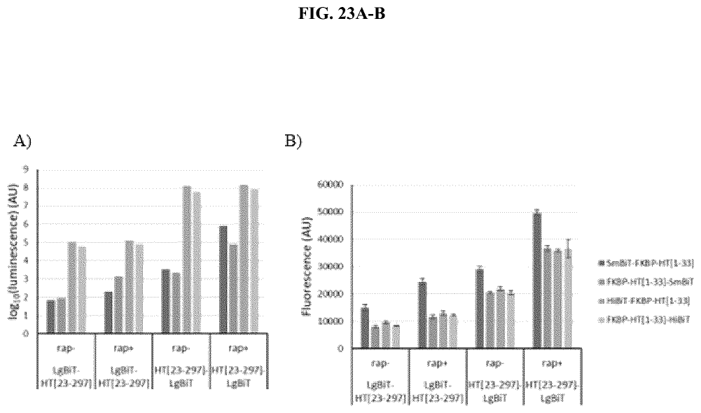

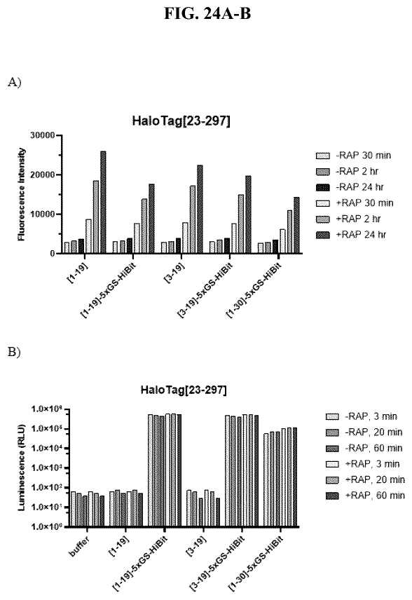

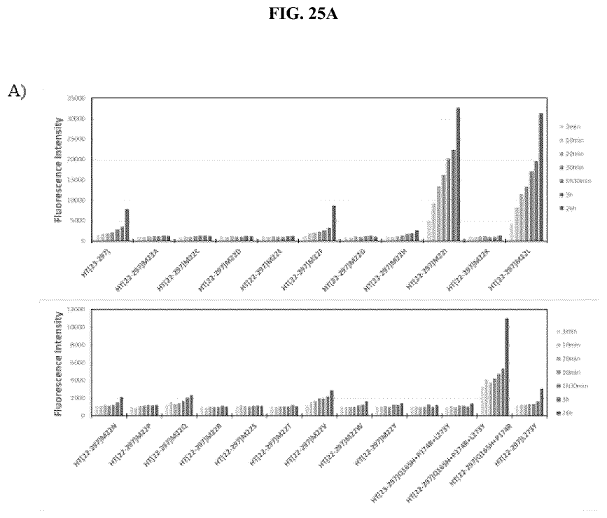

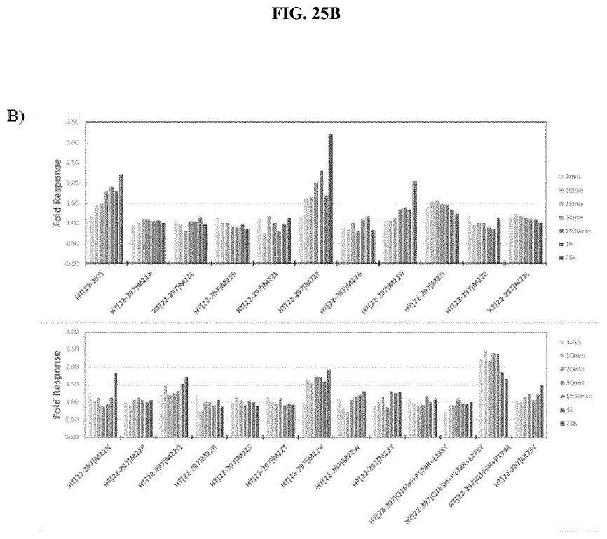

N-terminal split HaloTag system was optimizable through systematic evaluation of truncations of the smaller HT(1-19) fragment. FIG. 22 shows that truncation of the first 2-3 N-terminal residues in particular enhances the fold response of the system to Rapamycin. Complementation activity was demonstrated with fragments as small as 11 amino acids (HT(8-19)).

The ability to detect complementation of a N-terminal split HaloTag fragment independent of its activity by fusion to NanoBiT components was tested. A 100-250-fold increase in luminescence was observed upon addition of Rapamycin, which corresponded to increases in labeling with JF646 in separate assays, indicating that both fragment complementation though physical proximity and also enzymatic activity could be detected for the asymmetric N-terminal split HaloTag fragments.

Other N-terminal split HaloTag fragments were functional in dual tag configurations with HiBiT. When HiBiT was appended to multiple different, N-terminal, small HaloTag fragments, both HaloTag activity through binding of JF646 ligand and NanoBiT activity with the HiBiT tag could simultaneously be detected (in different reactions). This demonstrated that these tags could be used in tandem for making multiple measurements from a single system such that users could append this dual tag for multiple uses in both luminescence and fluorescence.

Given the differences between the HT(22-297) and HT(23-297) large fragments, the role of the Met22 position was examined through site saturation and mutation to all other amino acids. Amino acids at position 22 that improve brightness (M22I or M22L, for example) were identified, and those that improve the fold response of the system (M22F). It was also observed that introduction of mutations of “HaloTag9” (Q165H+P174R) significantly enhanced the labeling speed of the system when added to the HT(22-297) large fragment. These experiments demonstrated that mutations can be introduced to both the large and small fragments of these split HaloTag variants to improve system performance.

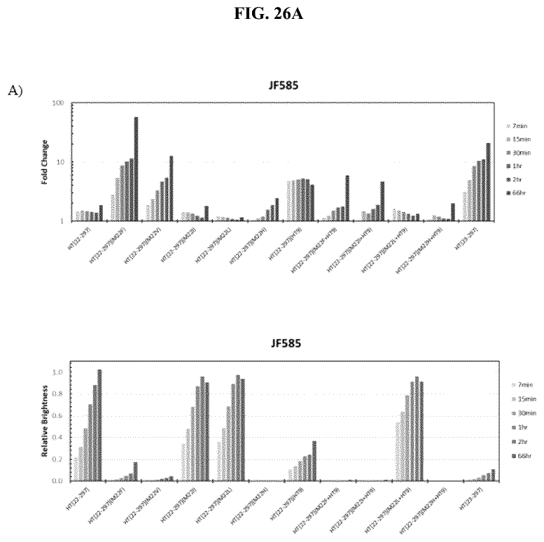

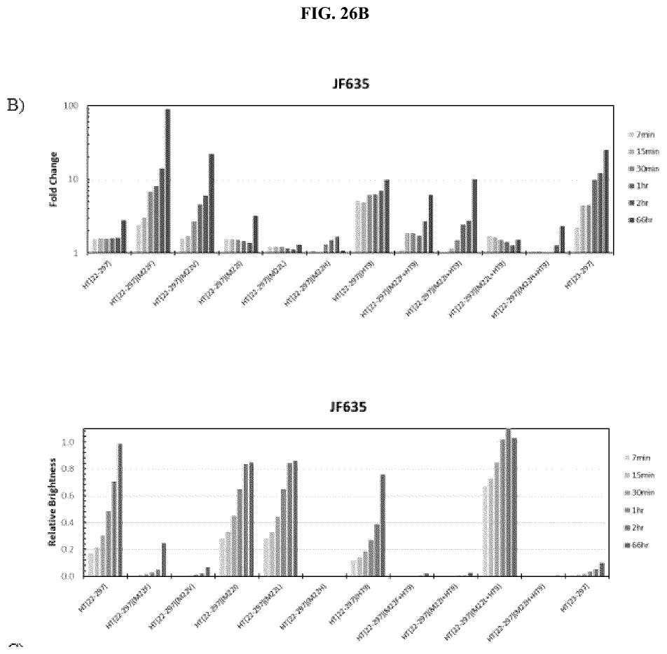

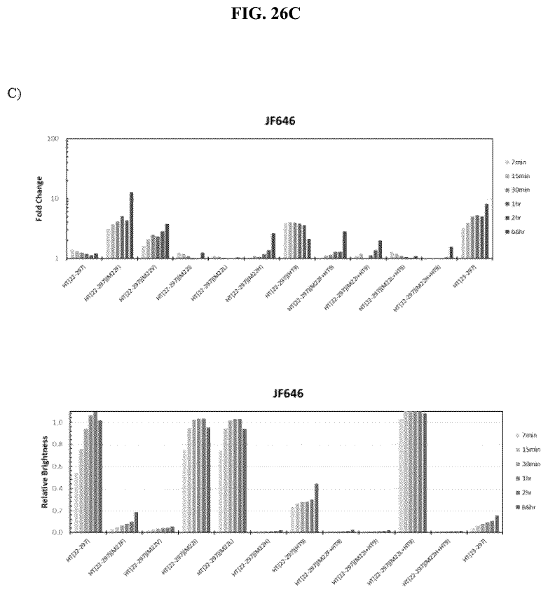

Given that the mutations in each small and large fragment of the N-terminal HaloTag splits resulted in improvements in brightness and fold response with the JF646 ligand, they were tested with other Janelia Fluor HaloTag ligands (FIG. 26). This experiment demonstrated that mutations in each of the N-terminal split fragments can modulate kinetics of labeling with different HaloTag ligands. In addition, it showed that given enough time, out to 66 hours, some of the configurations continue to increase in brightness and fold response, resulting in responses for some as much as 60-fold. It also showed that several configurations achieved equivalent brightness to full-length HaloTag7 with their relative brightness at 1.0 or above the HaloTag7 control.

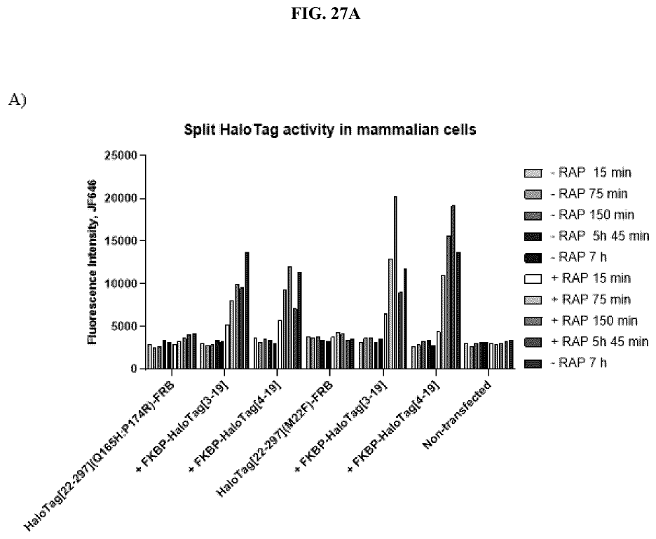

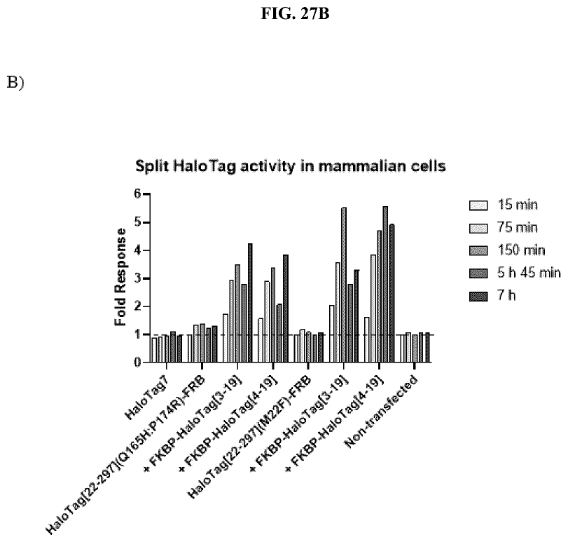

The experiments in FIG. 27 detection of a Rapamycin-dependent response in split HaloTag activity in live mammalian cells. The response could be detected for multiple small HaloTag fragments (HT(3-19), HT(4-19)) with different large HaloTag fragments. System performance was optimized by introduction of mutations into the HT(22-297) large fragment, either the Q165H+P174R double mutant or M22F mutations as shown. The fold responses (labeling speed after pre-incubation with Rapamycin) were detectable within 15 minutes, indicating that these split HaloTag configurations are suitable for live cell-based assays that detect many biological processes in real time.

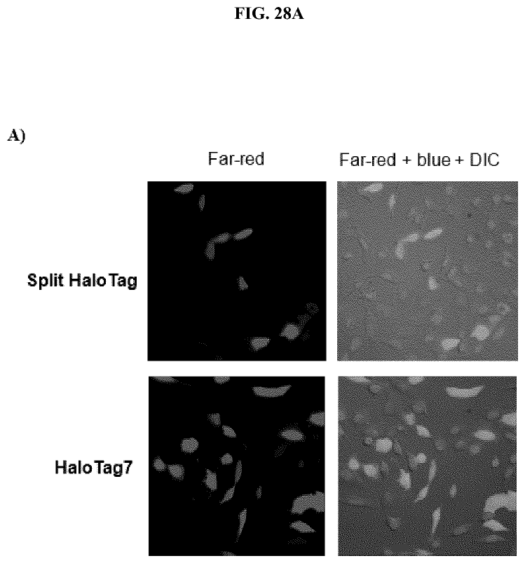

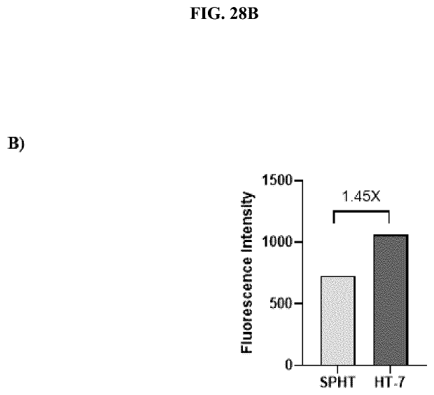

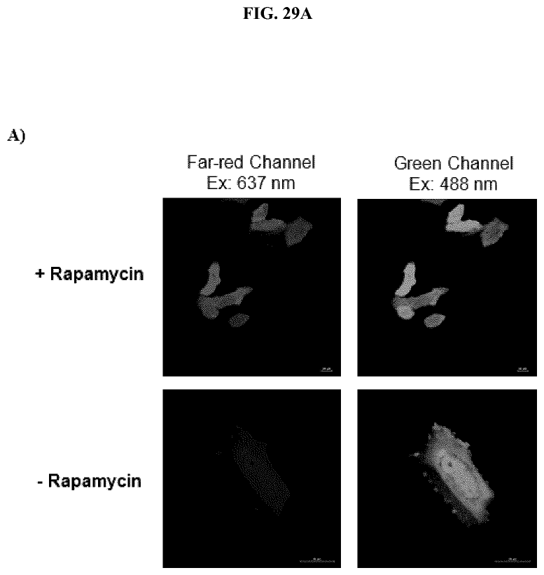

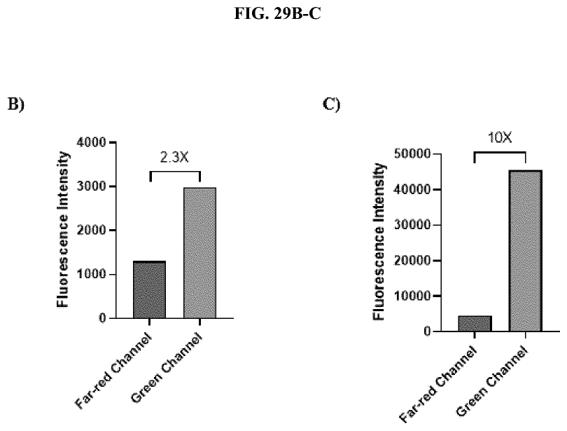

Experiments also demonstrated the functionality and utility of split HaloTag systems in live cell fluorescence microscopy. This is a desirable modality for detection, particularly given the development and use of the Janelia Fluor HaloTag ligands for advanced cell imaging applications such as STED or STORM. Experiments demonstrated that split HaloTag configurations have comparable brightness to full-length HaloTag7 (FIG. 28), and that the fluorescence signal of the live cells was dependent on the addition of Rapamycin to facilitate the interaction of the split HaloTag fragments (FIG. 28), highlighting its utility as a reporter for molecular interactions inside live cells.

### Example 5

### Use of Synthetic Peptides to Measure Split HaloTag Complementation

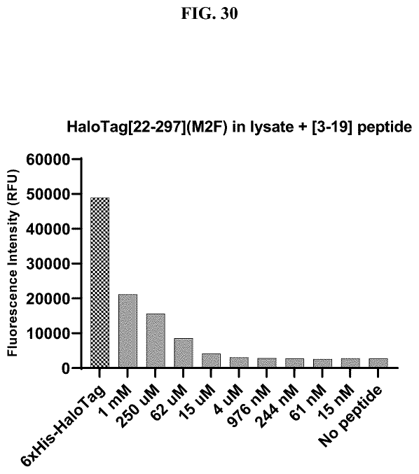

Experiments conducted during development of embodiments herein demonstrated that a synthetic peptide version of the HaloTag[3-19] fragment can be used, and complementation observed with the HaloTag[22-297](M2F) fragment (FIG. 30). At the highest concentration of peptide tested, 1 mM, saturation of the fluorescence signal was not observed, indicating that the affinity between the split HaloTag fragments is likely >100 micromolar, which is expected to be a suitable range for protein:protein interaction studies in vivo since their low affinity requires fusion to interacting partners to form a complemented complex.

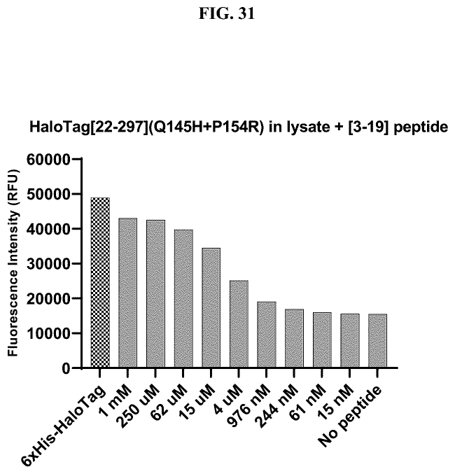

Experiments additionally demonstrated that a synthetic peptide version of the HaloTag[3-19] fragment can be used, and complementation observed, but with a different variant of the LgHT, HaloTag[22-297](Q145H+P154R). This LgHT variant is more stable and expresses better, leading to higher complemented signal, although lower fold response due to its higher “background” or uncomplemented signal (FIG. 31). This LgHT mutant does appear to approach saturation of the fluorescence signal, indicating that the affinity between the split HaloTag fragments is approximately 1 micromolar, which might be considered higher affinity than would be ideal for protein:protein interactions studies in vivo, although complementation in mammalian cells using this LgHT variant has been observed, indicating it can be used for the purpose.

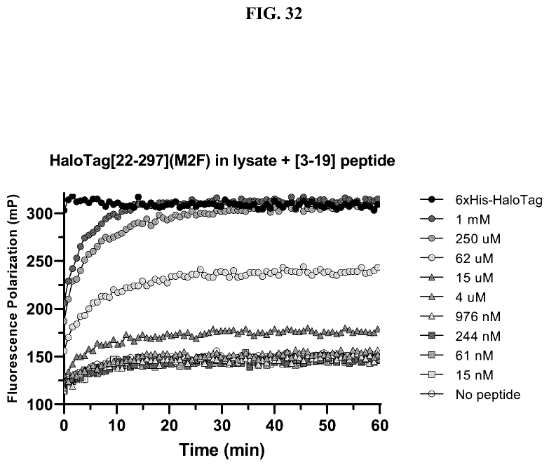

A different HaloTag ligand (TMR) and fluorescence polarization assay format can be used to measure complementation with synthetic peptides (FIG. 32). The relative kinetic rate of labeling for HaloTag[22-297](M2F) at different levels of complementation with peptide is demonstrated. At high peptide concentrations, the complementation with the peptide results in greater labeling rates.

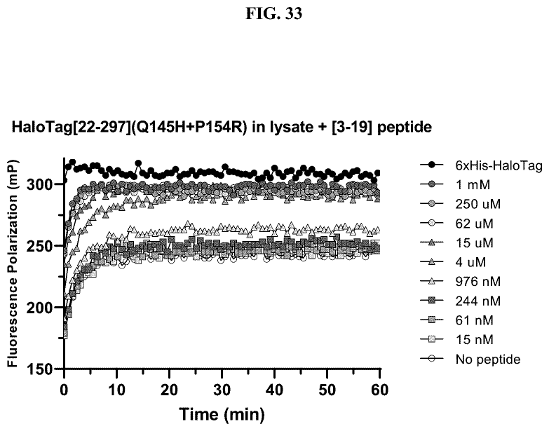

The LgHT variant, HaloTag[22-297](Q145H+P154R), was also tested with the HaloTag ligand (TMR) in the fluorescence polarization assay format to measure complementation with synthetic peptides (FIG. 33). The relative kinetic rate of labeling is higher for this mutant without peptide due to its greater intrinsic stability and expression in E. coli lysates. In the presence of peptide, it is able to achieve faster labeling rates relative to the HaloTag[22-297](M2F) mutant.

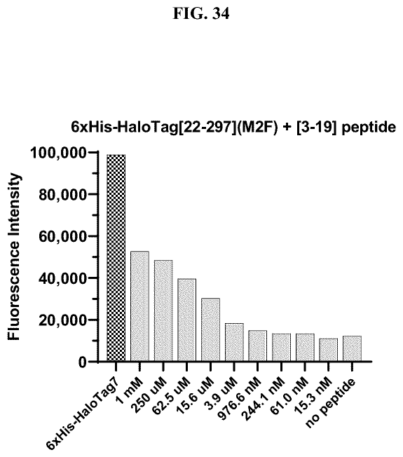

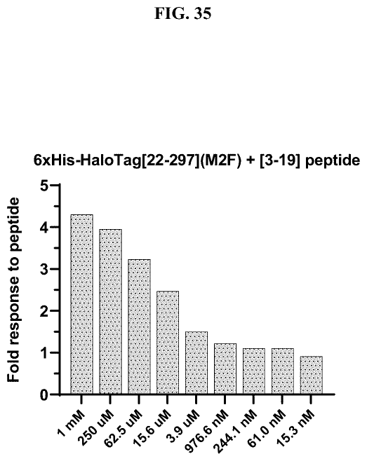

The use of a fully purified split HaloTag system—6×His-HaloTag[22-297](M2F) and synthetic HaloTag[3-19] peptide—are able to complement each other in vitro, resulting in an increase in fluorescence intensity following complementation and labeling with JF646 ligand (FIG. 34). The fold response to varying concentrations of synthetic HaloTag[3-19] peptide for the purified 6×His-HaloTag[22-297](M2F) relative to the uncomplemented reaction lacking peptide was calculated (FIG. 35).

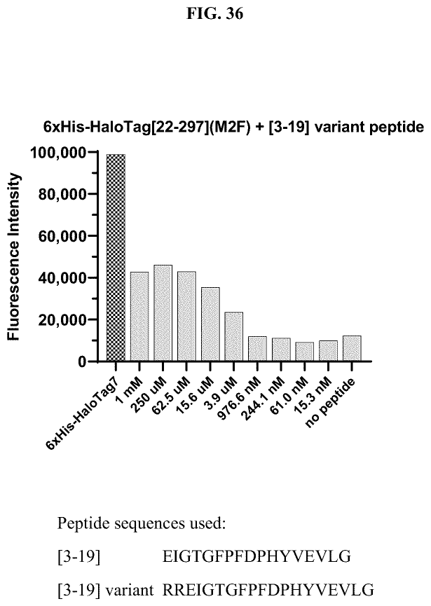

A fully purified split HaloTag system, with the synthetic peptide modified by the addition of two consecutive arginine residues to the N-terminus of the HaloTag[3-19] sequence, was used in an attempt to improve the solubility of the peptide. Although an increase in peptide solubility was not observed, it demonstrated that the N-terminus of the peptide can be modified with additional residues while retaining function of the split HaloTag system (FIG. 36).

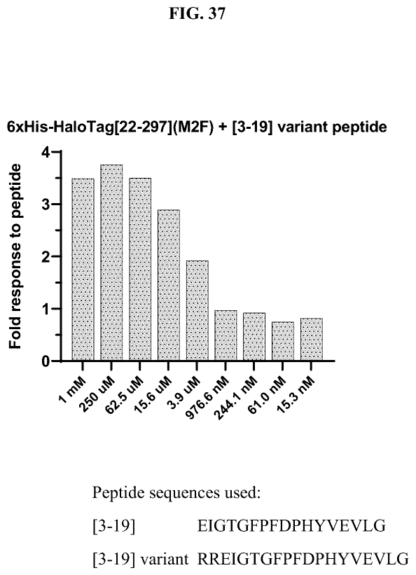

Experiments were conducted during development of embodiments herein to determine the fold response to varying concentrations of the variant synthetic HaloTag[3-19] peptide for the purified 6×His-HaloTag[22-297](M2F) relative to the uncomplemented reaction lacking peptide (FIG. 37).

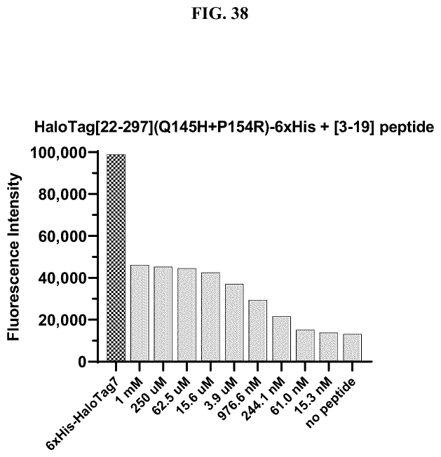

A fully purified split HaloTag system, but with a variant of the LgHT, 6×His-HaloTag[22-297](Q145H+P154R) and synthetic HaloTag[3-19] peptide, was able to complement with each other in vitro, resulting in an increase in fluorescence intensity following complementation and labeling with JF646 ligand (FIG. 38). This variant of LgHT does saturate its fluorescence intensity at higher peptide concentrations, indicative of a higher apparent affinity for the peptide.

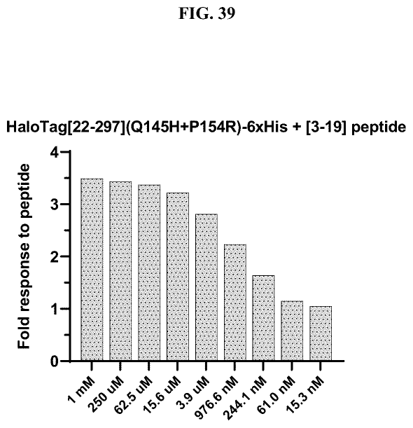

Experiments were conducted during development of embodiments herein to demonstrate the fold response to varying concentrations of synthetic HaloTag[3-19] peptide for the purified 6×His-HaloTag[22-297](Q145H+P154R) relative to the uncomplemented reaction lacking peptide FIG. 39). Due to its higher affinity, this variant LgHT is able to show measurable fold responses to lower concentrations of the peptide, down to the mid-nanomolar range, suggesting it could be suitable as a stand-alone SmHT detection system, assuming the assay conditions are sufficient for complementation.

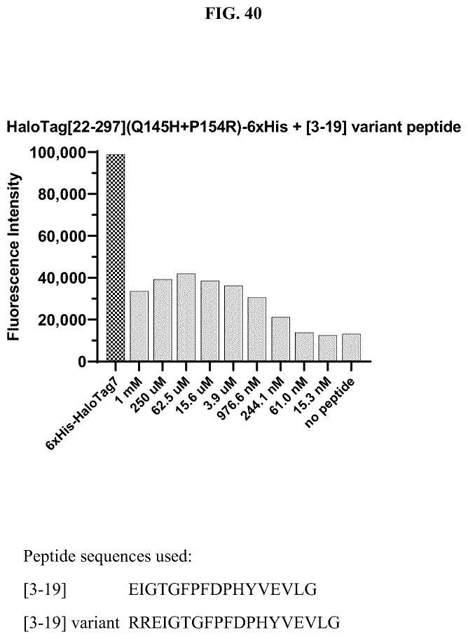

Experiments were conducted to demonstrate the use of a purified split HaloTag system with a modified synthetic peptide that adds two consecutive arginine residues to the N-terminus of the HaloTag[3-19] sequence (FIG. 40). The N-terminus of the peptide can be modified with additional residues and retain function of the split HaloTag system, in this case, using the HaloTag[22-297](Q145H+P154R) variant.

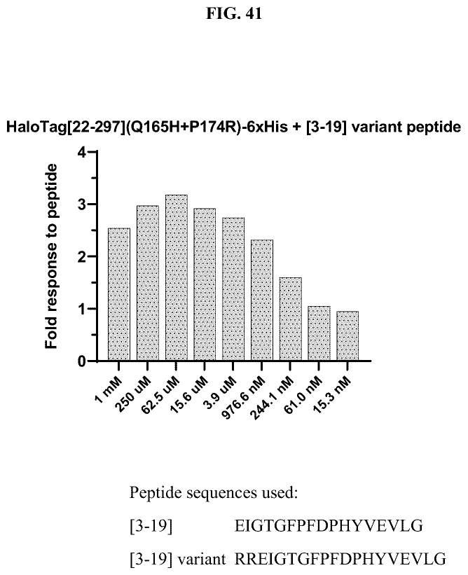

The fold response to varying concentrations of the variant synthetic HaloTag[3-19] peptide for the purified 6×His-HaloTag[22-297](Q145H+P154R) relative to the uncomplemented reaction lacking peptide (FIG. 41). Relative to the HaloTag[22-297](M2F) comparator, this LgHT variant shows a higher fold response at lower peptide concentrations, potentially useful for detection of the variant SmHT peptide as a detection reagent.

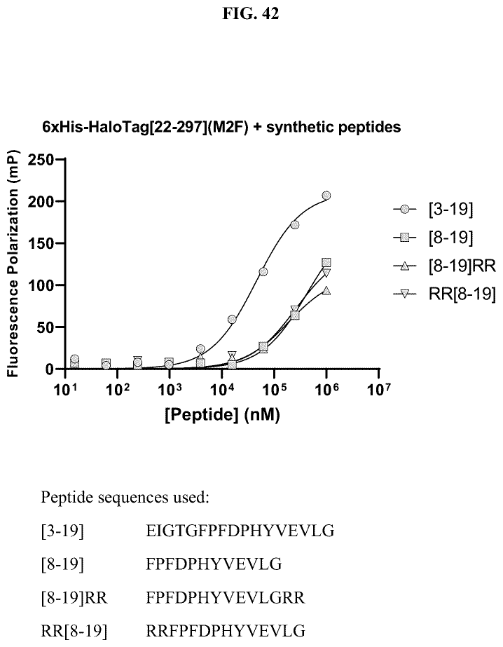

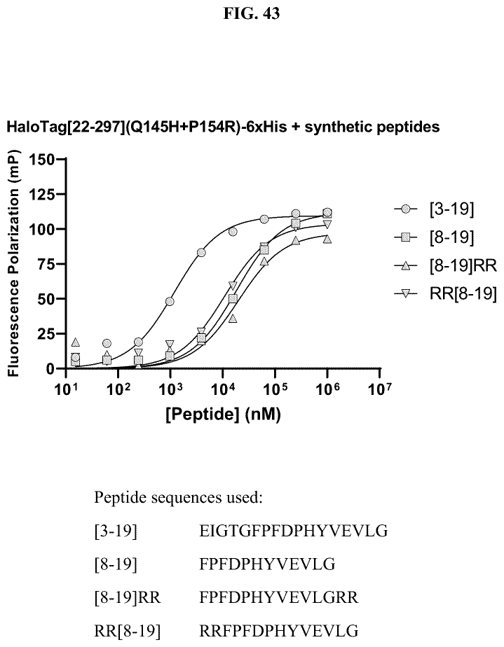

This purified system shows the successful detection of shorter peptides based on residues HaloTag[8-19], with N- or C-terminal arginine addition (FIG. 42). The shorter peptides show a lower affinity than the HaloTag[3-19] peptide. This demonstrates that a shorter peptide can be used for complementation in the split HaloTag system, and that sequence additions to the shorter sequence can be tolerated, potentially to optimize the system further. This purified system also shows the successful detection of shorter peptides based on residues HaloTag[8-19], with N- or C-terminal arginine addition, in this case using the variant LgHT, HaloTag[22-297](Q145H+P154R) (FIG. 43). The shorter peptides show a lower affinity than the HaloTag[3-19] peptide, however, since this LgHT variant has higher affinity for the full length and shorter peptides

### Example 6

### Mutagenesis of the HaloTag[3-19] or “Small HaloTag” Sequence

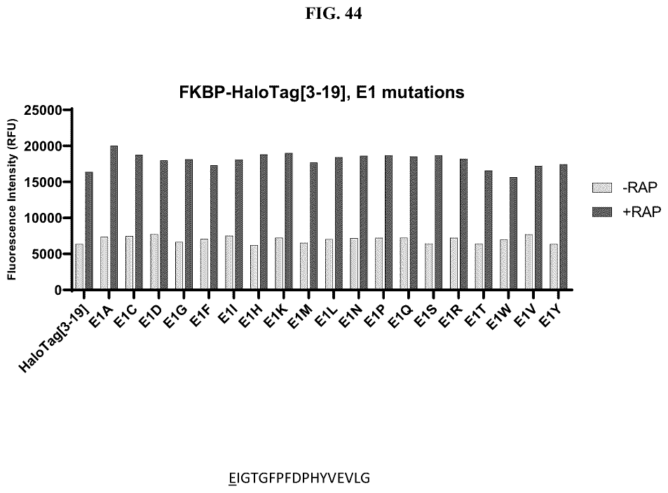

Experiments were conducted during development of embodiments herein to test all possible single mutations in the HaloTag[3-19] fragment. Expression and activity data of the HaloTag[3-19] variants is provided in Table 1. FIG. 44-60 represent the 19 amino acid changes at an individual position (numbered 1-17). The HaloTag[3-19] mutants were expressed as fusions to the C-terminus of FKBP and tested for facilitated complementation with the HaloTag[22-297](M2F)-FRB fusion via the addition of Rapamycin in E. coli lysates. These experiments helped identify positions in the HaloTag[3-19] fragment that are tolerant to mutations and enabled building of multiple mutations into the fragment. The effects of combining well tolerated mutations at each position together are shown, starting with double/triple/quadruple/etc. mutations, and eventually reaching designs where all 17 positions can be mutated simultaneously in functional variants that have 0% identity with the starting HaloTag[3-19] sequence. Most interactions with the large fragment occur through the main-chain, beta-strand interactions and do not rely on sidechains. For the few sidechains that do participate in the interaction, conservative mutations to change the residue but preserve the characteristics of the sidechain interaction can be made, such as a phenylalanine to tyrosine mutation, preserving the sidechain hydrophobicity. Therefore, a functional small HaloTag fragment with all 17 positions mutated can be achieved.

Mutation of residue E1 of the HaloTag[3-19] sequence showed no detrimental mutations and several potential beneficial ones (E1A, E1H, E1K) (FIG. 44).

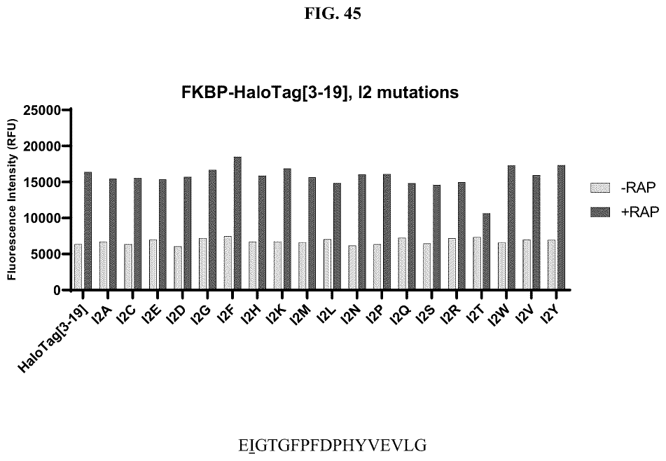

Mutation of residue I2 of the HaloTag[3-19] sequence showed beneficial mutations with larger hydrophobic sidechain such as I2F, I2W, and I2Y (FIG. 45). The I2T mutation was the only one that showed a significant loss of performance in the assay.

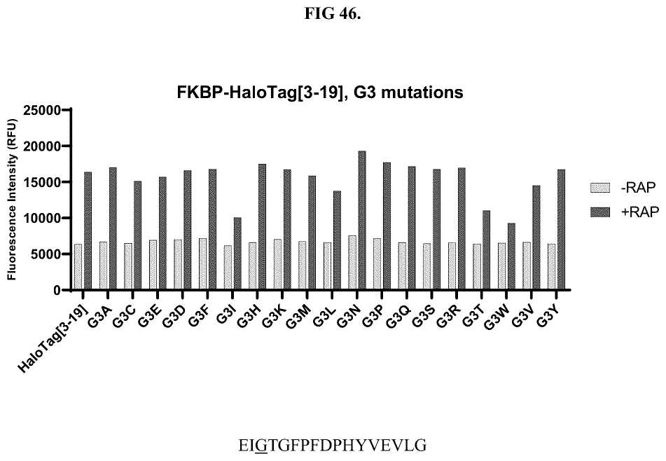

Mutation of residue G3 of the HaloTag[3-19] sequence showed improvement with the G3N mutation and several detrimental mutations: G3I, G3T, and G3W that had significant loss of performance (FIG. 46).

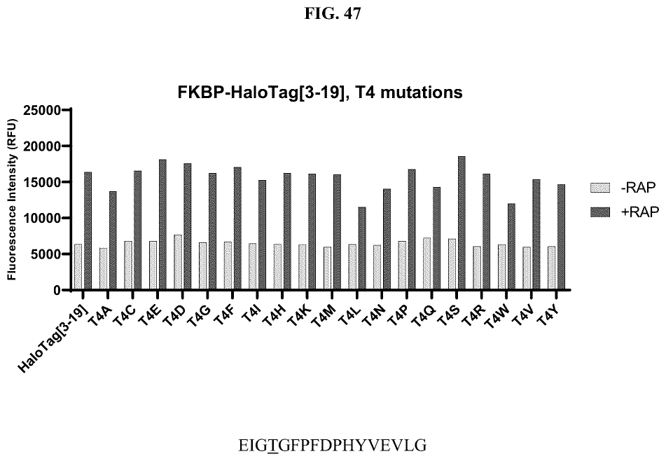

Mutation of residue T4 of the HaloTag[3-19] sequence showed some improvement with T4S and T4E mutations and some detrimental effects from the T4L and T4W mutations (FIG. 47).

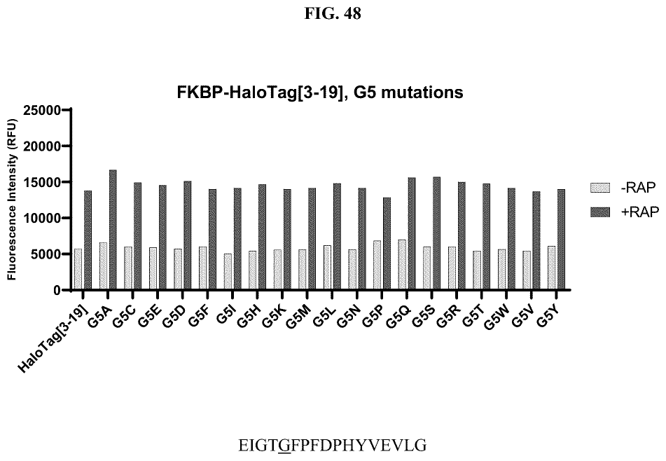

Mutation of residue G5 of the HaloTag[3-19] sequence showed generally good tolerance across all amino acid changes (FIG. 48).

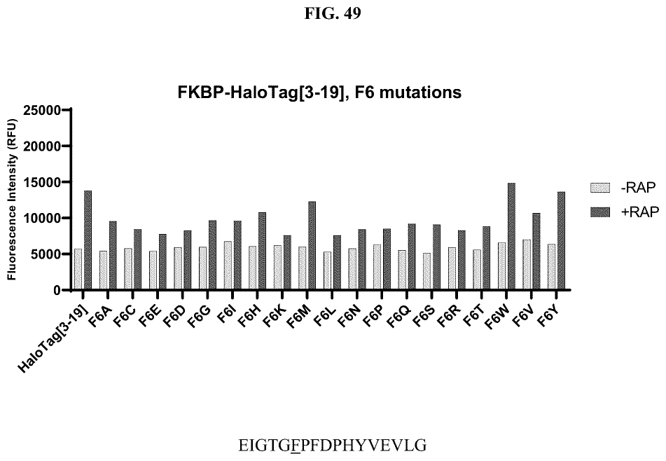

Mutation of residue F6 of the HaloTag[3-19] sequence showed this residue to be highly sensitive to mutation (FIG. 49). Most mutations were detrimental, with the exception of F6M, F6W, and F6Y that showed performance similar to the unmutated protein. This is an example that shows preserving the hydrophobic nature (see also Ile and Val mutations) and especially ring structure (see also Histidine mutation) of the sidechain to be beneficial for interaction with the large fragment.

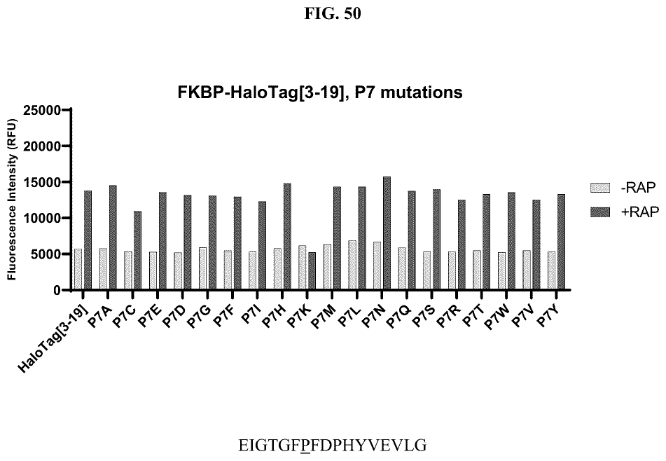

Mutation of residue P7 of the HaloTag[3-19] sequence showed improvements with P7N and less so P7H or P7A (FIG. 50). The mutation to P7K appeared to be significantly detrimental to activity. As shown in later figures, mutations such as P7N are well tolerated as a single mutation but generally result in lower activity when combined with other mutations in HaloTag[3-19].

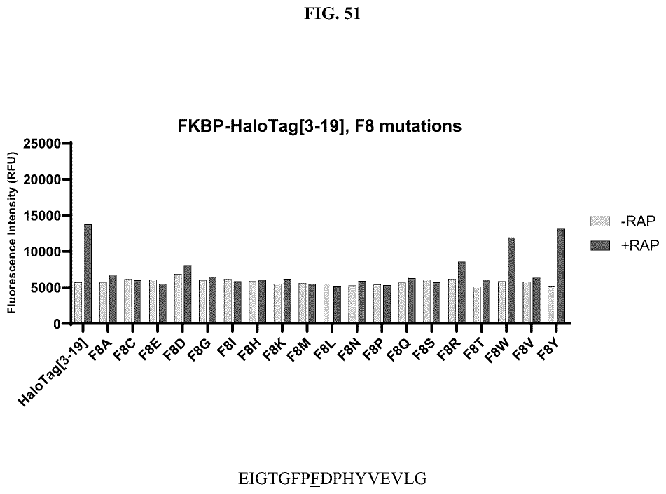

Mutation of residue F8 of the HaloTag[3-19] sequence showed, similar to F6, that this is a highly sensitive residue to mutation (FIG. 51). Only mutations F8W and F8Y were well tolerated, indicating that preserving the large hydrophobic sidechain at this position is needed for complementation. Some slight complementation activity was detectable with F8D and F8R, but these were lower than the unmutated HaloTag[3-19].

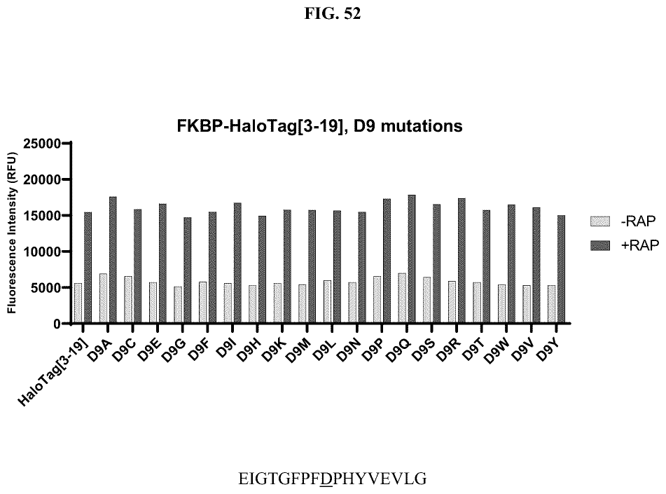

Mutation of residue D9 of the HaloTag[3-19] sequence showed that all mutations were well tolerated with some beneficial mutations coming from D9A, D9P, D9Q, and D9R (FIG. 52).

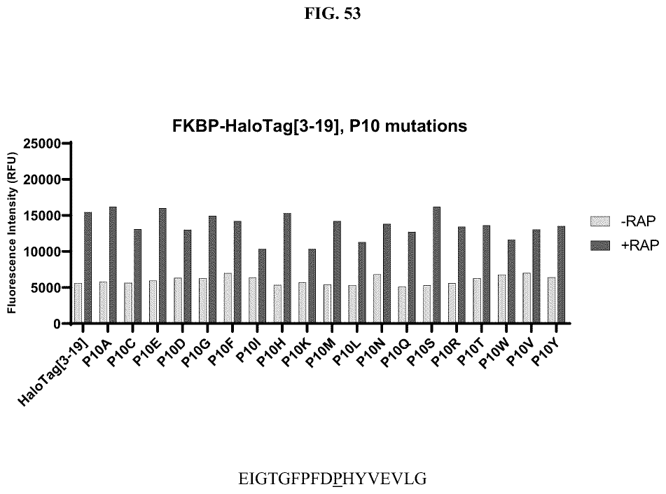

Mutation of residue P10 of the HaloTag[3-19] sequence showed moderate sensitivity to many mutations (FIG. 53). Mutations P10A, P10E, P10S, and P10H were among the most well tolerated. Mutations P10I and P10K were the most detrimental, although still functional. It should be noted that, similar to residue P7, mutations that are well tolerated at P10 as single mutations are mostly detrimental when combined with other mutations in HaloTag[3-19]. So, while mutations at P7 and P10 can be tolerated, they seem to be in their own category of positions that do not combine well with other mutations.

Mutation of residue H11 of the HaloTag[3-19] sequence showed improvement through H11N but H11D, H11G, and H11P were shown to be highly detrimental mutations (FIG. 54). Similar to F6, F8, and Y12, this is part of a set of residues that makes contact with the large fragment through their hydrophobic ring sidechains, although H11 seems to tolerate more mutations than the other three residues in the set.

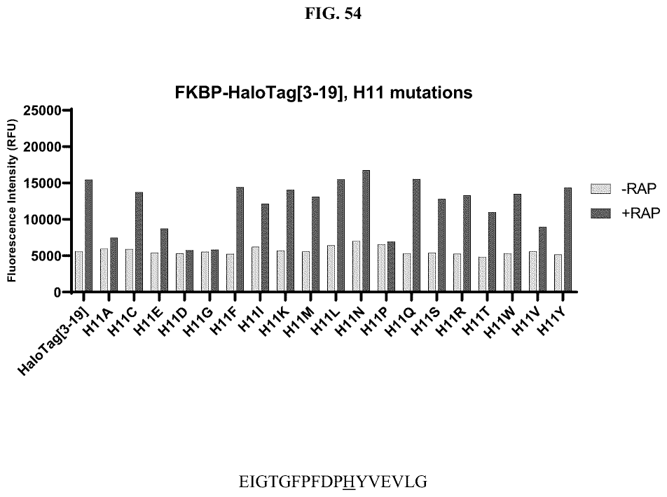

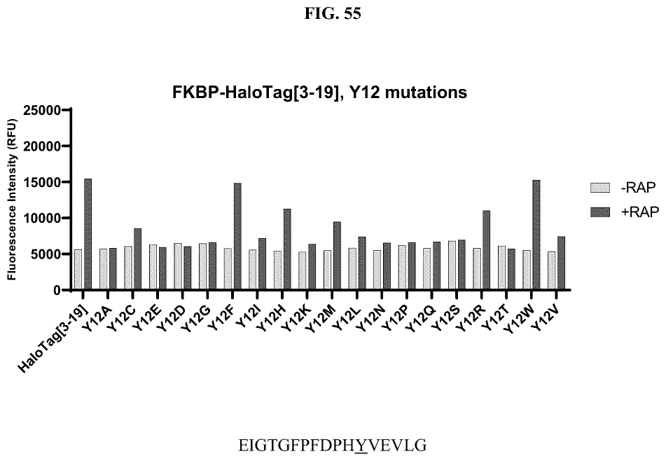

Mutation of residue Y12 of the HaloTag[3-19] sequence showed that it is highly sensitive to mutation, with tolerance for conservative mutations Y12F and Y12W that preserve its hydrophobic ring sidechain (FIG. 55). Some charged residues were tolerated as well but with lower activity, such as Y12H and Y12R. All other mutations seem to be very detrimental at this position. Along with F6, F8, and Y12, this is a large hydrophobic sidechain that only tolerates a few amino acid changes that preserve its characteristics.

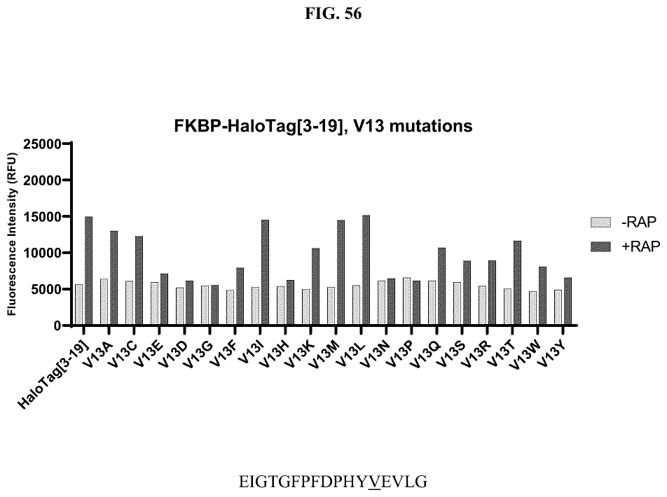

Mutation of residue V13 of the HaloTag[3-19] sequence showed a tolerance for other similar hydrophobic amino acids such as V13A, V13L, and V13I (FIG. 56). There were lower but active mutations with charged residues as well, with some notable non-functional mutations as shown.

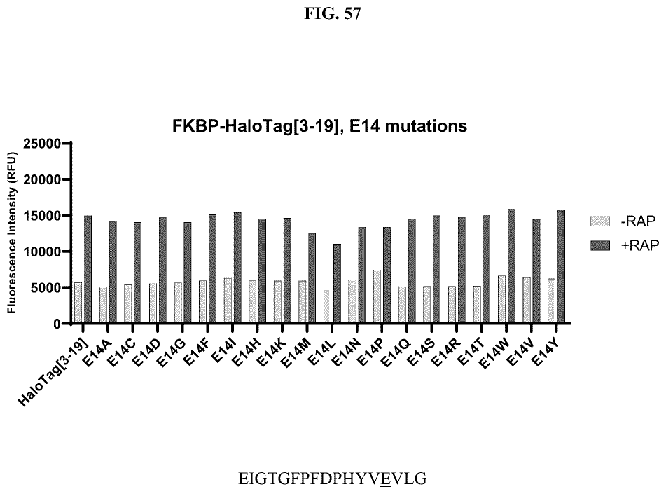

Mutation of residue E14 of the HaloTag[3-19] sequence showed tolerance across all mutations, with the possible exception of E14L that was lower than the unmutated sequence (FIG. 57).

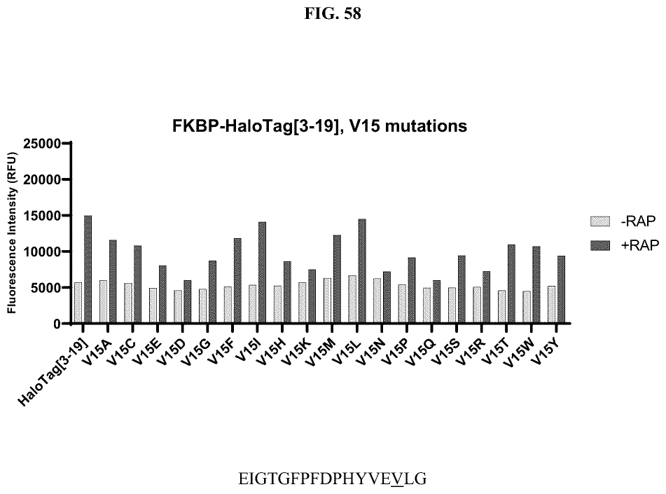

Mutation of residue V15 of the HaloTag[3-19] sequence showed tolerance to other hydrophobic amino acids, notably V15I and V15L that are conservative mutations (FIG. 58). It did not seem to tolerate charged residues well although many still had detectable activity.

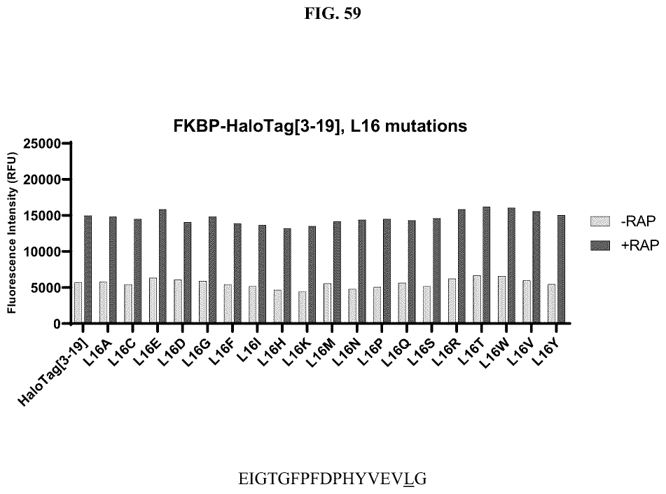

Mutation of residue L16 of the HaloTag[3-19] sequence showed tolerance to changes to all amino acids (FIG. 59).

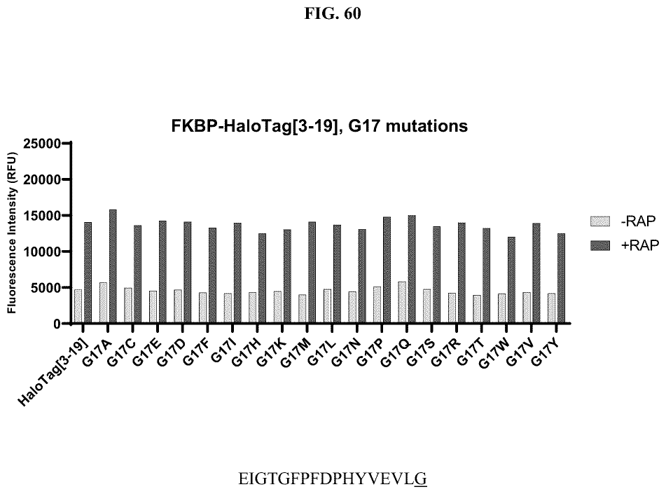

Mutation of residue G17 of the HaloTag[3-19] sequence showed tolerance to all amino acids, with a small preference for the G17A or G17Q mutations that have higher −RAP and +RAP intensities (FIG. 60).

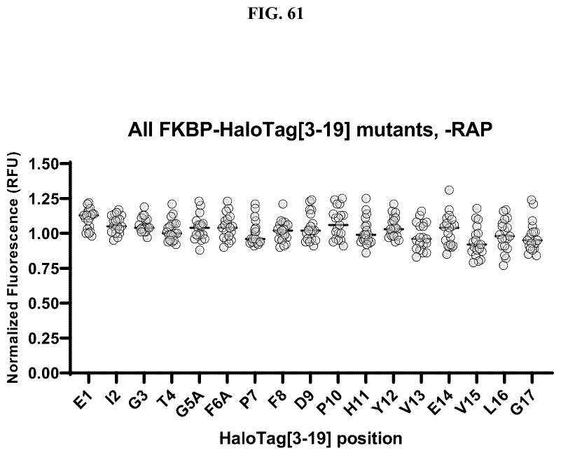

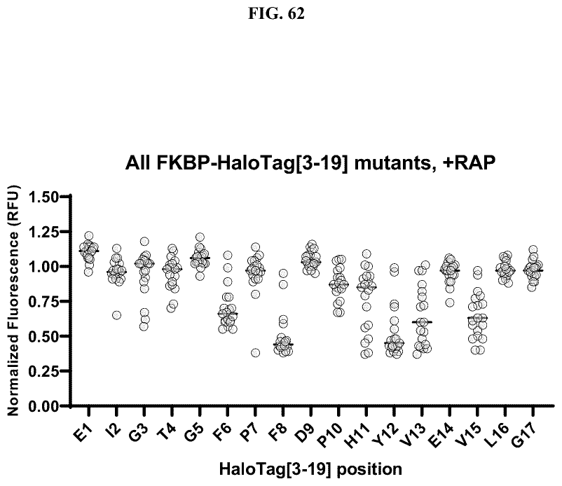

Aggregating all single amino acid changes into a single graph demonstrates that, in the absence of Rapamycin, there is some variation in the non-facilitated complementation of the HaloTag[3-19] mutants, with potentially a few mutants, e.g., F8D and E14P, that separate themselves with higher spontaneous interaction with the larger fragment (FIG. 61). Aggregation of the data on all single mutants in the presence of Rapamycin shows that some positions are highly tolerant of mutations, such as E1, G5, D9, E14, L16, and G17, for example (FIG. 62). There is a set of mutations that has low tolerance to mutations, with only a few conservative mutations that preserve the characteristics of the sidechains, such as F6, F8, H11, Y12, V13, and V15.

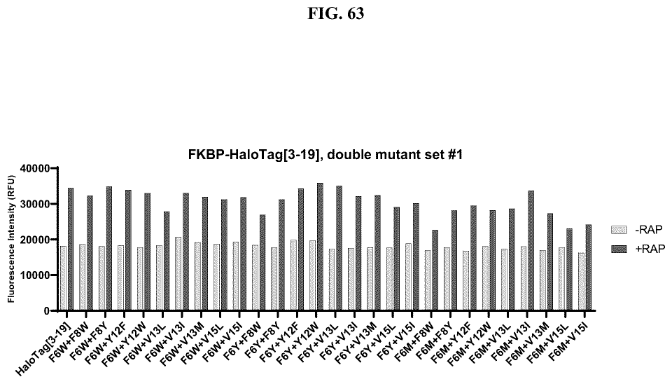

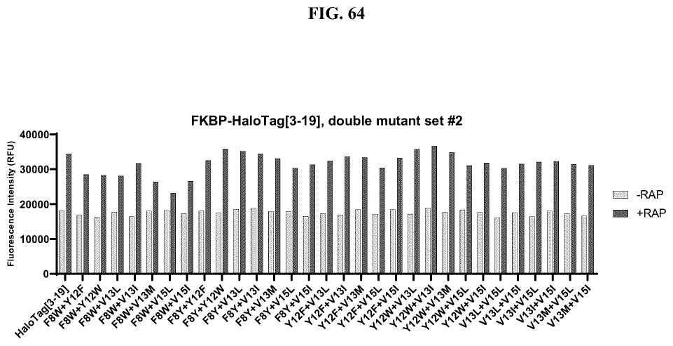

Based on single mutant data that showed positions F6, F8, H11, Y12, V13, and V15 were limiting in their tolerance to mutations, panels of double mutations were designed to determine how much diversity could be introduced starting from these stringent positions. Many combinations of mutations were tested at these positions (FIG. 63). Combinations with F6Y and F6W tended to be the best overall, with F6M combinations being tolerated, but showing lower activity. F8W also tended to be preferred across combinations than F8Y. This experiment showed that although these positions tolerate less diversity, they can still be mutated in combination to obtain a functional split HaloTag system. Preferred combinations included those with Y12W and F8Y. Mutations with V8W or V15L tended to be lower in activity (FIG. 64).

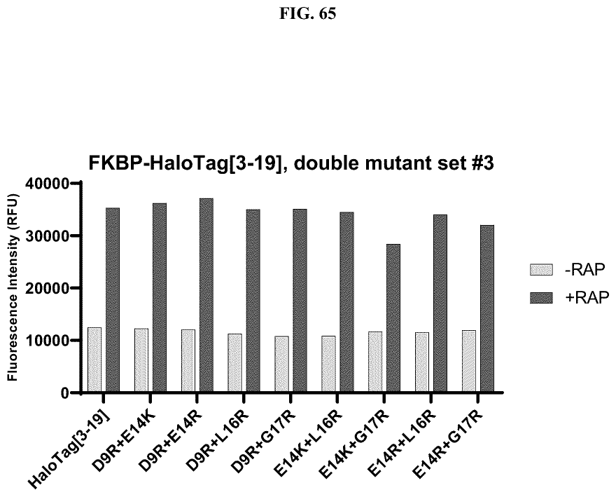

Double mutants were generated to target the highly tolerant positions in the HaloTag[3-19] fragment to determine if charged residues can be introduced in combination (FIG. 65). Multiple charge mutations can be introduced simultaneously, changing the characteristics of the sequence to highly negative or highly positively charged.

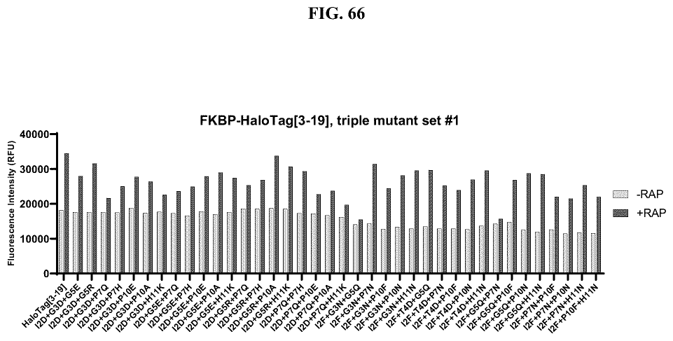

Triple mutant combinations showed that mutation combinations that incorporate changes at P7 or P10 tended to be much lower activity, although there are some preferred combinations that showed high activity, such as I2F+G3N+P7N and I2D+G5R+P10A (FIG. 66). These combinations introduced charged residues and mutated hydrophobic positions simultaneously, and many of them were well tolerated.

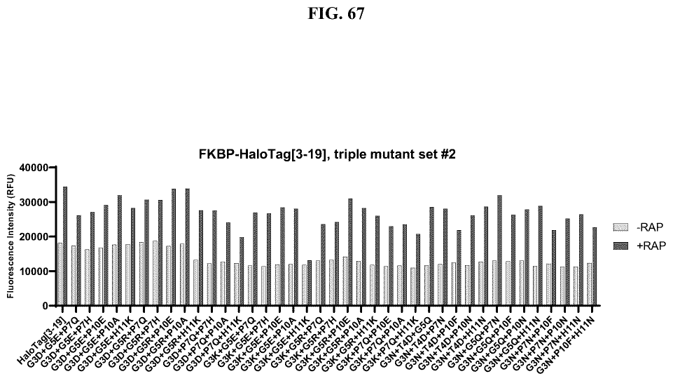

Combinations were generated to test the introduction of charged residues and mutating hydrophobic residues simultaneously (FIG. 67). Similar to other experiments, mutations to P7 or P10 tended to be less tolerated across all combinations. Many examples of introducing both charge and change of hydrophobic residues in the same sequence are shown, such as G3D+G5R+P10E that shows similar activity to the unmutated HaloTag[3-19].

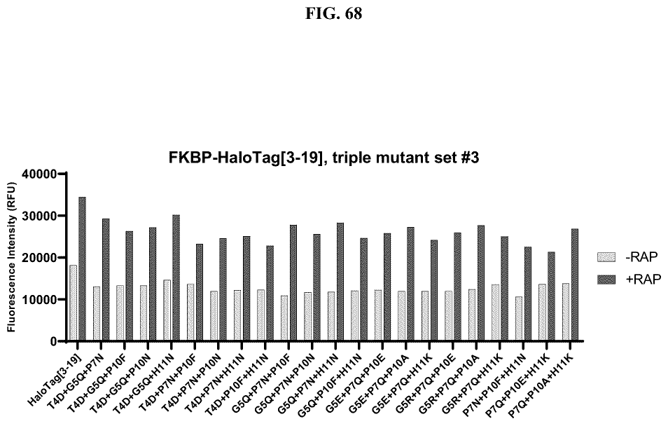

Combinations generated to test combinations where both the stringent P7 and P10 residues are mutated together show good activity, such as G5R+P7Q+P10A (FIG. 68).

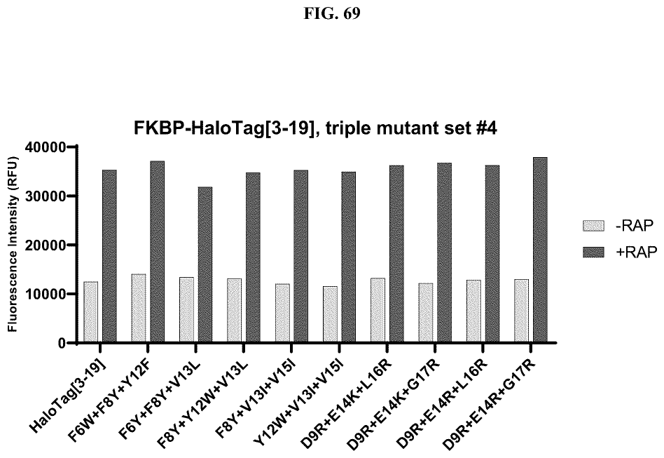

Triple mutations generated including combinations at three of the stringent hydrophobic residues F6, F8, and Y12 show that if tolerated mutations are selected at each position all of them can be changed in a single combination, such as F6W+F8Y+Y12F (FIG. 69). More charges can also be introduced, such as several arginine residues, e.g., D9R+E14R+G17R.

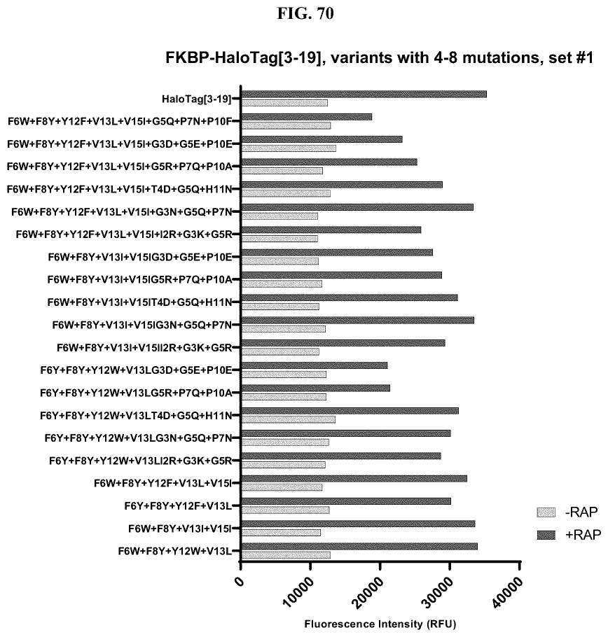

Expanding on the double and triple mutant combinations that were well tolerated, mutations continued to be combined, showing that many positions can be mutated simultaneously in the HaloTag[3-19] sequence, including those that change all the stringent hydrophobic residues and proline residues together, such as F6W+F8Y+Y12F+V13L+V15I+G3N+G5Q+P7N, a sequence with 8 of the 17 positions mutated (FIG. 70).

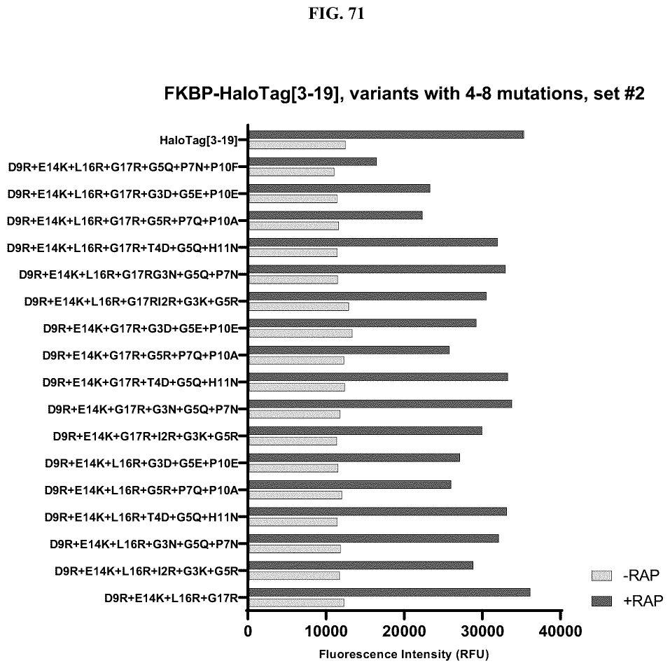

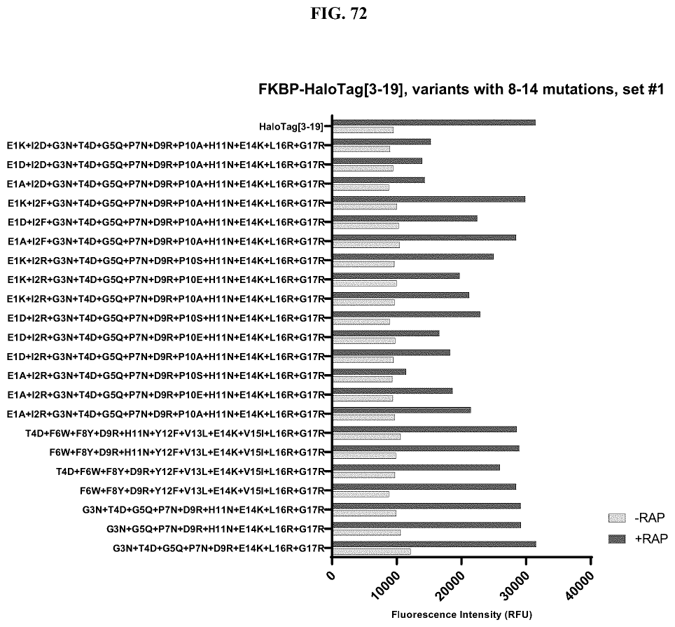

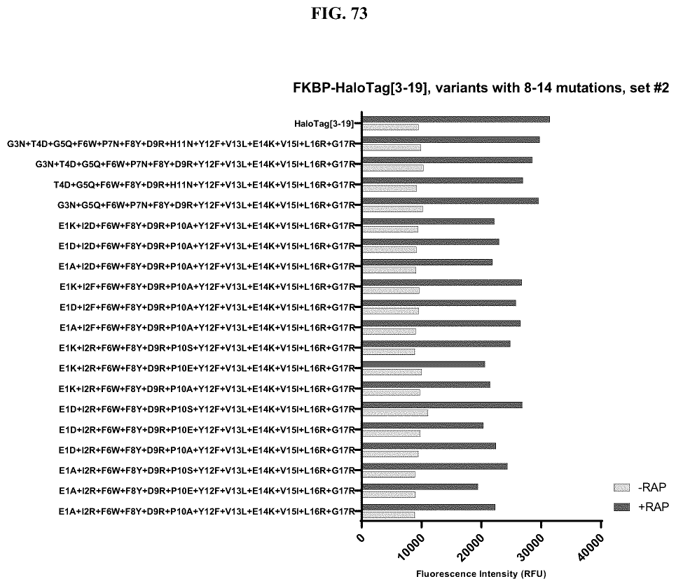

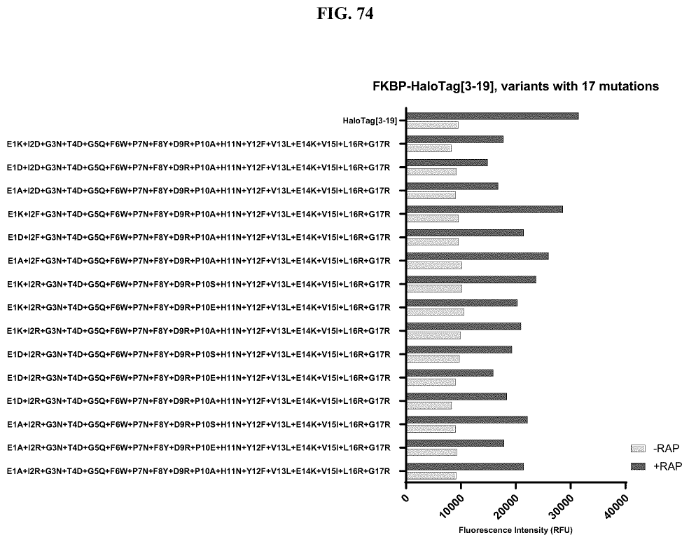

A multiple mutation set was generated focusing on introducing many charged residues into the sequence and mutating the stringent proline residues together (FIG. 71). Many combinations of the P7 and P10 mutated together tended to be detrimental, although most are still functional. Mutagenesis was further expanded to show that by using many of the well tolerated mutations at more positions, HaloTag[3-19] sequences with up to 12 positions mutated can be generated (FIG. 72). Additional combinations identified a top mutant with 14 positions mutated that shows activity similar to the unmutated HaloTag[3-19] control (FIG. 73). Mutants were tested in which all 17 positions in the HaloTag[3-19] were mutated simultaneously (FIG. 74). This demonstrated several sequences still showing complementation activity. Notably, E1K+I2F+G3N+T4D+G5Q+F6W+P7N+F8Y+D9R+P10A+H11N+Y12F+V13L+E14K+V15I+L16R+G17R showed similar activity to the unmutated HaloTag[3-19] with all 17 positions mutated in the sequence (0% identity to HaloTag[3-19]). This example shows that side chain characteristics (hydrophobicity, charge, etc.) rather than identity are sufficient for providing the interactions with the large fragment in complementation assays.

### Example 7

### Mutagenesis of the HaloTag[22-297](M2F) or “Large HaloTag” Fragment

Experiments were conducted during development of embodiments herein to explore the impact of single and multiple substitutions on the expression and/or activity of the HaloTag[22-297](M2F) fragment. The expression and activity data of the HaloTag[22-297](M2F) variants is provided in Table 2.

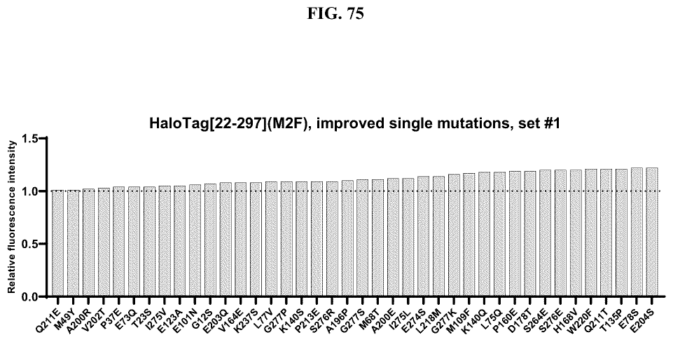

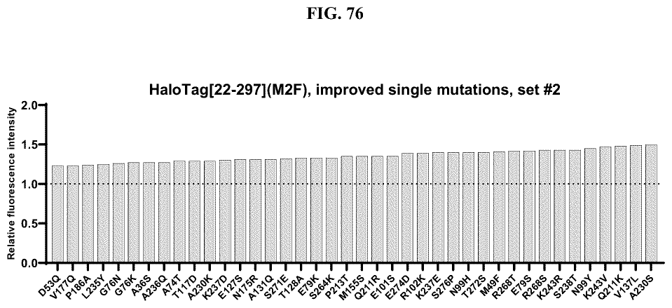

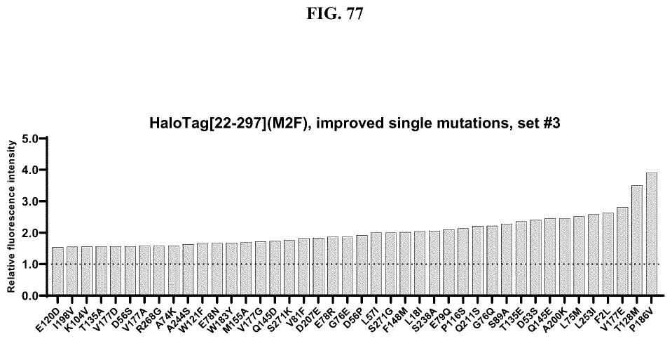

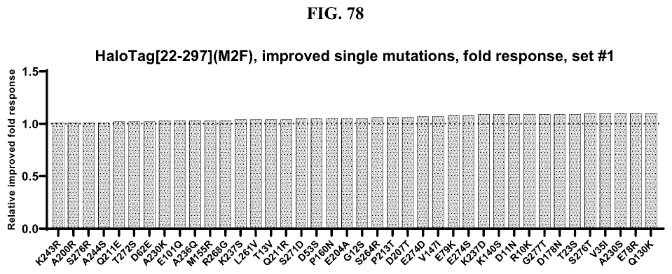

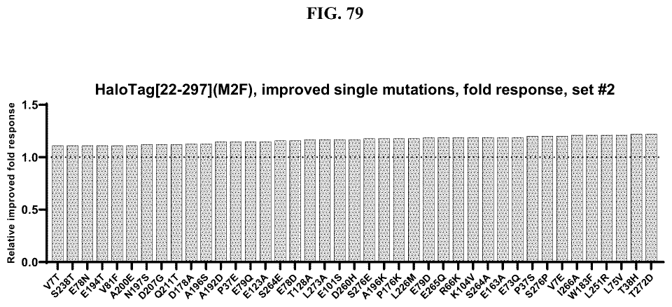

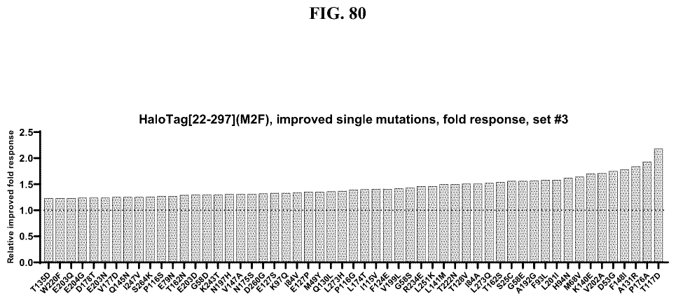

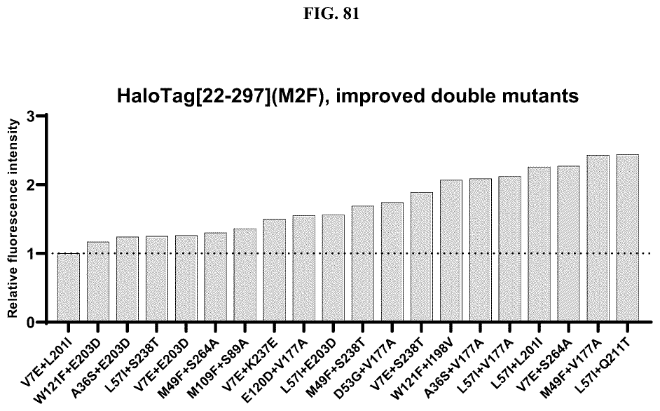

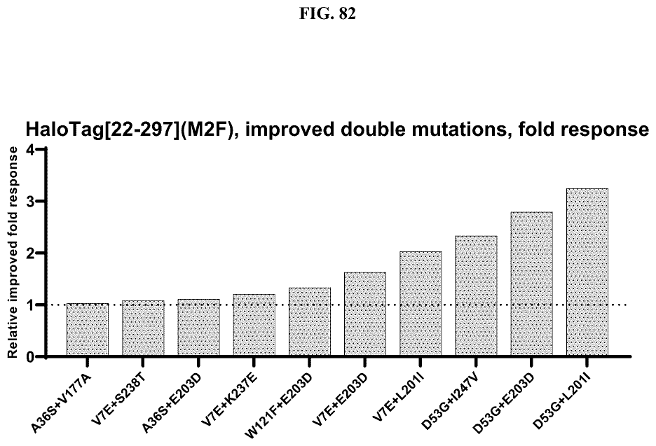

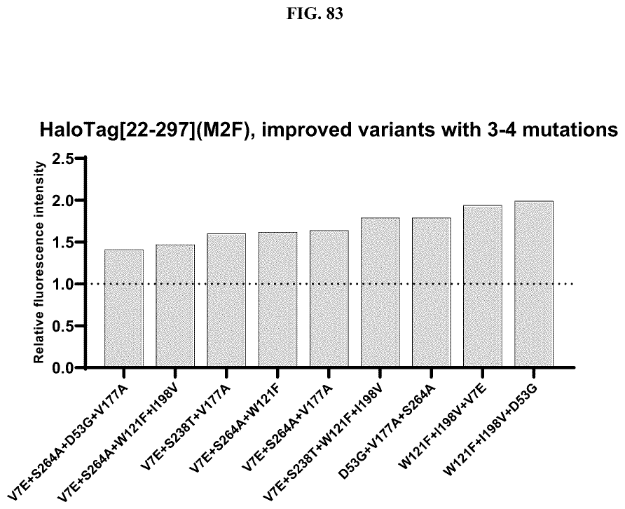

Single mutations were identified that improve the expression and/or activity of the HaloTag[22-297](M2F) fragment (FIGS. 75-77). Single mutations were identified that improve the fold response of the HaloTag[22-297](M2F) fragment (FIG. 78-80). Double mutations were then identified that improve the expression and/or activity (FIG. 81) or fold response (FIG. 82) of the HaloTag[22-297](M2F) fragment. Triple mutations were identified that improve the expression and/or activity of the HaloTag[22-297](M2F) fragment (FIG. 83).

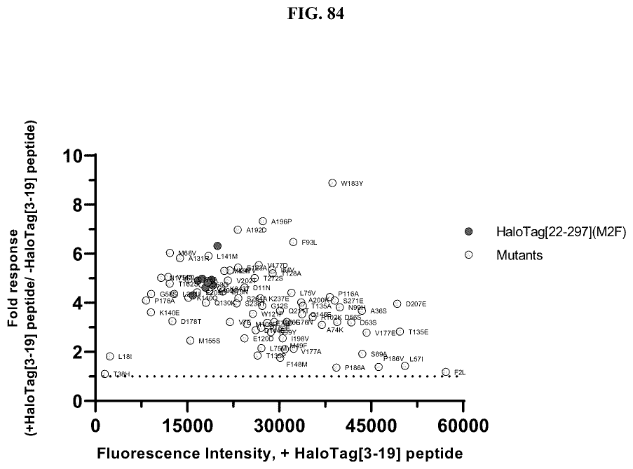

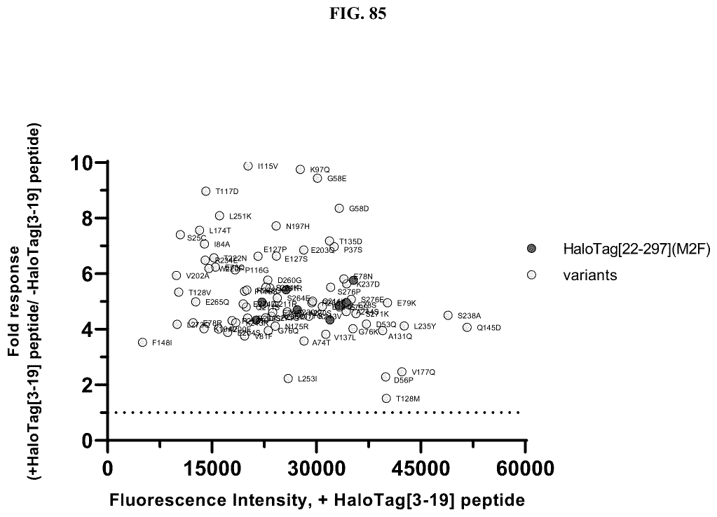

Experiments were conducted during development of embodiments herein to demonstrate that when excess peptide is present to facilitate maximum interaction with the best HaloTag[22-297](M2F) mutants, many have further improved activity relative to the unmutated control (FIGS. 84-85). There are several mutants with greater than 3-fold improved expression and/or fold response.

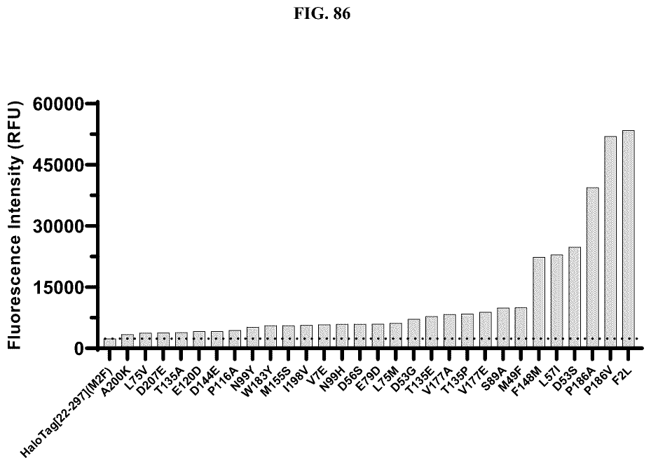

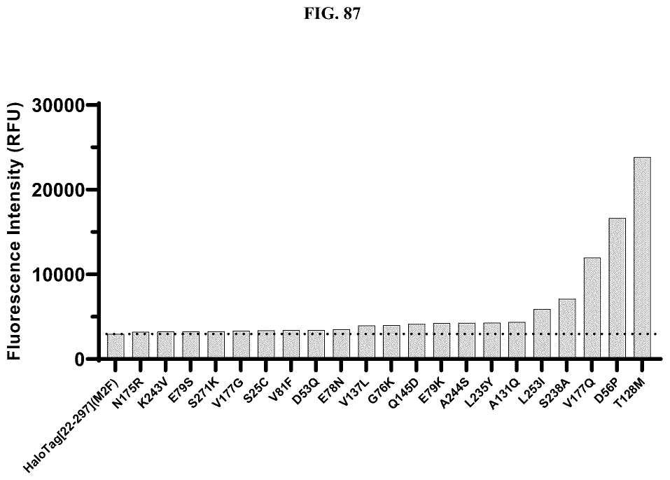

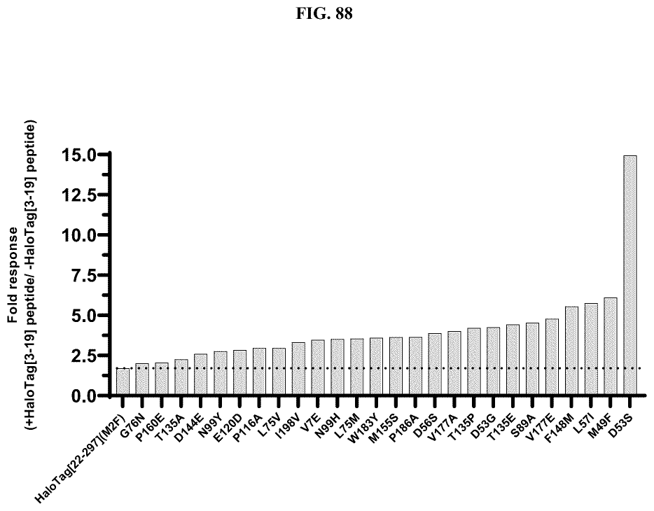

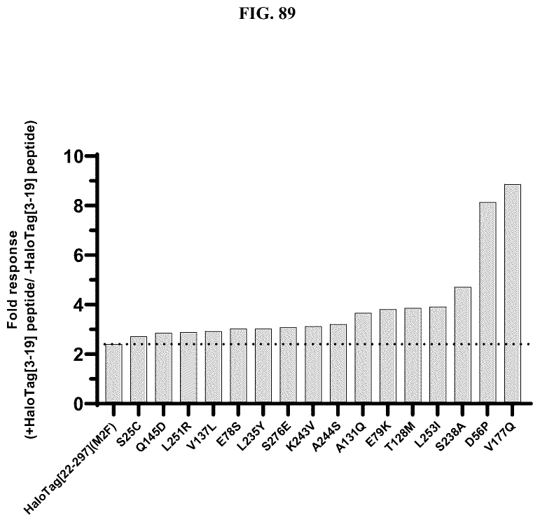

Thermal challenge conducted with complemented HaloTag[22-297](M2F) mutants shows many mutations that improve the expression and stability of the protein while retaining responsiveness to peptide (FIGS. 86-87). Thermal challenge of complemented HaloTag[22-297](M2F) mutants shows many mutations that improve the fold response of the protein where the complemented complex has higher thermal stability relative to the unmutated control (FIGS. 88-89).

### Example 8

### Split HaloTag Use in Mammalian Cells

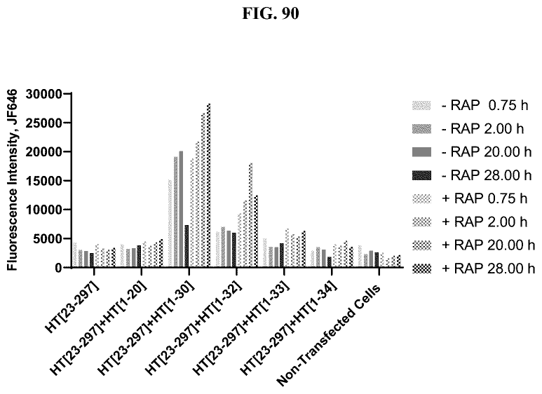

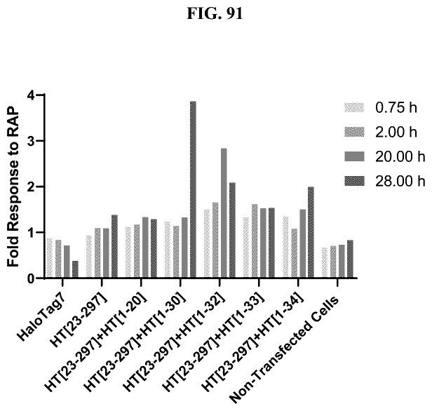

A plate-based fluorescent assay was conducted demonstrating that split HaloTag fragments can complement each other in the FRB/FKBP model system (FIG. 90). The best SmHT in this assay was HaloTag[1-30], but it also exhibited higher background (no Rapamycin) complementation. Fold response from the assay is depicted in FIG. 91).

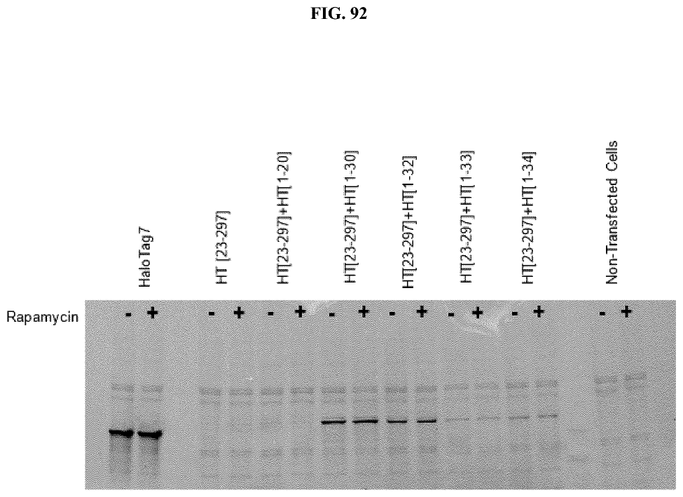

Gel-based assays were conducted to confirm that the LgHT fragment is the species being labeled with HaloTag ligand by showing the correct size band on a fluorescence gel (FIG. 92).

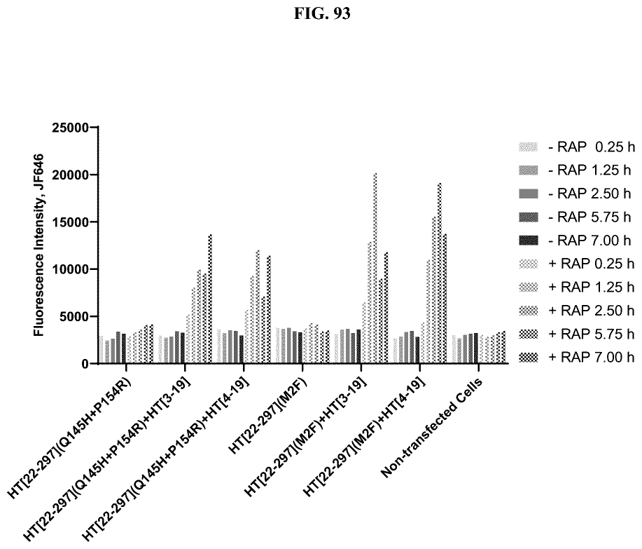

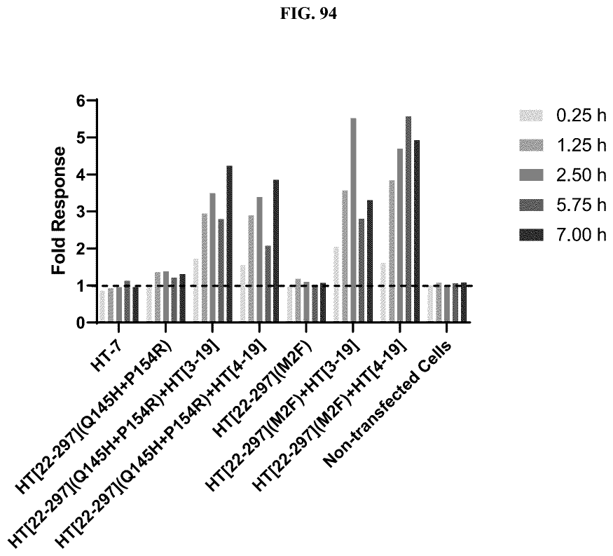

SmHT optimization was performed by truncation, demonstrating that HaloTag[3-19] and [4-19] showed good fluorescence intensities and high fold responses (FIG. 93). Both SmHT sequences work with the two LgHT sequences tested in the experiment. Fold response from the assay is depicted in FIG. 94).

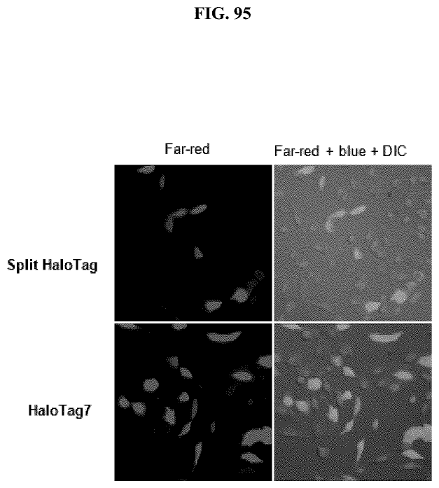

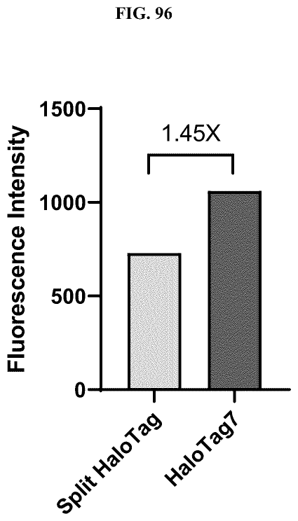

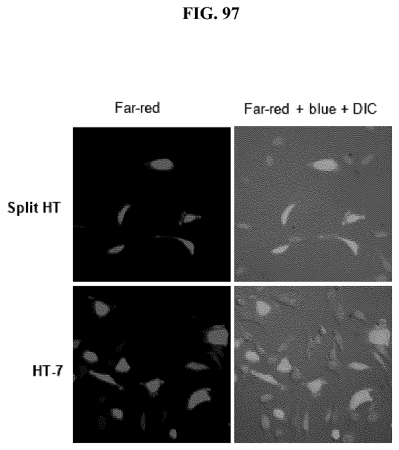

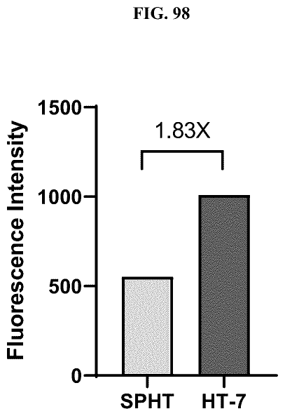

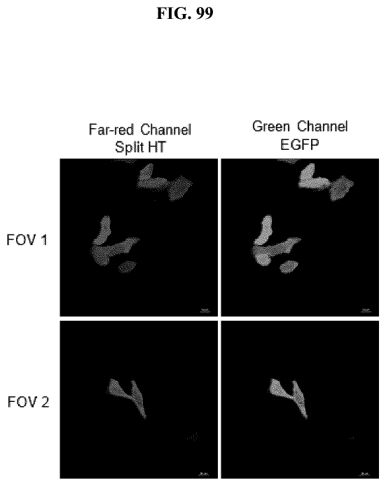

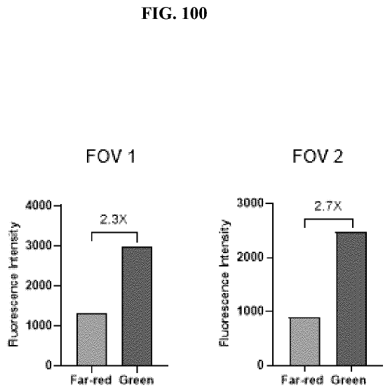

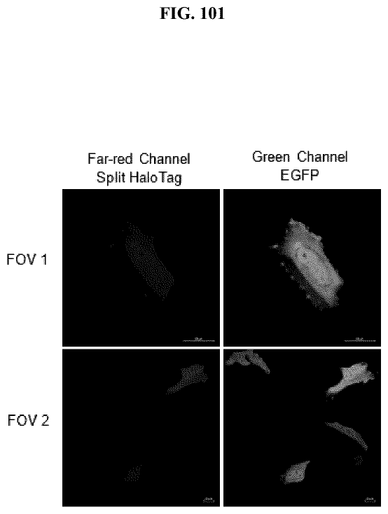

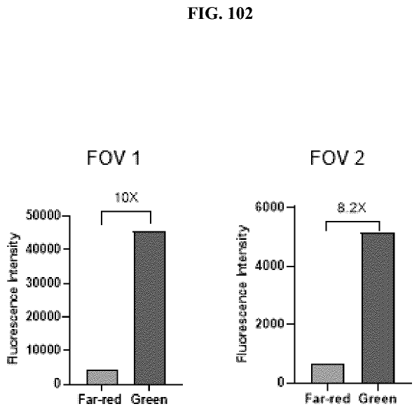

Experiments were conducted during development of embodiments herein to demonstrate that split HaloTag fragments can be used for detection of protein interactions by fluorescence microscopy (FIGS. 95-100). In the absence of Rapamycin, the split HaloTag fragments show very little labeling with JF646 ligand using microscopy (FIG. 101). Quantitation shows the difference in labeling in the two fluorescence channels. GFP signal is high because it tracks expression of the SmHT construct, but the Far-red is low since there is no Rapamycin to facilitate complementation and reconstitution of split HaloTag activity.

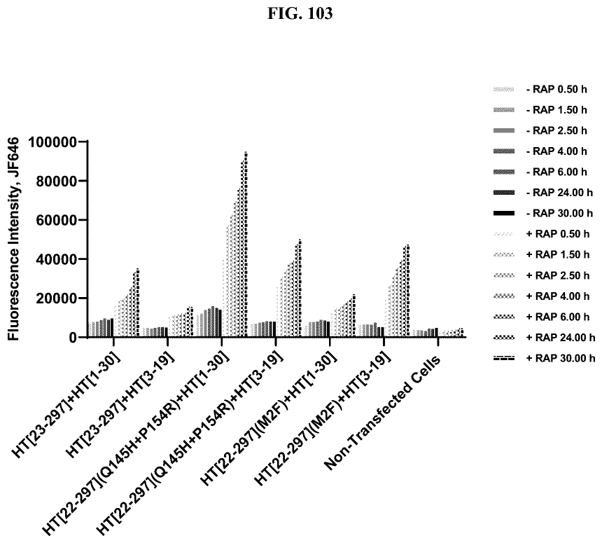

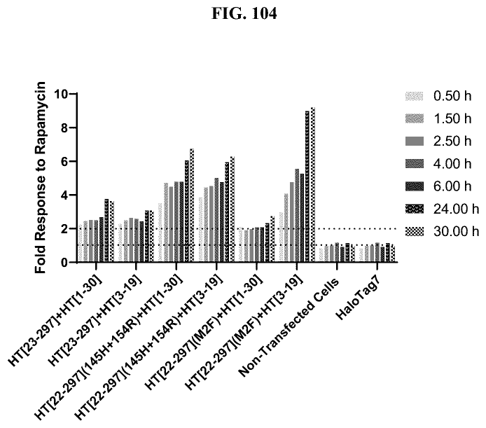

Comparison of LgHT and SmHT variants in mammalian cell assays show that many configurations comprising different sequences of both can be used to detect protein interactions (FIGS. 103-104).

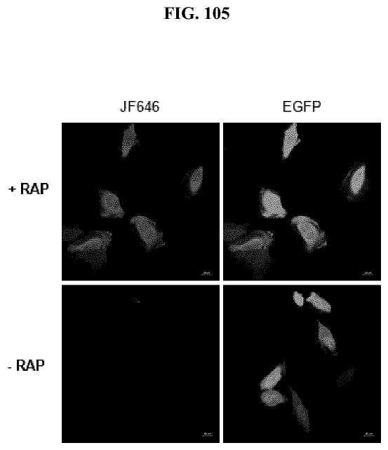

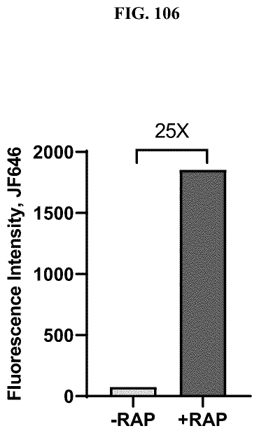

Experiments were conducted during development of embodiments herein using a more optimized system comprising the HaloTag[3-19] fusion and a variant LgHT fusion (FIGS. 105-106). This shows high specificity of the fluorescence labeling of the split HaloTag only in the presence of Rapamycin to facilitate the interaction of their fusion partners. A 25-fold response to Rapamycin was observed.

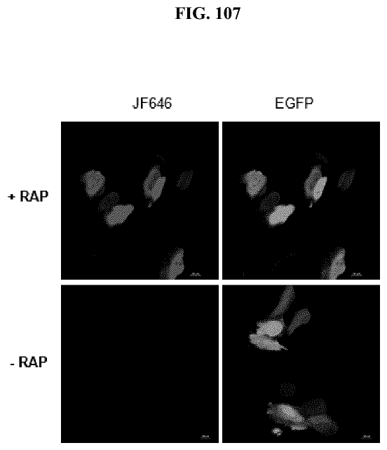

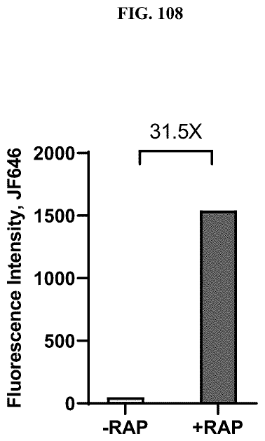

Experiments were conducted during development of embodiments herein using the HaloTag[3-19] fusion and the M2F variant of LgHT as a fusion (FIG. 107-108). High specificity of the fluorescence labeling of the split HaloTag was only observed in the presence of Rapamycin to facilitate the interaction of their fusion partners.

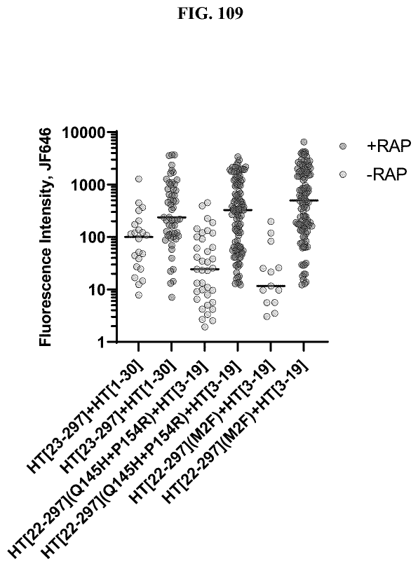

Experiments were conducted during development of embodiments herein to demonstrate quantitation of cell imaging data from several fields of view for different SmHT and LgHT variant combinations (FIG. 109). The median cell intensity increases in the presence of Rapamycin across all the configurations shown, with perhaps the best combination being the HaloTag[22-297](M2F)+HaloTag[3-19] variants.

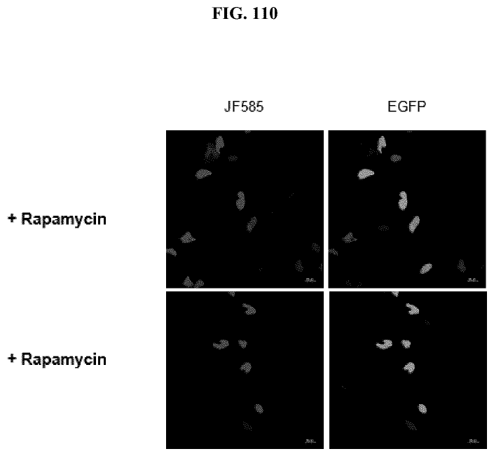

Experiments were conducted during development of embodiments herein extending the demonstrated use in cell imaging with more HaloTag ligands (FIG. 110). JF585 HaloTag ligand was used. EGFP shows the expression and localization of the SmHT fusion independent of labeling activity.

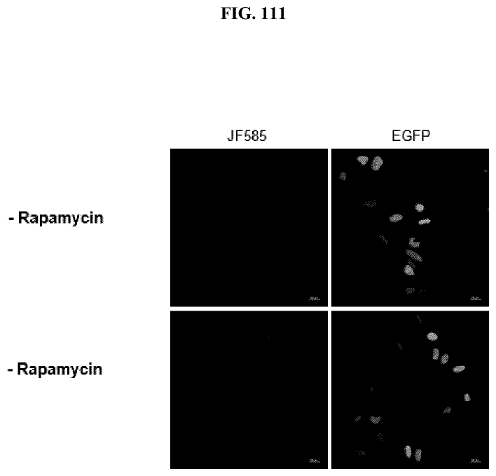

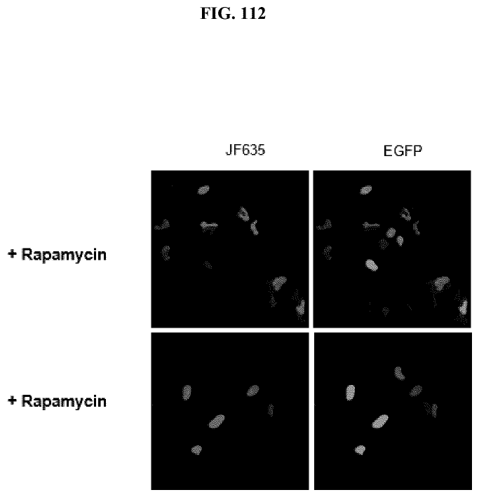

Live cell imaging was conducted of split HaloTag activity in mammalian cells using JF585 HaloTag ligand in the absence of facilitated interaction between split HaloTag fragments. Experiments demonstrated the background level of labeling using JF585 HaloTag ligand, where there is no facilitation in the interaction with Rapamycin (FIG. 111). In another demonstrated use in cell imaging with more HaloTag ligands, using JF635 HaloTag ligand, EGFP shows the expression and localization of the SmHT fusion independent of labeling activity (FIG. 112).

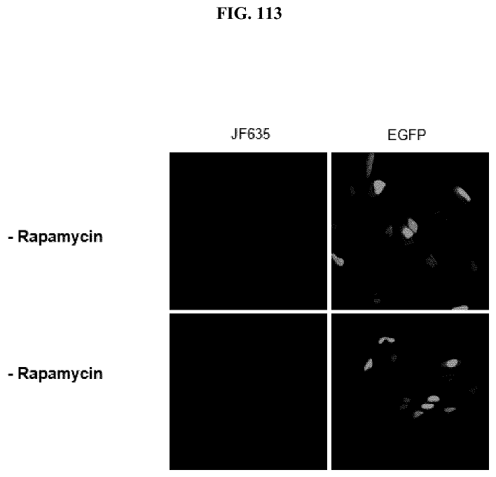

Live cell imaging was conducted of split HaloTag activity in mammalian cells using JF635 HaloTag ligand in the absence of facilitated interaction between split HaloTag fragments. Experiments demonstrated the background level of labeling using JF585 HaloTag ligand, where there is no facilitation in the interaction with Rapamycin (FIG. 113).

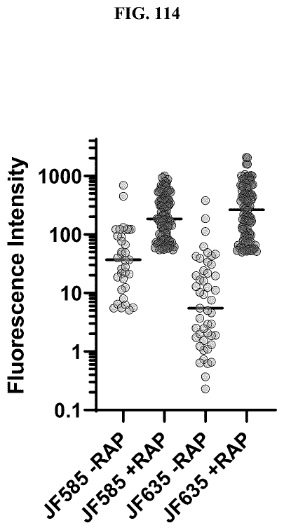

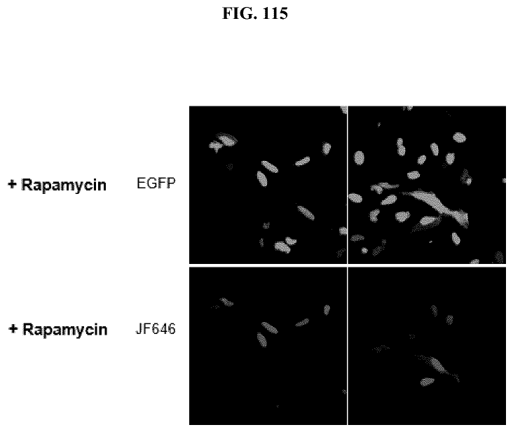

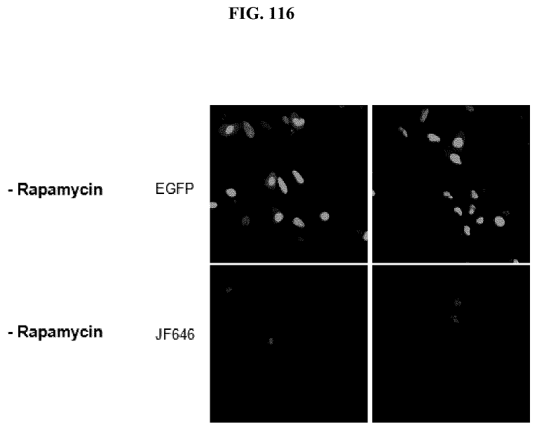

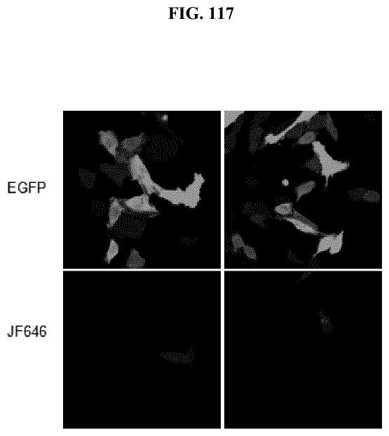

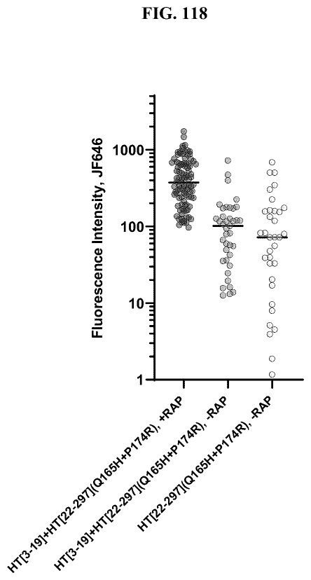

Experiments were conducted during development of embodiments herein to compare the fluorescent intensity of all imaged cells in serval fields of view in +/−RAP conditions with fluorogenic ligand JF585 and JF635 (FIG. 114). Quantitation of individually imaged cells demonstrates the use of JF585 and JF635 HaloTag ligands provided a Rapamycin-dependent increase in the median intensity of fluorescence of the imaged cells. The controls in the experiment combining the more optimal HaloTag[3-19] fragment variant with the HaloTag[22-297](Q145H+P154R), showing that in the absence of Rapamycin, there is very low labeling of the split HaloTag. In other words, this shows the low background of the system (FIG. 116). Another control in the experiment combining the more optimal HaloTag[3-19] fragment variant with the HaloTag[22-297](Q145H+P154R) shows that in the absence of the HaloTag[3-19] fragment, there is very low labeling of the large HaloTag fragment. A quantitative difference in fluorescence intensity of cells is observed when compared to controls lacking Rapamycin or the HaloTag[3-19] fragment.

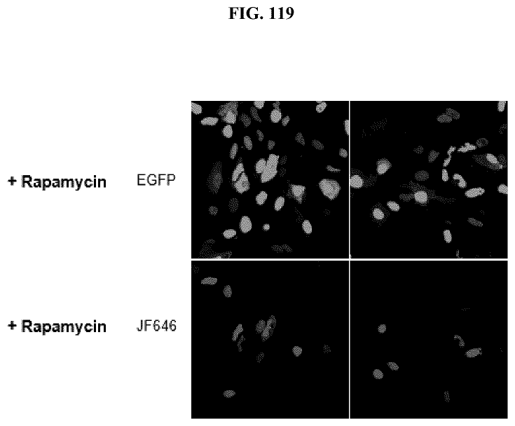

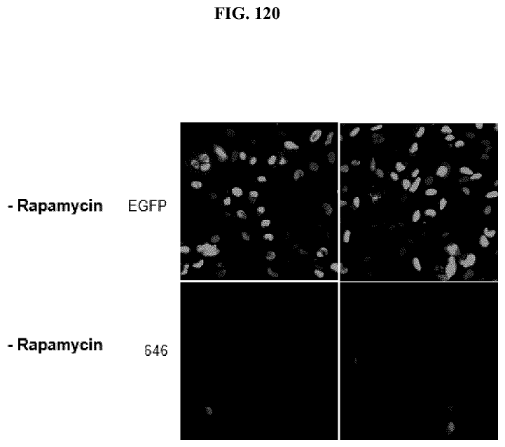

Experiments were conducted during development of embodiments herein using the combination of the more optimal HaloTag[3-19] fragment variant with the HaloTag[22-297](M2F) variant and measuring complementation in the model FRB/FKBP system with live cell fluorescent imaging (FIG. 119). In the absence of Rapamycin, there is very low labeling of the split HaloTag. In other words, this shows the low background of the system (FIG. 120).

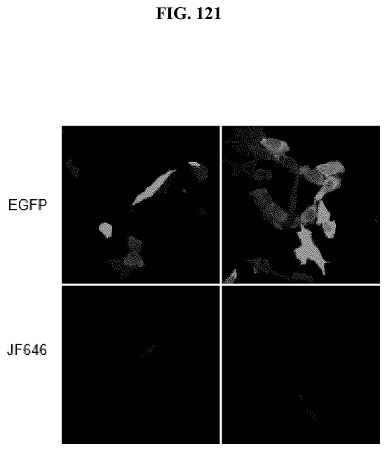

Experiments were conducted during development of embodiments herein combining a more optimal HaloTag[3-19] fragment variant with the HaloTag[22-297](M2F), showing that in the absence of the HaloTag[3-19] fragment, there is very low labeling of the large HaloTag fragment (FIG. 121). The low background of the system is demonstrated. In agreement with the live mammalian cells plate assay experiments, the S:B ratio in +rapamycin/−rapamycin conditions is higher with HaloTag[22-297](M2F), about 6.7, compared to HaloTag[22-297](Q145H+P154R), about 4. For both of the large HaloTag fragments, the background signal from the self-complementation without Rapamycin is higher than the background from labeling the Large HaloTag in the absence of the HaloTag[3-19] fragment. The signal-to-background ratio in the presence of Rapamycin for complemented split HaloTag over labeling the large HaloTag alone is 16 and 5.7 for HaloTag[22-297](M2F) and HaloTag[22-297](Q145H+P154R), respectively.

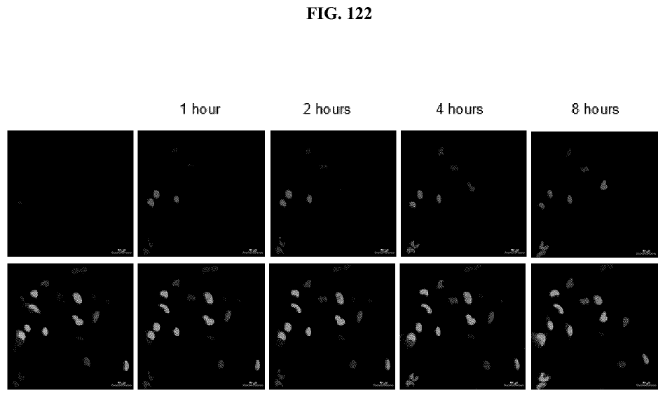

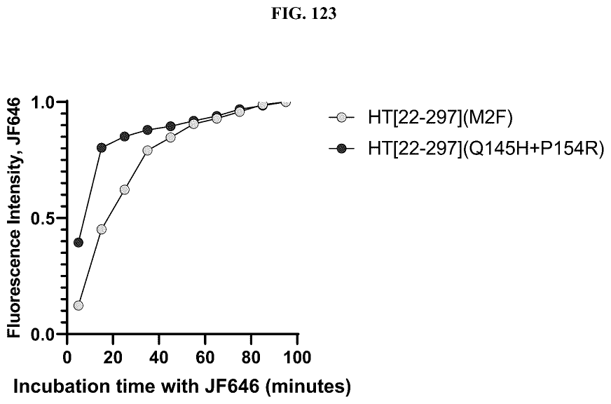

Kinetic Studies were conducted using complementation and labeling kinetics of the split HaloTag (FIGS. 122-123). To measure the complementation and labeling rate of the split HaloTag in the FRB/FKBP model system, a time-lapse imaging experiment was used. Transfected cells were immediately imaged every 15 minutes for 12 hours after the addition of both Rapamycin and JF646 HaloTag ligand. These experiments showed that even 1 hour incubation with Rapamycin is sufficient time to image cells with high expression of the constructs.

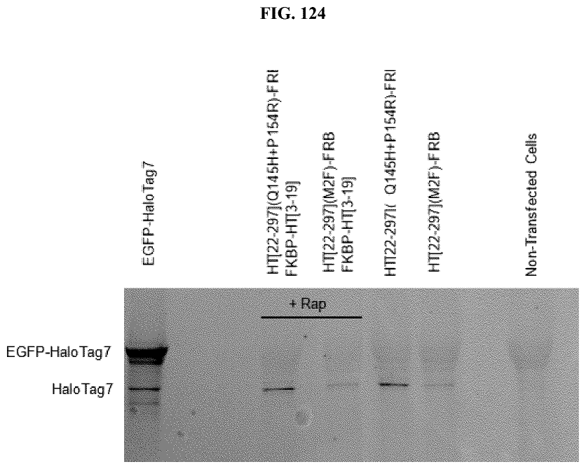

Experiments were conducted during development of embodiments herein to compare the expression of HaloTag[22-297](Q145H+P154R) vs. HaloTag[22-297](M2F) as complemented with the small HaloTag fragment HaloTag[3-19] and non-complemented form in mammalian cells (FIG. 124). The lytic TMR assay demonstrated that HaloTag[22-297](Q145H+P154R) expressed better than HaloTag[22-297](M2F) in mammalian cells as both complemented with the small HaloTag fragment and non-complemented forms.

### Example 9

### Detection of Protein:Protein Interactions (BRD4:Histone H3 Interaction)

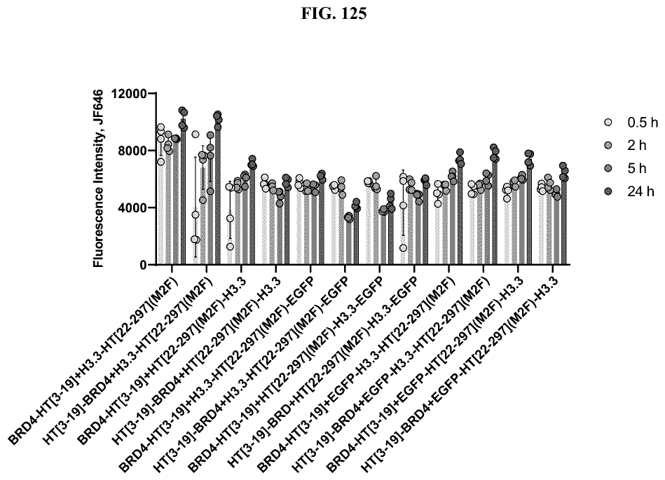

The BRD4:Histone H3.3 is a constitutive protein:protein interaction (PPI) in mammalian cells (no inducer is necessary). Fusion of the split HaloTag fragments as indicated allowed for detection of the interaction by labeling with JF646 in plate-based assays (FIG. 125).

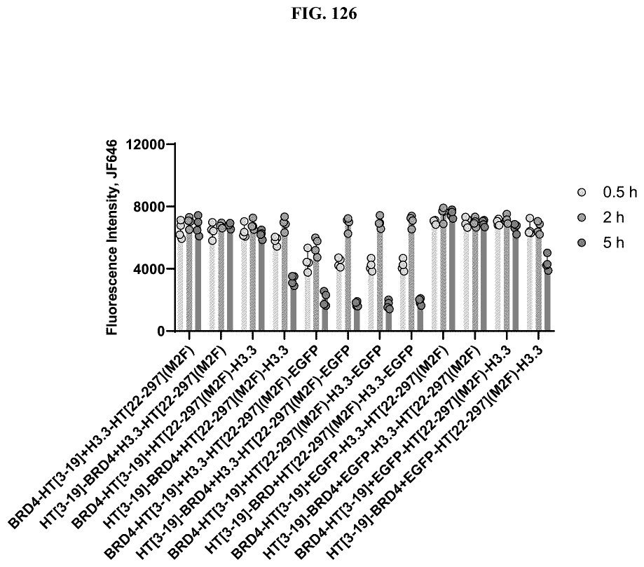

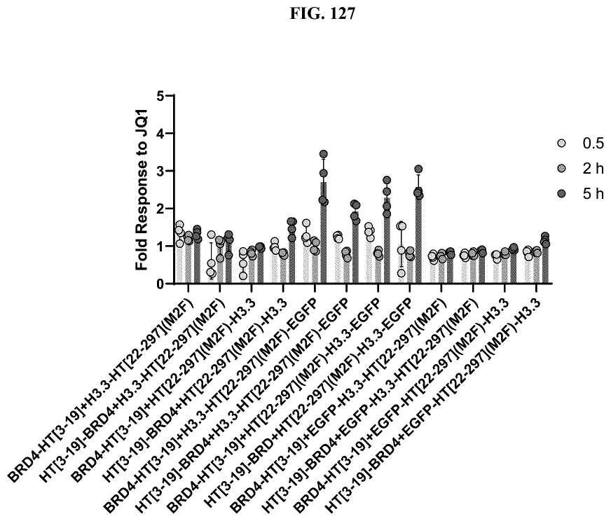

Reversibility of a PPI with split HaloTag can be measured by inhibiting previously assembled protein complexes in cells using drug compounds (FIG. 126). Fold response of BRD4:Histone H3 interaction to JQ1 inhibitor shows that, in several configurations of the split HaloTag fragments, the inhibition of the interaction between BRD4 and Histone H3 can be detected (FIG. 127).

In addition to plate-based assays, split HaloTag can be used to detect protein:protein interaction in live cells using fluorescence microscopy (FIG. 128).

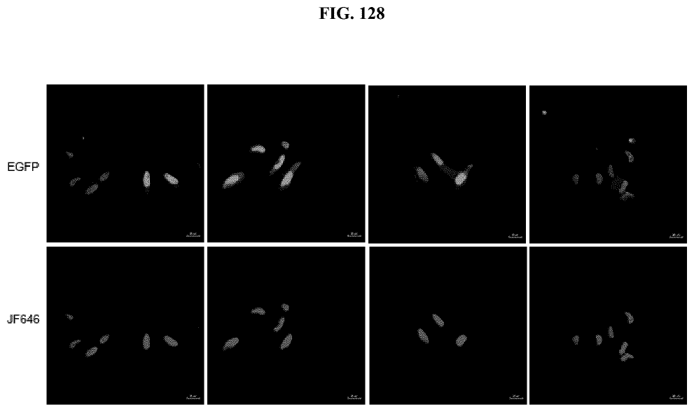

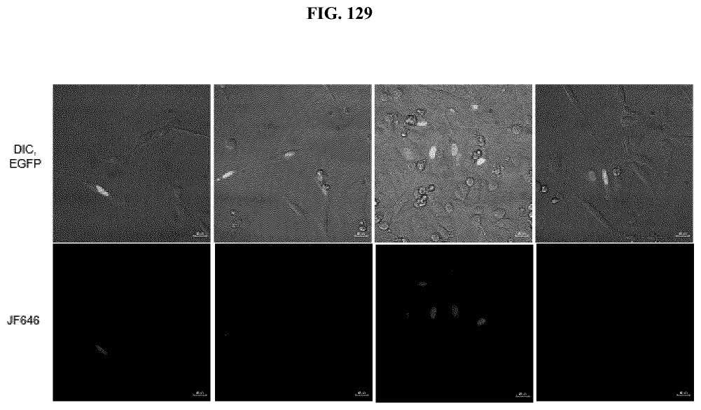

Inhibition of the BRD4:Histone H3 interaction can be detected with fluorescence microscopy as well since treatment of cells with the JQ1 interaction inhibitor significantly reduces the labeling with JF646 HaloTag ligand as the split HaloTag fragments no longer complement efficiently (FIG. 129).

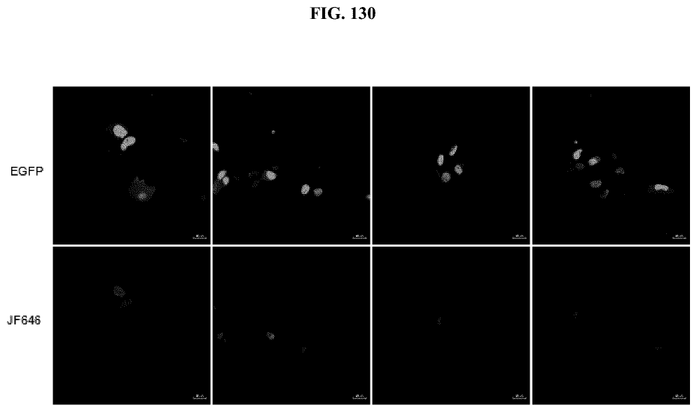

In the absence of the HaloTag[3-19] fragment, very little labeling with JF646 HaloTag ligand was observed, demonstrating the specificity of the labeling for the presence of the complementing split HaloTag fragments (FIG. 130).

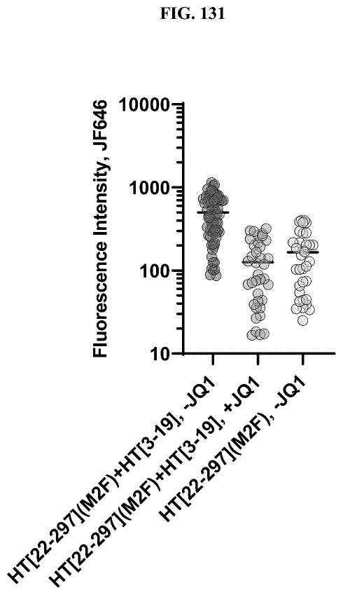

Quantitation of cells imaged across experiments shows the measurable decrease in median intensity of JF646 HaloTag ligand labeling when treated with an inhibitor of the interaction (FIG. 131).

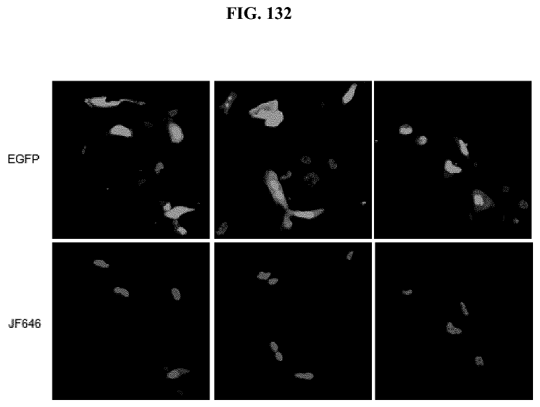

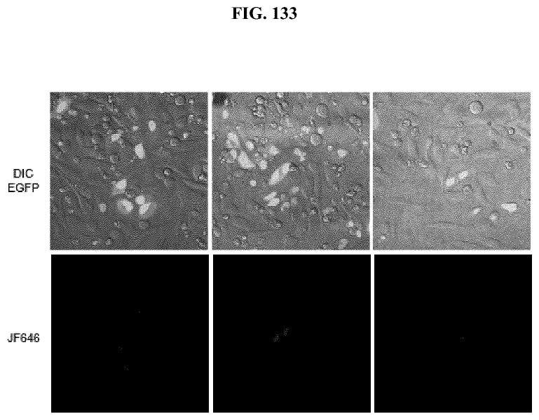

A second set of imaging experiments was performed to confirm reproducibility and show improvement of the system's signal-to-background ratio by changing a few confocal microscope settings, such as lower gain and laser intensity (FIG. 132). Lower treatment with JQ1 inhibitor still showed nearly complete lack of labeling with JF646 HaloTag ligand, indicating measurement of the lack of interaction between the protein fusion targets (FIG. 133).

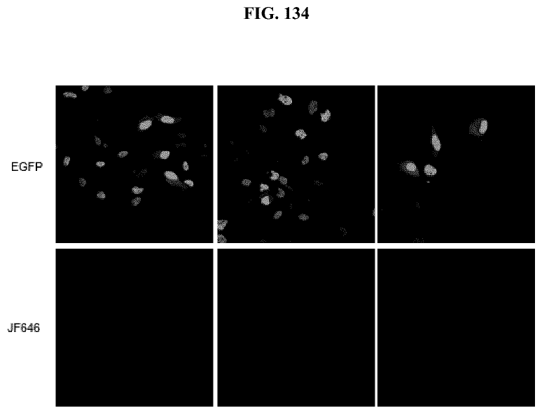

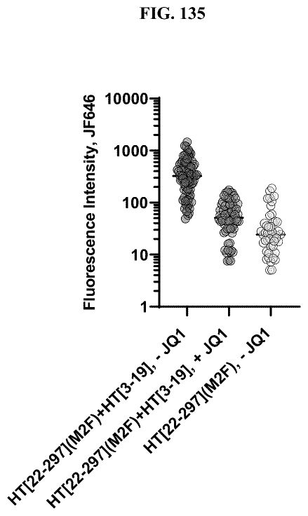

In the first imaging experiment (FIG. 127-130), images were collected with 85 far-red laser intensity resulting in S:B ratio of about 4. The purpose of the second experiment (FIG. 131-134) with lower signal intensity was to see if most of the background signal could be eliminated with the cost of the overall reduction in the specific signal in the far-red channel. Quantifying the imaging data showed that lower laser intensity reduced the background more than the specific signal, leading to enhanced S:B ratios 6 and 12 for background in the presence of JQ1 and background from labeling the large HaloTag, respectively (FIG. 135).

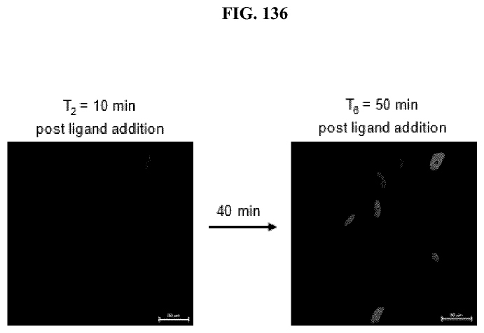

Experiments were conducted during development of embodiments herein to measure the JF646 HaloTag ligand labeling kinetics of the pre-existing complemented complex in live cells (FIG. 136). Since the reaction of BRD4 and histone H3.3 does not require an inducer, the binding complex starts to form as the two fusions are expressed in cells. Therefore, this system is a good candidate to measure the rate of complemented split HaloTag constructs labeling with JF646 ligand. Notably, labeling consists of making the covalent bond with the chloroalkane moiety and activating the fluorescence, which cannot be measured separately by imaging.

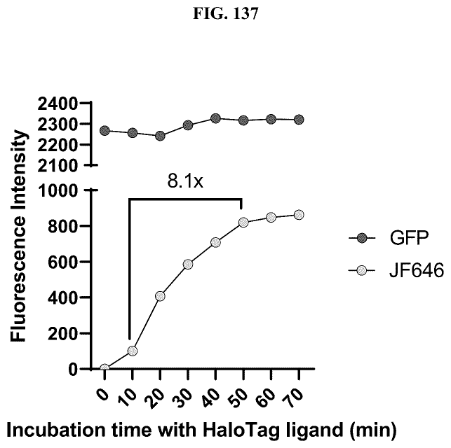

To measure the labeling rate, cells were immediately imaged after ligand addition every 10 minutes for 70 minutes (FIG. 137). A Z-stack image was obtained at all time points to ensure all cells were captured in focus. The most focused Z levels were merged into one, and the intensity of all cells (6 total objects) was measured and averaged at all time points. Thus, every dot on the plot for both the far-red and green channels is the average of the cell intensities in the imaged field of view. Quantifying the data shows that the signal in the far-red channel reaches its maximum in about 40 minutes, and as expected, the intensity in the green channel remains unchanged during the measurement.

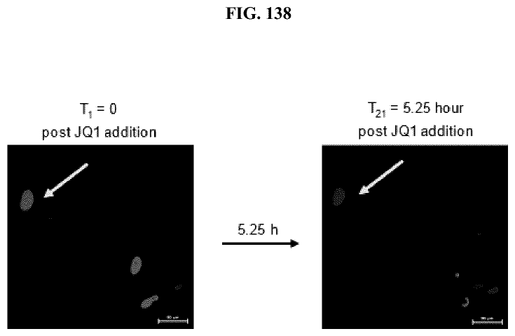

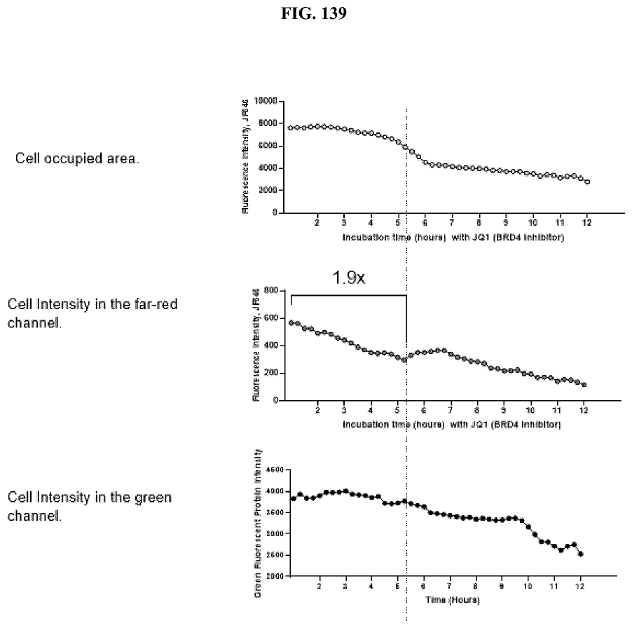

Experiments were conducted during development of embodiments herein to determine the effect of SmHaloTag peptide dissociation on the fluorescence intensity (FIG. 138). Measurement of the fluorescence intensity of Split HaloTag after the dissociation of the Small HaloTag peptide, HaloTag[3-19], was tested. After the separation of the SmHaloTag, the Large HaloTag, HaloTag[22-297](M2F), is still covalently bound to the ligand but does the Small HaloTag dissociation affect the ligand fluorogenicity/activity? Time-lapse live cell imaging was performed to address this question. To ensure the tracked cell was viable during the experiment, the area occupied by that specific cell was measured at all time points. The results of the cell area show that that cell was stable and lived up to about 5 hours, but then after that it started to shrink. During this period, the cell intensity reduces almost by half indicating that although the Large HaloTag, HaloTag[22-297](M2F), is bound with ligand but JF646 is losing its fluorogenicity/activity slowly upon SmHaloTag dissociation.

### Example 10

### Detection of Protein:Protein Interactions (Calmodulin:M13 Interaction)

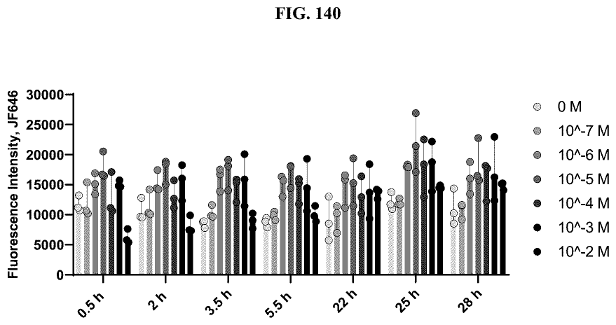

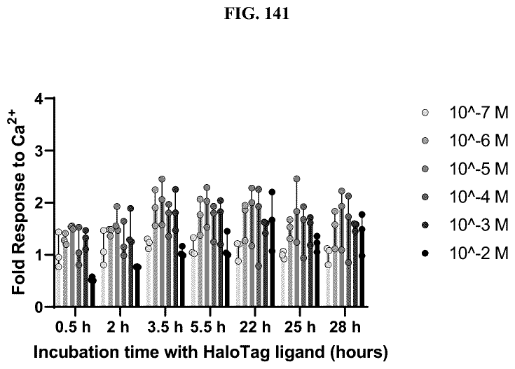

Similar to studying other protein-protein interactions, the fluorescent activity of all different fusion orientations was measured (FIG. 140) and fold response was calculated (FIG. 141). The C-terminal fusion of the small HaloTag fragment in complementation with M13-HaloTag[22-297](M2F)-EGFP resulted in the highest activity. A modest, calcium-dependent increase in fluorescence of the split HaloTag when fused to Calmodulin and M13 peptide was observed, where the presence of calcium facilitates the interaction between the proteins.

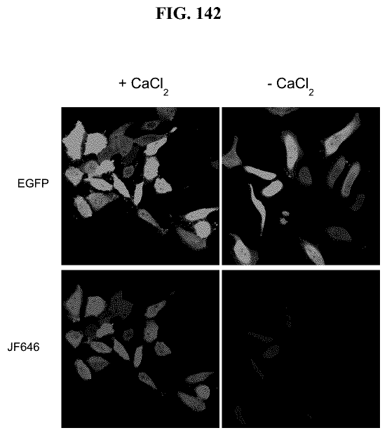

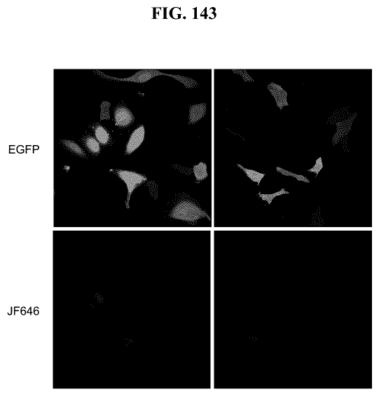

The use of split HaloTag for measuring the interaction between Calmodulin and the M13 peptide in live mammalian cells using fluorescence microscopy was demonstrated (FIG. 142). The addition of calcium chloride to induce the interaction caused a significant increase in fluorescence from the split HaloTag complementation. There is negligible background of the HaloTag[22-297](M13) fragment alone without the presence of the HaloTag[3-19] fragment in live cells (FIG. 143), indicating the signal is specific to the complementation of the fragments facilitated by the Calmodulin:M13 interaction.

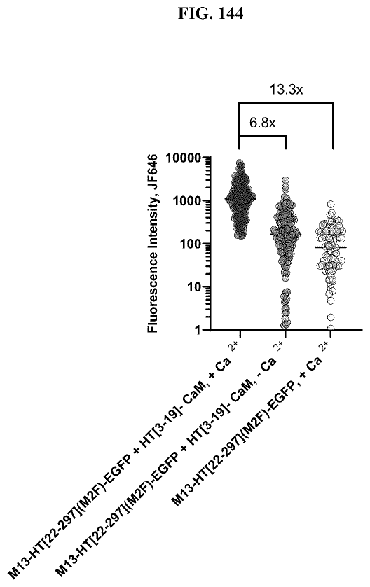

Quantitation of the split HaloTag imaging data for this model system indicates that a 7× increase in median fluorescence was observed across all cell images in the presence of calcium that facilitates the interaction (FIG. 144). The expression of the LgHT alone has low background activity and does not contribute significantly to the specific signal observed.

### Example 11

### Split HaloTag to Detect PROTAC Ternary Complex Formation

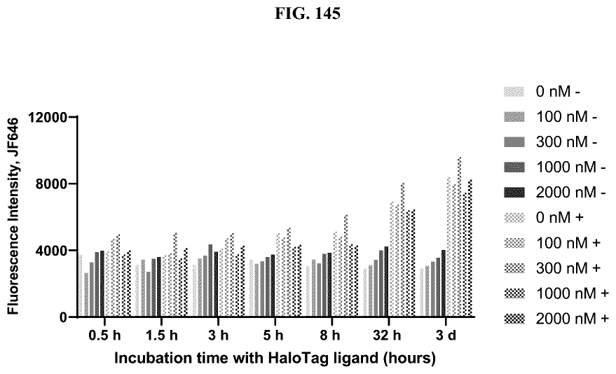

Experiments were designed to determine if the split HaloTag can be applied as a detector for tracking targeted protein degradation (FIG. 145). As a first step, the formation of a ternary complex (PROTAC ligand+E3 ligase+Target protein) by fusing split HaloTag fragments to the E3 ligase and Target protein to observe JF646 HaloTag ligand labeling dependence on PROTAC addition to cells to facilitate the interaction was measured. Additionally, imaging was done to determine the possibility of imaging of the PROTAC ternary complex using split HaloTag, which would have valuable information about its kinetics and localization as it forms in live mammalian cells. Similar to studying other protein-protein interactions, the fluorescent activity of all different fusion orientations was measured. Testing the activity of all different fusions to split HaloTag fragments helped to see a trend of better activity if the protein of interest (POI) is fused to the C-terminus of the small HaloTag fragment. Large HaloTag fragments, on the other hand, showed less sensitivity.

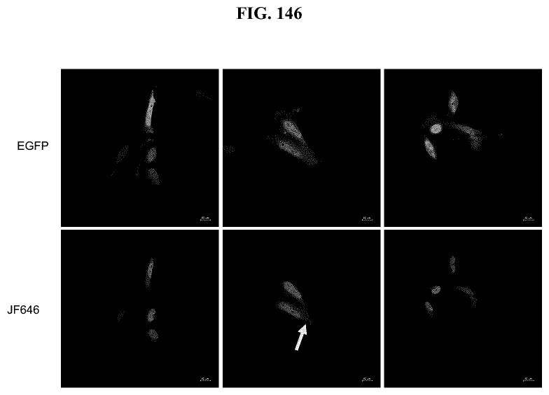

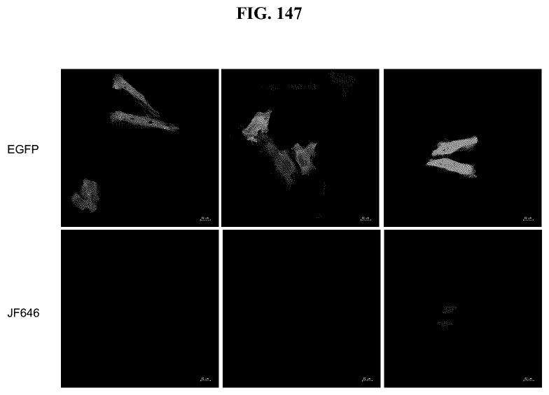

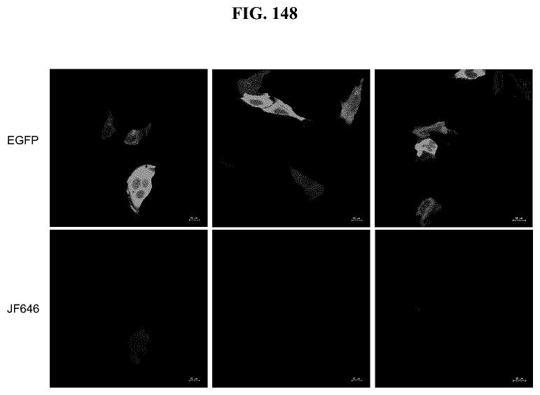

Imaging of the ternary complex formation in live mammalian cells provides information on subcellular localization of the complex using fluorescence microscopy (FIG. 146), depends on the addition of the PROTAC ligand, since little or no signal is observed in its absence (FIG. 147), and depends on the presence of the HaloTag[3-19] fragment, since the LgHT fragment alone has a very low signal (FIG. 148).

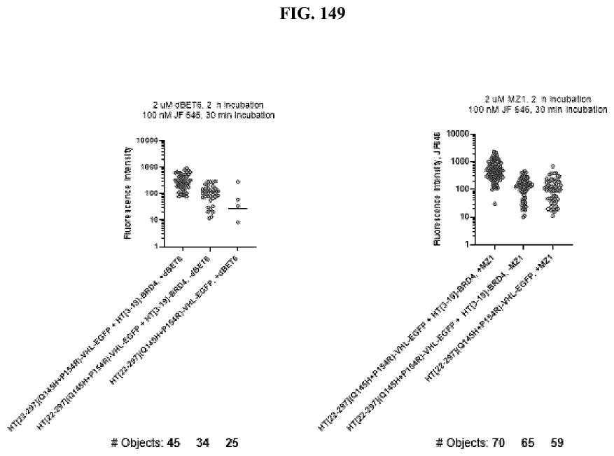

A PROTAC-dependent increase in JF646 HaloTag ligand fluorescence was observed due to complementation of split HaloTag when fused to the E3 ligase and target protein in the assay, demonstrating that detection of the ternary complex formation (FIG. 149). The signal-to-background ratio in +/−PROTAC ligand is higher with VHL compared to CRBN E3 ligase, 6 and 3, respectively.

### Example 12

### Split HaloTag to Detect Protein:Protein Interactions and PROTAC Ternary Complex Formation after CRISPR Introduction of the SmHT-HiBiT Tag onto the Chromosome (Targeting an Endogenous Protein, BRD4)

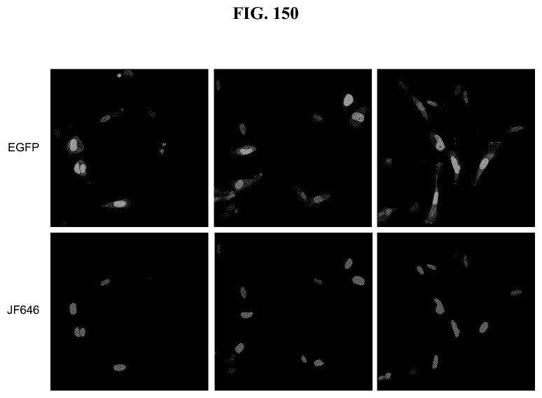

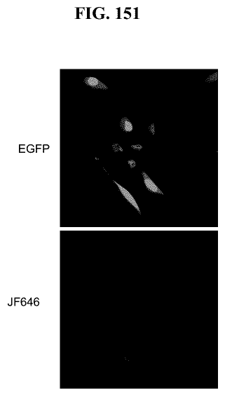

Experiments were conducted during development of embodiments herein to demonstrate the detection of a protein:protein interaction where the HaloTag[3-19] fragment has been introduced into the genome using CRISPR genome editing (FIG. 150). Here, it was fused as a dual tag (SmHT-HiBiT) to BRD4, and then LgHT-Histone H3 fusion on a plasmid introduced to demonstrate detection of the interaction with fluorescence imaging after JF646 HaloTag ligand addition. As a control, in the absence of the CRISPR edit to introduce the dual tag, little or no fluorescence labeling of the LgHT expressed alone was observed (FIG. 151).

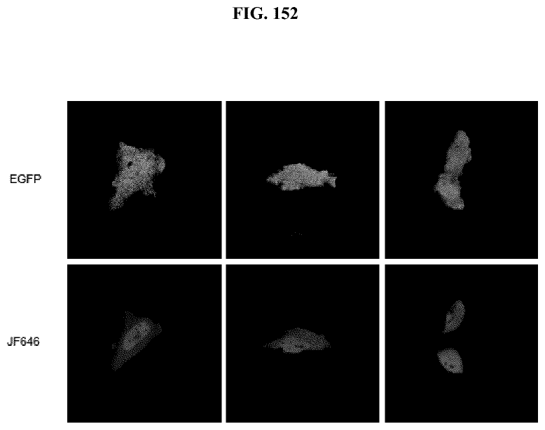

The same dual tag on BRD4 at endogenous levels can be used to detect other interactions, such as the interaction with VHL E3 ligase after ternary complex formation by addition of the MZ1 PROTAC ligand (FIG. 152).

### Example 13

### Improved Performance of Large HaloTag (HaloTag[22-297](M2F)) Mutants in Mammalian Cell Assays

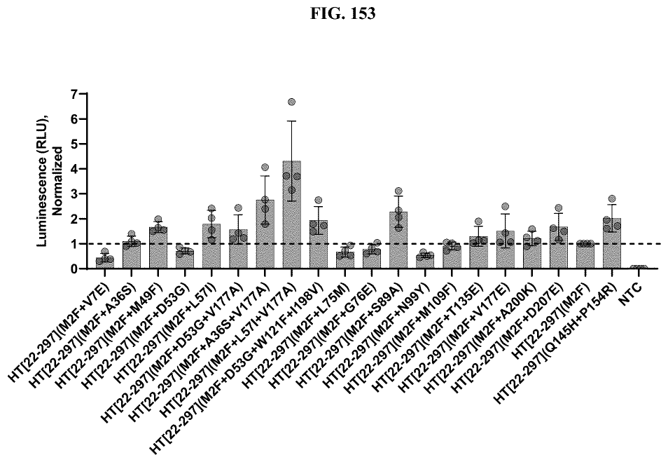

Experiments were conducted during development of embodiments herein to demonstrate that improvement in the expression of LgHT in mammalian cells can be done by introducing mutations, measured by a HiBiT lytic assay with the tagged mutants (FIG. 153). Preferred variants from this testing were M49F, L57I, S89A, and the double/triple mutants shown.

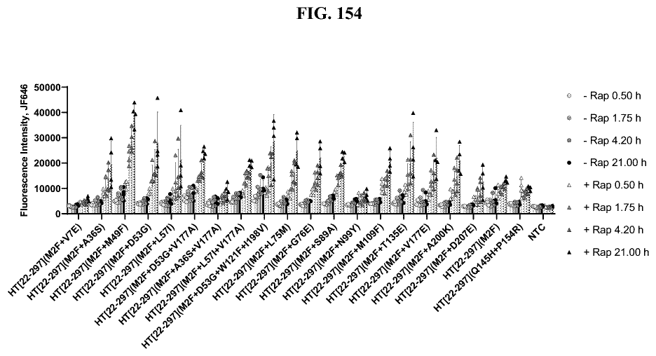

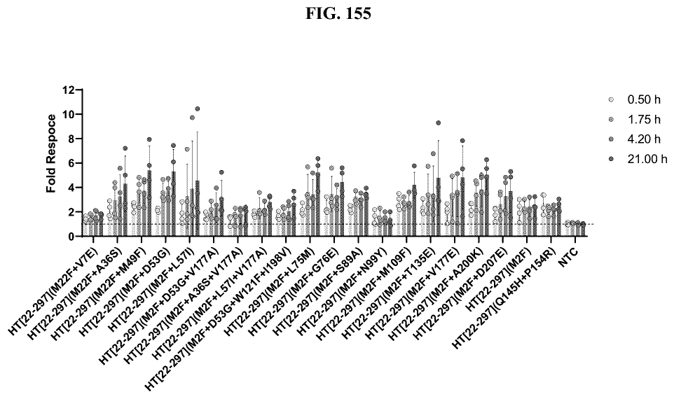

In addition to improving expression, mutations in the LgHT can also improve performance in protein:protein interaction assays in live mammalian cells by fusing the mutant LgHT to FRB (FIGS. 154-155). In particular, the notable mutants were M49F, T135E, S89A, V177E, D53G, and L57I, in addition to the double/triple mutants shown.

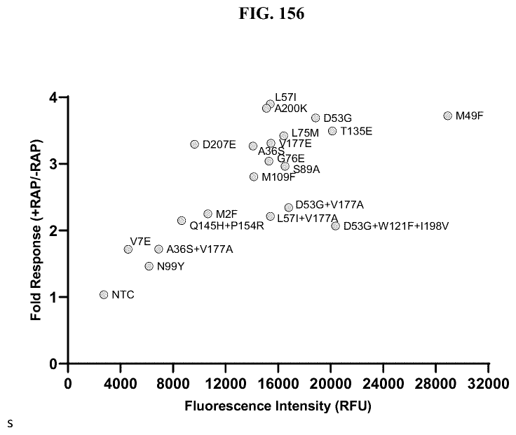

Mutants were identified that improved both the fold response and maximum fluorescence of the LgHT (FIG. 156).

1-2. (canceled)

3. A composition comprising a split variant of a polypeptide comprising (i) a first fragment of the polypeptide comprising at least 70% sequence identity with a first portion of SEQ ID NO: 1, and (ii) a second fragment of the polypeptide comprising at least 70% sequence identity with a second portion of SEQ ID NO: 1.

4-8. (canceled)

9. The composition of claim 3, wherein:
the first fragment comprises at least 70% sequence identity with a first reference sequence selected from one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, and 576; and
the second fragment comprises at least 70% sequence similarity with a second reference sequence selected from one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, and 577.

10-11. (canceled)

12. The composition of claim 3, wherein the split variant comprises a sp site at a position corresponding to a position between positions 5 and 13, 36 and 51, 63 and 72, 84 and 92, 104 and 130, 142 and 148, 160 and 174, 186 and 189, 311 and 313, 221 and 229, or 269 and 290, of SEQ ID NO: 1.

13. The composition of claim 12, wherein the split variant comprises deletions of up to 40 amino acids at positions corresponding to one or more of the N-terminus of SEQ ID NO: 1, the C-terminus of SEQ ID NO: 1, and either side of the sp site.

14. The composition of claim 12, wherein the split variant comprises duplicated sequences of up to 40 amino acids at positions corresponding to either side of the sp site.

15. The composition of claim 3, wherein the split variant is capable of forming a covalent bond with a haloalkane substrate.

16. The composition of claim 3, wherein the first fragment is present as a fusion protein with a first peptide, polypeptide, or protein of interest.

17. The composition of claim 16, wherein the first peptide, polypeptide, or protein of interest is selected from the group consisting of an antibody, antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, oligonucleotide probe, peptide nucleic acid, DARPin, anticalin, nanobody, aptamer, affimer, a purified protein, and analyte binding domain(s) of proteins.

18. The composition of claim 16, wherein the second fragment is present as a fusion protein with a second peptide, polypeptide, or protein of interest.

19. The composition of claim 18, wherein the second peptide, polypeptide, or protein of interest is selected from the group consisting of an antibody, antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, oligonucleotide probe, peptide nucleic acid, DARPin, anticalin, nanobody, aptamer, affimer, a purified protein, and analyte binding domain(s) of proteins.

20. The composition of claim 18, wherein the first and second peptides, polypeptides, or proteins of interest are interaction elements capable of forming a complex with each other.

21. The composition of claim 20, wherein the first fragment and the second fragment are incapable of associating with each other under physiological conditions in the absence of the association of the first and second interaction elements.

22. The composition of claim 16, wherein the first and second peptides, polypeptides, or proteins of interest are co-localization elements configured to co-localize within a cellular compartment, a cell, a tissue, or an organism.

23. The composition of claim 16, wherein the second fragment is tethered to a molecule of interest.

24-31. (canceled)

32. A polynucleotide or polynucleotides encoding the composition of claim 1.

33. An expression vector or expression vectors comprising the polynucleotide or polynucleotides of claim 32.

34. A host cell comprising the polynucleotide of polynucleotide or polynucleotides of claim 32.

35-43. (canceled)

44. A method comprising to detect a protein-protein interaction in a sample comprising contacting:
(a) a first fusion comprising:
(i) a first complementary fragment of a split variant of a polypeptide comprising at least 70% sequence similarity with a first portion of SEQ ID NO: 1; and
(ii) a first protein of interest;
(b) a second fusion comprising:
(i) a second complementary fragment of a split variant of a polypeptide comprising at least 70% sequence similarity with a second portion of SEQ ID NO: 1; and
(ii) a second protein of interest; and
(c) a substrate comprising R-linker-A-X, wherein R is a functional group or solid support, X is a halogen, and A-X is a substrate for a dehalogenase enzyme;
wherein binding of the first protein of interest to the second protein of interest results in formation of a complex between the first complementary fragment and the secondary complementary fragment that is capable for forming a covalent bond with the substrate.

45-46. (canceled)
